(19) United States
(12) Patent Application Publication
(10) Pub. No.: US 2016/0138077 A1
(43) Pub. Date: May 19, 2016

(54) MONITORING AND ASSESSING DEACETYLASE ENZYME ACTIVITY

(71) Applicant: THE GENERAL HOSPITAL CORPORATION, Boston, MA (US)

(72) Inventors: Jacob HOOKER, Belmont, MA (US); Himashinie DIYABALANAGE, Charlestown, MA (US)

(73) Assignee: THE GENERAL HOSPITAL CORPORATION, Boston, MA (US)

(21) Appl. No.: 14/899,882

(22) PCT Filed: Jun. 17, 2014
(86) PCT No.: PCT/US2014/042667

§ 371 Date: Dec. 18, 2015

Related U.S. Application Data
(60) Provisional application No. 61/837,213, filed on Jun. 20, 2013.

(51) Int. Cl. C12Q 1/34; A61K 47/48; C07D 271/12

(52) U.S. Cl. CPC C12Q 1/34; C07D 271/12; A61K 47/48061; G01N 2333/98; G01N 2500/04; G01N 2500/10

(57) ABSTRACT

Disclosed herein are compounds and methods for detecting enzyme activity. In some embodiments, the enzyme is a deacetylase enzyme such as HDAC. The compound of the invention comprises a detectable label, a linker, and an enamide group. The compound can be enzymatically cleaved by the enzyme of interest to produce a nucleophilic fragment that includes the detectable label. Measurement of a signal generated by the detectable label can indicate enzyme activity. The compounds can be used as either in vivo or in vitro enzyme probes.

### CROSS-REFERENCE TO RELATED APPLICATION

This application claims benefit under 35 U.S.C. §119(e) of U.S. Provisional Application No. 61/837,213 filed Jun. 20, 2013, the contents of which are incorporated herein by reference in its entirety.

### GOVERNMENT SUPPORT

This invention was made with government support under grant 5R01DA030321 awarded by the National Institutes of Health (NIH). The government has certain rights in the invention.

### TECHNICAL FIELD

The present disclosure relates to enzyme activity detection, cell targeting, and drug delivery.

### BACKGROUND

It is of great importance to be able to detect or measure enzyme activity within the context of natural cellular environment. For example, this is particularly relevant for drug development, in which in vivo enzyme activity measurement can facilitate the functional validation of pharmaceutical targets.

Enzymatic modification of small molecule imaging probes is used to detect changes in enzyme expression or activation within cells, tissues, or organisms (Johnsson & Johnsson, ACS Chem. Biol. 2007, 2, 31-38; Prescher & Bertozzi, Nat. Chem. Biol. 2005, 1, 13-21; Kobayashi et al., Chem. Rev. 2010, 110, 2620-2640; Baruch et al, Trends Cell Biol. 2004, 14, 29-35; Blum et al. Nat. Chem. Biol. 2005,1, 203-209). Although activity-based enzyme probes are widely used for in vitro and cellular studies, translation to in vivo imaging studies can be limited when the probe design lacks a method for cellular or tissue retention following interaction with an enzyme target (Blum et al. Nat. Chem. Biol. 2005, 1, 203-209; Weissleder et al., Nat. Biotech. 1999, 17, 375-378; Wysocki & Lavis, Curr. Opin. Chem. Biol. 2011, 15, 752-759; Tian et al., P. Natl. Acad. Sci. USA. 2012, 109, 4756-4761; Yeh et al., NeuroImage 2012, 64, 630-639; Cheng et al. J. Am. Chem. Soc. 2012, 134, 3103-3110; Baba et al., J. Am. Chem. Soc. 2012, 134, 14310-14313; Sasaki et al., Bioorga. Med. Chem. 2012, 20, 1887-1892).

Accordingly, there is a need in the art for novel probes that can detect or measure enzyme activity in vivo.

### SUMMARY

The invention provides, inter alia, a compound for detecting or measuring deacetylase enzyme activity in vitro or in vivo, the compound characterized in having a structure: Lab-L-Ena, where Lab is a detectable label, L is a linker, and Ena is an enamide group.

The inventors have discovered that a deacetylase enzyme such as histone deacetylase can cleave the compound described herein to generate a nucleophilic fragment which can be localized within a cell. Because the nucleophilic fragment comprises the detectable label, a signal produced by the detectable label can be used to indicate or quantify enzyme activity.

In some embodiments, the compound corresponds to Formula I:

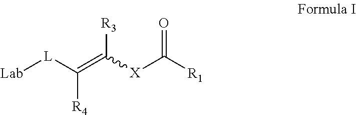

where X is O, S, or NR₂; and R₁, R₂, R₃, and R₄ are each independently hydrogen, deuterium, halogen, hydroxyl, nitro, cyano, isocyano, thiocyano, isothiocyano, aryl, alkyl, perfluorinated alkyl, alkenyl, perfluorinated alkenyl, alkynyl, perfluorinated alkynyl, alkoxy, alkylthioxy, amino, monoalkylamino, dialkylamino, acyl, carbonyl, carboxyl, azide, sulfinyl, sulfonyl, sulfino, sulfo, or thiol, each of which can be optionally substituted and each of which can optionally comprise a stable isotope.

In some embodiments, the detectable label is an imagining agent or a contrast agent.

In some embodiments, the detectable label is selected from a group consisting of an optical reporter, non-metallic isotope, a paramagnetic metal ion, a ferromagnetic metal, echogenic substance (either liquid or gas), a boron neutron absorber, a gamma-emitting radioisotope, a positron-emitting radioisotope, and an x-ray absorber.

In some embodiments, the detectable label is selected from a group consisting of fluorescent molecules, radioisotopes, nucleotide chromophores, enzymes, enzyme substrates, chemiluminescent moieties, magnetic particles, bioluminescent moieties, nucleic acids, antibodies, and any combinations thereof.

In some embodiments, the compound corresponds to Formula II:

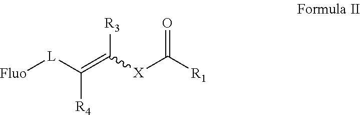

where X is O, S, or NR₂; and R₁, R₂, R₃, and R₄ are each independently hydrogen, deuterium, halogen, hydroxyl, nitro, cyano, isocyano, thiocyano, isothiocyano, aryl, alkyl, perfluorinated alkyl, alkenyl, perfluorinated alkenyl, alkynyl, perfluorinated alkynyl, alkoxy, alkylthioxy, amino, monoalkylamino, dialkylamino, acyl, carbonyl, carboxyl, azide, sulfinyl, sulfonyl, sulfino, sulfo, or thiol, each of which can be optionally substituted and each of which can optionally comprise a stable isotope.

In some embodiments, the fluorescent molecule comprises hydroxycoumarin, aminocoumarin, methoxycoumarin, cascade blue, pacific blue, pacific orange, lucifer yellow, nitrobenzoxadiazole (NBD), R-phycoerythrin, PE-Cy5 conjugates, PE-Cy7 conjugates, Red 613, PerCP, TruRed, FluorX, Fluorescein, BODIPY, Cyt, Cy3, Cy3B, Cy3.5, Cy5, Cy5.5, Cy7, SeTau-647, TRITC, rhodamine, Texas Red, allophycocyanin (APC), APC-Cy7 conjugates, or derivatives thereof.

In some embodiments, the fluorescent molecule comprises NBD, and the compound corresponds to Formula III:

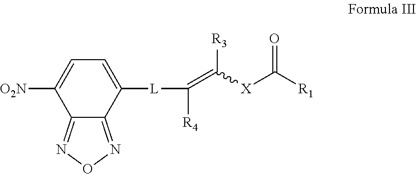

where X is O, S, or NR₂; and R₁, R₂, R₃, and R₄ are each independently hydrogen, deuterium, halogen, hydroxyl, nitro, cyano, isocyano, thiocyano, isothiocyano, aryl, alkyl, perfluorinated alkyl, alkenyl, perfluorinated alkenyl, alkynyl, perfluorinated alkynyl, alkoxy, alkylthioxy, amino, monoalkylamino, dialkylamino, acyl, carbonyl, carboxyl, azide, sulfinyl, sulfonyl, sulfino, sulfo, or thiol, each of which can be optionally substituted and each of which can optionally comprise a stable isotope.

In some embodiments, the linker is selected from the group consisting of: —O—, —S—, —S—S—, —NR^{a}—, —C(O)—, —C(O)O—, —C(O)NR^{a}—, —SO—, —SO₂—, —SO₂NR^{a}—, substituted or unsubstituted alkyl, substituted or unsubstituted alkenyl, substituted or unsubstituted alkynyl, arylalkyl, arylalkenyl, arylalkynyl, heteroarylalkyl, heteroarylalkenyl, heteroarylalkynyl, heterocyclylalkyl, heterocyclylalkenyl, heterocyclylalkynyl, aryl, heteroaryl, heterocyclyl, cycloalkyl, cycloalkenyl, alkylarylalkyl, alkylarylalkenyl, alkylarylalkynyl, alkenylarylalkyl, alkenylarylalkenyl, alkenylarylalkynyl, alkynylarylalkyl, alkynylarylalkenyl, alkynylarylalkynyl, alkylheteroarylalkyl, alkylheteroarylalkenyl, alkylheteroarylalkynyl, alkenylheteroarylalkyl, alkenylheteroarylalkenyl, alkenylheteroarylalkynyl, alkynylheteroarylalkyl, alkynylheteroarylalkenyl, alkynylheteroarylalkynyl, alkylheterocyclylalkyl, alkylheterocyclylalkenyl, alkylhererocyclylalkynyl, alkenylheterocyclylalkyl, alkenylheterocyclylalkenyl, alkenylheterocyclylalkynyl, alkynylheterocyclylalkyl, alkynylheterocyclylalkenyl, alkynylheterocyclylalkynyl, alkylaryl, alkenylaryl, alkynylaryl, alkylheteroaryl, alkenylheteroaryl, alkynylhereroaryl; wherein backbone of the linker can be interrupted or terminated by O, S, S(O), SO₂, N(R^{a})₂, C(O), C(O)O, C(O)NR^{a}, cleavable linking group, substituted or unsubstituted aryl, substituted or unsubstituted heteroaryl, substituted or unsubstituted heterocyclic, and wherein R^{a} is hydrogen, acyl, aliphatic or substituted aliphatic.

In some embodiments, the compound corresponds to Formula IV:

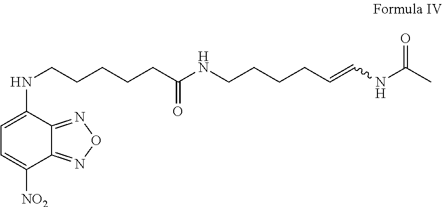 [SMILES: CC(=O)NC=CCCCCNC(=O)CCCCCNc1ccc([N+](=O)[O-])c2nonc12]

In some embodiments, the compound is a trans-isomer.

In another aspect, the invention provides a method of detecting enzyme activity of a deacetylase enzyme, the method comprising contacting the deacetylase enzyme with a compound described herein, and determining the deacetylase activity by measuring a signal produced by a fragment of the compound.

In some embodiments, the deacetylase enzyme is a histone deacetylase (HDAC) or a sirtuin.

In some embodiments, the deacetylase enzyme is one of Class I HDAC enzymes.

In some embodiments, the deacetylase enzyme is HDAC1, HDAC3, or a combination thereof.

In some embodiments, the signal is a fluorescent signal, a magnetic signal, or a radioactive signal.

In some embodiments, the fragment of the compound is produced by the deacetylase enzyme cleaving the compound.

In some embodiments, the contacting is ex vivo.

In some embodiments, the contacting is in vivo.

In some embodiments, the deacetylase enzyme is within a cell, and wherein the signal is localized within the cell.

In some embodiments, the method further comprises administering the compound to a subject comprising the cell.

In some embodiments of in vivo contacting, the compound is administered in a pharmaceutically-acceptable carrier.

In some embodiments, the subject is a mammal.

In some embodiments, the mammal is a human.

In another related aspect, the invention provides a method of screening a substance for its effect on deacetylase enzyme activity, the method comprising contacting the substance with a deacetylase enzyme, contacting the deacetylase enzyme with a compound described herein, and determining the effect of the substance on deacetylase enzyme activity by measuring and comparing a signal produced by a fragment of the compound relative to a control, wherein the control is performed in the absence of the substance.

In some embodiments, the deacetylase enzyme is a histone deacetylase (HDAC) or a sirtuin.

In some embodiments, the deacetylase enzyme is one of Class I HDAC enzymes.

In some embodiments, the deacetylase enzyme is HDAC1, HDAC3, or a combination thereof.

In some embodiments, the signal is a fluorescent signal, a magnetic signal, or a radioactive signal.

In some embodiments, the fragment of the compound is produced by the deacetylase enzyme cleaving the compound.

In some embodiments, the substance enhances deacetylase enzyme activity if the signal is above a reference level determined from the control.

In some embodiments, the substance reduces deacetylase enzyme activity if the signal is below a reference level determined from the control.

In some embodiments, the deacetylase enzyme is within a cell.

Another aspect of the invention relates to the use of the compound described herein to detect deacetylase enzyme activity.

In yet another aspect, a method is provided herein for targeting a cell comprising a deacetylase enzyme within a cell population, the method comprising contacting the cell population with the compound described herein

In some embodiments, the deacetylase enzyme is a histone deacetylase (HDAC) or a sirtuin.

In some embodiments, the deacetylase enzyme is one of Class I HDAC enzymes.

In some embodiments, the deacetylase enzyme is HDAC1, HDAC3, or a combination thereof.

In a further aspect, a method is provided herein for delivering a drug to a cell comprising a deacetylase enzyme, the method comprising contacting the cell with a composition comprising the drug linked to an enamide group.

In some embodiments, the deacetylase enzyme is a histone deacetylase (HDAC) or a sirtuin.

In some embodiments, the deacetylase enzyme is one of Class I HDAC enzymes.

In some embodiments, the deacetylase enzyme is HDAC1, HDAC3, or a combination thereof.

Yet another aspect of the invention relates to a method of forming a nucleophile, the method comprising contacting a deacetylase enzyme with the compound described herein, whereby the deacetylase enzyme cleaves the compound to form the nucleophile.

### BRIEF DESCRIPTION OF THE DRAWINGS

FIG. 1 is a schematic showing cell-localized, activity-based enzyme detection. Cleavage of the amide bond of the enamide followed by conversion to aldehyde leads to increased cellular retention due to reaction with adventitious intracellular nucleophiles.

FIG. 2 is a schematic showing that the synthesis of HP-1 is achieved in 9 steps and enzymatic deacetylation of HP-1 forms DHP-1.

FIGS. 3A-3B show synthesis procedures for HP-1.

FIG. 3A. Building block synthesis for HP-1.

FIG. 3B. Attachment to the NBD fluorophore. Reagents and conditions (i) AIBN, Bu₃SnH, THF, 90° C., 12 hours, 90% (ii) I₂, CH₂Cl₂, 30 min, room temperature (RT), 61% (iii) t-Bu(Cl)Ph₂Si, Im, CH₂Cl₂, overnight, 0° C., 73% (iv) CuI, CH₃CONH₂, Cs₂CO₃, DMEDA, THF, 20 h, 65° C., 72% (v) TBAF, THF, overnight, RT, 80% (vi) Pyridine, TsCl, DCM, overnight, RT, 64% (vii) NaN₃, DMF, 3 h, 80° C., 81% (viii) NH₄Cl, Zn, EtOH, H₂O, 3 h, RT, 80% (ix) Et₃N, CH₂Cl₂, RT, overnight, 75%.

FIG. 4 shows synthesis procedures for HP-2. Reagents and conditions: (x) CH₂Cl₂, RT, 3 hours, 65% (xi) 1M NaOH, acetic anhydride, RT, overnight, 80%.

FIGS. 5A-5B show data evaluating the stability of the enamide (compound 9).

FIG. 5A. HPLC analysis of acid hydrolysis of 9 over time at pH 2.

FIG. 5B. HPLC analysis of 9 after 60 min at pH 4, 6, 7, 8, 10 and 12.

FIGS. 6A-6D show LCMS characterization of HDAC enzymatic action on HP-1 and HP-2.

FIG. 6A. LCMS analysis of 1.5:1 trans and cis isomer mixture of HP-1.

FIG. 6B. LCMS analysis of HP-1 isomer mixture deacetylation by HDAC3 enzyme.

FIG. 6C. Graph of the production of DHP-1 following HP-1 deacetylation by HDAC3 over 12 hours.

FIG. 6D. Graph of the natural log of DHP-1 formation versus time for calculation of the observed rate constant and the life time (T_{1/2}) of the enzyme-catalyzed reaction.

FIGS. 7A-7F are representative IC₅₀ curves for HP-1 and HDAC enzymes using Trypsin-coupled assay and Caliper assay.

FIG. 7A. HP-1 IC₅₀ curve for HDAC1 by Trypsin-Coupled Assay.

FIG. 7B. HP-1 IC₅₀ curve for HDAC1 by Caliper Assay.

FIG. 7C. HP-1 IC₅₀ curve for HDAC2 by Caliper Assay.

FIG. 7D. HP-1 IC₅₀ curve for HDAC3 by Caliper Assay.

FIG. 7E. HP-1 IC₅₀ curve for HDAC6 by Caliper Assay.

FIG. 7F. HP-1 IC₅₀ curve for HDAC8 by Caliper Assay.

FIGS. 8A-8F are representative IC₅₀ curves for HP-2 and HDAC enzymes using Trypsin-coupled assay and Caliper assay.

FIG. 8A. HP-2 IC₅₀ curve for HDAC1 by Trypsin-Coupled Assay.

FIG. 8B. HP-2 IC₅₀ curve for HDAC1 by Caliper Assay.

FIG. 8C. HP-2 IC₅₀ curve for HDAC2 by Caliper Assay.

FIG. 8D. HP-2 IC₅₀ curve for HDAC3 by Caliper Assay.

FIG. 8E. HP-2 IC₅₀ curve for HDAC6 by Caliper Assay.

FIG. 8F. HP-2 IC₅₀ curve for HDAC8 by Caliper Assay.

FIGS. 9A-9B show that the unmasked aldehyde DHP-1 is produced by enzymatic deacetylation and forms conjugates with adventitious nucleophiles on proteins.

FIG. 9A. Mechanism of increased intracellular retention of HP-1 following conversion to DHP-1.

FIG. 9B. Ratio of fluorescence from the protein-DHP-1 conjugate (fraction 2) and unbound HP-1 and DHP-1 (fraction 6) collected during gel filtration chromatography of reactions A-H (A: HP-1; B: HP-1 and NaCNBH₃; C: HP-1 and BSA; D: HP-1, NaCNBH₃, and BSA; E: HP-1 and HDAC3; F: HP-1, HDAC3, and NaCNBH₃; G: HP-1, HDAC3, and BSA; H: HP-1, HDAC3, NaCNBH₃, and BSA). In the presence of BSA, DHP-1-protein conjugation occurs in the absence (i, p<0.001) or presence (ii, p<0.001) of NaCNBH₃. In the absence of BSA, HDAC3 deacetylates HP-1 and conjugates with DHP-1 in the presence of NaCNBH₃ (iii, p<0.001). Statistical analyses were performed with a two-tailed Student's t-test. A-H, n=3 and error bars indicate ±SD.

FIG. 10 are representative images of fluorescence from fractions collected following gel filtration chromatography of reactions A-H (A: HP-1; B: HP-1 and NaCNBH3; C: HP-1 and BSA; D: HP-1, NaCNBH₃, and BSA; E: HP-1 and HDAC3; F: HP-1, HDAC3, and NaCNBH3; G: HP-1, HDAC3, and BSA; H: HP-1, HDAC3, NaCNBH3, and BSA). Columns with fractions 2 and 6, which were used to calculate the F2/F6 ratio for FIG. 9B, are outlined in the boxes.

FIGS. 11A-11J show that cellular accumulation of enamide probe HP-1 is sensitive to HDAC activity.

FIG. 11A. Trapping of HP-1 in HeLa cell lysate.

FIGS. 11B-11E. Confocal microscopy images of HeLa cells in the absence (FIGS. 11B-11C) or presence (FIGS. 11D-11E) of 10 μM SAHA, added 15 min prior to incubation with 5 μM HP-1 for 2 hours. Scale bars=20 μm.

FIGS. 11B and 11D. Intracellular NBD fluorescence.

FIGS. 11C and 11E. DAPI nuclear stain with brightfield overlay.

FIGS. 11F-11I. Confocal microscopy images of HeLa cells in the absence (FIGS. 11F-11G) or presence (FIGS. 11H-11I) of 10 μM SAHA, added 15 min prior to incubation with 5 μM HP-2 for 2 hours. Scale bars=20 μm.

FIGS. 11F and 11H. Intracellular NBD fluorescence.

FIGS. 11G and 11I. DAPI nuclear stain with brightfield overlay.

FIG. 11J. Mean NBD fluorescence intensity of cells with 5 μM HP-1 or HP-2±10 μM SAHA, n=9, error bars indicate ±SD.

### DETAILED DESCRIPTION

The invention is based, inter alia, on designing an enzyme probe that can be cleaved by an enzyme of interest in vitro or in vivo to form a trappable nucleophilic fragment for cellular localization. The enzyme probe and the trappable nucleophilic fragment thereof comprises a signal portion that is capable of producing a measurable signal such as an optical signal, a magnetic signal, an electronic signal, or a radioactive signal. Thus detection of such a signal can be used to indicate and quantify enzyme activity. In addition to providing a mechanism for intracellular trapping, this enzyme probe strategy transcends the limitations of many in vitro imaging strategies, as its modular design makes it suitable for labeling with a variety of detectable lables (e.g., an imaging agent, a contrast agent, or a radioisotope).

Guided by this design principle, the inventors have discovered that by conjugating an enamide group with a fluorescent molecule (FIG. 1), the resultant composition functions surprisingly well at detecting deacetylase activity, even in vivo. More specifically, the enamide group permits a deacetylase enzyme to cleave the composition in an efficient manner and form an aldehyde fragment conjugated to the fluorescent molecule. The aldehyde-fluorescent molecule conjugate can subsequently localize within the cell, for example, in the cytoplasm. Finally, the fluorescent molecule can emit fluorescence to report the deacetylase activity. In comparison, a control compound without the enamide group is enzymatically cleaved at significanitly lower efficiency and does not result in localization within the cell. Thus, the compositions and methods described herein aim to exploit this discovery and the design principle, and to provide useful compositions and methods for enzyme activity detection either in vitro or in vivo. As used herein, the term “enamide group” refers to a chemical group having the following structure:

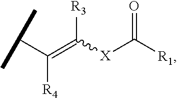

where the bold line denotes the point of attachment to another molecular fragment, the squiggly line indicates that the enamide group can exist in either a cis or trans configuration, X is O, S, or NR₂, and R₁, R₂, R₃, and R₄ are each independently hydrogen, deuterium, halogen, hydroxyl, nitro, cyano, isocyano, thiocyano, isothiocyano, aryl, alkyl, perfluorinated alkyl, alkenyl, perfluorinated alkenyl, alkynyl, perfluorinated alkynyl, alkoxy, alkylthioxy, amino, monoalkylamino, dialkylamino, acyl, carbonyl, carboxyl, azide, sulfinyl, sulfonyl, sulfino, sulfo, or thiol, each of which can be optionally substituted and each of which can optionally comprise a stable isotope.

One aspect of the invention relates to a compound characterized in having a structure: Lab-L-Ena, wherein Lab is a detectable label, L is a linker, and Ena is an enamide group. As used herein, “detectable label” refers to an element or functional group capable of producing a detectable signal indicative of the presence of a target, e.g., element or functional group detectable by spectroscopic, photochemical, biochemical, immunochemical, electrical, optical, radiation or chemical means. An optical signal can include, but is not limited to, fluorescence, a visible color, an infrared signal, or an ultraviolet signal.

The compound described herein can be used for detecting or measuring deacetylase enzyme activity. There are at least two families of deacetylase enzymes identified in eukaryotes, the histone deacetylases (HDACs), and the Sir2-like deacetylases or sirtuins.

Histones are proteins found in eukaryotic cell nuclei and are involved in the packaging and ordering of DNA into structural units called nucleosomes. They are the chief protein components of chromatin acting as spools around which DNA winds. Histone tails are normally positively charged due to the protonation of amine groups on lysine and arginine amino acids. These positive charges help the histone tails to interact with and bind to the negatively charged phosphate groups on the DNA backbone. HDACs remove the acetyl groups from the lysine residues on the histones, increasing the positive charge of histone tails and encouraging binding between histones and the DNA backbone. The increased DNA binding condenses DNA structure and prevents transcription. In addition to the role HDACs play in modifying histones, they have also been found to deacetylate a broad array of other proteins (Science 2009, 325, 834-840).

HDACs are classified into four classes: Class I that includes HDAC1, HDAC2, HDAC3, and HDAC8; Class IIA that includes HDAC4, HDAC5, HDAC7, and HDAC9; Class IIB that includes HDAC6 and HDAC10; Class III that includes sirtuins in mammals (e.g., SIRT1, SIRT2, SIRT3, SIRT4, SIRT5, SIRT6, or SIRT7) and Sir2 in the yeast S. cerevisiae; and Class IV that includes, HDAC11. More information about the deacetylase enzymes can be found in “Deacetylase Enzymes: Biological Functions and the Use of Small-Molecule Inhibitors” by Grozinger and Schreiber (Chemistry & Biology 2002, 9, 3-16), and “Histone deacetylase inhibitors in cancer therapy” by Land and Chabner (J Clin Oncol. 2009, 27, 5459-68), the contents of each of which are incorporated by reference in its entirety.

HDACs have been recognized as potentially useful therapeutic targets for a broad range of human disorders including cancer and diabetes. Thus, assays for measuring HDAC enzyme activity are particularly useful in basic research and aiding the development of new HDAC inhibitors.

Sirtuins are classified into five classes: Class I that includes SIRT1, SIRT2, and SIRT3; Class II that includes SIRT4; Class III that includes SIRT5; Class IV that includes SIRT6 and SIRT7; and Class V that includes sirtuins that are intermediate in sequence between classes.

In some embodiments, the deacetylase enzyme is one of Class I HDAC enzymes. In some embodiments, the deacetylase enzyme is HDAC1, HDAC3, or a combination thereof.

In some embodiments, the compound corresponds to Formula I:

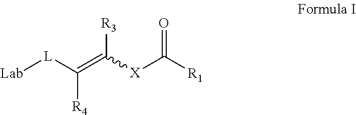

In compounds of Formula I, X can be O, S, or NR₂.

In compounds of Formula I, R₁, R₃, and R₄ are each independently hydrogen, deuterium, halogen, hydroxyl, nitro, cyano, isocyano, thiocyano, isothiocyano, aryl, alkyl, perfluorinated alkyl, alkenyl, perfluorinated alkenyl, alkynyl, perfluorinated alkynyl, alkoxy, alkylthioxy, amino, monoalkylamino, dialkylamino, acyl, carbonyl, carboxyl, azide, sulfinyl, sulfonyl, sulfino, sulfo, or thiol, each of which can be optionally substituted and each of which can optionally comprise a stable isotope.

In compounds of Formula I, at least one (e.g., one, two, or three) of R₁, R₃, and R₄ can be hydrogen. In some embodiments, R₁ is hydrogen. In some embodiments, R₃ is hydrogen. In some embodiments, R₄ is hydrogen.

In compounds of Formula I, at least one (e.g., one, two, or three) of R₁, R₃, and R₄ can be deuterium. In some embodiments, R₁ is deuterium. In some embodiments, R₃ is deuterium. In some embodiments, R₄ is deuterium.

In compounds of Formula I, at least one (e.g., one, two, or three) of R₁, R₃, and R₄, can be optionally substituted C₁-C₆ alkyl. In some embodiments, R₁ is optionally substituted C₁-C₆ alkyl. In some embodiments, R₃ is optionally substituted C₁-C₆ alkyl. In some embodiments, R₄ is optionally substituted C₁-C₆ alkyl. Exemplary C₁-C₆ alkyls include, but are not limited to, methyl, ethyl, propyl, allyl, propargyl, butyl, but-2-yl, 2-methylpropyl, and pentyl. In some embodiments, at least one (e.g., one, two, or three) of R₁, R₃, and R₄ is a methyl. In some embodiments, the optionally substituted C₁-C₆ alkyl is perfluorinated C₁-C₆ alkyl. Exemplary perfluorinated C₁-C₆ alkyls include, but are not limited to, —CF₃, —C₂F₅, —C₃F₇, —C₄F₉, —C₅F₁₁, and —C₆F₁₃.

In compounds of Formula I, at least one (e.g., one, two, or three) of R₁, R₃, and R₄, can be optionally substituted C₂-C₆ alkenyl. In some embodiments, R₁ is optionally substituted C₂-C₆ alkenyl. In some embodiments, R₃ is optionally substituted C₂-C₆ alkenyl. In some embodiments, R₄ is optionally substituted C₂-C₆ alkenyl. Exemplary C₂-C₆ alkenyls include, but are not limited to, ethenyl, 2-propenyl, 2-butenyl, 3-butenyl, 2-methyl-2-propenyl, 3-methyl-2-butenyl, 2-pentenyl, and 2-hexenyl. In some embodiments, the optionally substituted C₂-C₆ alkenyl is perfluorinated C₂-C₆ alkenyl. Exemplary perfluorinated C₂-C₆ alkenyls include, but are not limited to, —CF═CF₂, —CF₂—CF═CF₂, CF₂—CF₂—CF═CF₂.

In compounds of Formula I, at least one (e.g., one, two, or three) of R₁, R₃, and R₄, can be optionally substituted C₂-C₆ alkynyl. In some embodiments, R₁ is optionally substituted C₂-C₆ alkynyl. In some embodiments, R₃ is optionally substituted C₂-C₆ alkynyl. In some embodiments, R₄ is optionally substituted C₂-C₆ alkynyl. Exemplary C₂-C₆ alkynyls include, but are not limited to, ethynyl, prop-1-yn-1-yl, prop-2-yn-1-yl, n-but-1-yn-1-yl, n-but-1-yn-3-yl, n-but-1-yn-4-yl, n-but-2-yn-1-yl, n-pent-1-yn-1-yl, n-pent-1-yn-3-yl, n-pent-1-yn-4-yl, n-pent-1-yn-5-yl, n-pent-2-yn-1-yl, n-pent-2-yn-4-yl, n-pent-2-yn-5-yl, 3-methylbut-1-yn-3-yl, 3-methylbut-1-yn-4-yl, n-hex-1-yn-1-yl, n-hex-1-yn-3-yl, n-hex-1-yn-4-yl, n-hex-1-yn-5-yl, n-hex-1-yn-6-yl, n-hex-2-yn-1-yl, n-hex-2-yn-4-yl, n-hex-2-yn-5-yl, n-hex-2-yn-6-yl, n-hex-3-yn-1-yl, n-hex-3-yn-2-yl, 3-methylpent-1-yn-1-yl, 3-methylpent-1-yn-3-yl, 3-methylpent-1-yn-4-yl, 3-methylpent-1-yn-5-yl, 4-methylpent-1-yn-1-yl, 4-methylpent-2-yn-4-yl or 4-methylpent-2-yn-5-yl. In some embodiments, the optionally substituted C₂-C₆ alkynyl is perfluorinated C₂-C₆ alkynyl. Exemplary perfluorinated C₂-C₆ alkynyls include, but are not limited to, —C≡CF, —CF₂-C≡CF, —CF₂—CF₂-C≡CF.

In compounds of Formula I, at least one (e.g., one, two, or three) of R₁, R₃, and R₄, can be an optionally substituted aryl or heteroaryl. In some embodiments, R₁ is an optionally substituted aryl or heteroaryl. In some embodiments, R₃ is an optionally substituted aryl or heteroaryl. In some embodiments, R₄ is an optionally substituted aryl or heteroaryl. In some embodiments, the aryl is phenyl.

In compounds of Formula I, at least one (e.g., one, two, or three) of R₁, R₃, and R₄, can be halogen. In some embodiments, R₁ is halogen. In some embodiments, R₃ is halogen. In some embodiments, R₄ is halogen.

In compounds of Formula I, at least one (e.g., one, two, or three) of R₁, R₃, and R₄, can be optionally substituted alkoxy. In some embodiments, R₁ is optionally substituted alkoxy. In some embodiments, R₃ is optionally substituted alkoxy. In some embodiments, R₄ is optionally substituted alkoxy.

In compounds of Formula I, at least one (e.g., one, two, or three) of R₁, R₃, and R₄, can be nitro. In some embodiments, R₁ is nitro. In some embodiments, R₃ is nitro. In some embodiments, R₄ is nitro.

In compounds of Formula I, at least one (e.g., one, two, or three) of R₁, R₃, and R₄, can be cyano. In some embodiments, R₁ is cyano. In some embodiments, R₃ is cyano. In some embodiments, R₄ is cyano.

When X is NR₂, R₂ can be independently hydrogen, deuterium, halogen, hydroxyl, nitro, cyano, isocyano, thiocyano, isothiocyano, aryl, alkyl, perfluorinated alkyl, alkenyl, perfluorinated alkenyl, alkynyl, perfluorinated alkynyl, alkoxy, alkylthioxy, amino, monoalkylamino, dialkylamino, acyl, carbonyl, carboxyl, azide, sulfinyl, sulfonyl, sulfino, sulfo, or thiol, each of which can be optionally substituted and each of which can optionally comprise a stable isotope. In some embodiments, R₂ is hydrogen. In some embodiments, R₂ is deuterium. In some embodiments, R₂ is optionally substituted C₁-C₆ alkyl. In some embodiments, R₂ is optionally substituted C₂-C₆ alkenyl. In some embodiments, R₂ is optionally substituted C₂-C₆ alkynyl. In some embodiments, R₂ is optionally substituted aryl or heteroaryl. In some embodiments, R₂ is halogen. In some embodiments, R₂ is optionally substituted alkoxy. In some embodiments, R₂ is nitro. In some embodiments, R₂ is cyano.

The linker (L) can be any chemical moiety that can serve to connect the detectable label and the enamide group. In some embodiments, the linker can be any linker having about 50 atoms or less, about 40 atoms or less, about 30 atoms or less, about 20 atoms or less, or about 10 atoms or less. In some embodiments, the linker (L) can be selected from the group consisting of: —O—, —S—, —S—S—, —NR^{a}—, —C(O)—, —C(O)O—, —C(O)NR^{a}—, —SO—, —SO₂—, —SO₂NR^{a}—, substituted or unsubstituted alkyl, substituted or unsubstituted alkenyl, substituted or unsubstituted alkynyl, arylalkyl, arylalkenyl, arylalkynyl, heteroarylalkyl, heteroarylalkenyl, heteroarylalkynyl, heterocyclylalkyl, heterocyclylalkenyl, heterocyclylalkynyl, aryl, heteroaryl, heterocyclyl, cycloalkyl, cycloalkenyl, alkylarylalkyl, alkylarylalkenyl, alkylarylalkynyl, alkenylarylalkyl, alkenylarylalkenyl, alkenylarylalkynyl, alkynylarylalkyl, alkynylarylalkenyl, alkynylarylalkynyl, alkylheteroarylalkyl, alkylheteroarylalkenyl, alkylheteroarylalkynyl, alkenylheteroarylalkyl, alkenylheteroarylalkenyl, alkenylheteroarylalkynyl, alkynylheteroarylalkyl, alkynylheteroarylalkenyl, alkynylheteroarylalkynyl, alkylheterocyclylalkyl, alkylheterocyclylalkenyl, alkylhererocyclylalkynyl, alkenylheterocyclylalkyl, alkenylheterocyclylalkenyl, alkenylheterocyclylalkynyl, alkynylheterocyclylalkyl, alkynylheterocyclylalkenyl, alkynylheterocyclylalkynyl, alkylaryl, alkenylaryl, alkynylaryl, alkylheteroaryl, alkenylheteroaryl, alkynylhereroaryl; wherein backbone of the linker can be interrupted or terminated by O, S, S(O), SO₂, N(R^{a})₂, C(O), C(O)O, C(O)NR^{a}, cleavable linking group, substituted or unsubstituted aryl, substituted or unsubstituted heteroaryl, substituted or unsubstituted heterocyclic, and wherein R^{a} is hydrogen, acyl, aliphatic or substituted aliphatic.

In some embodiments, the detectable label is a fluorescent molecule, and the compound corresponds to Formula II:

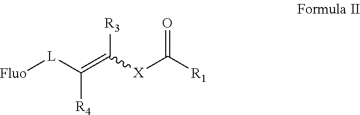

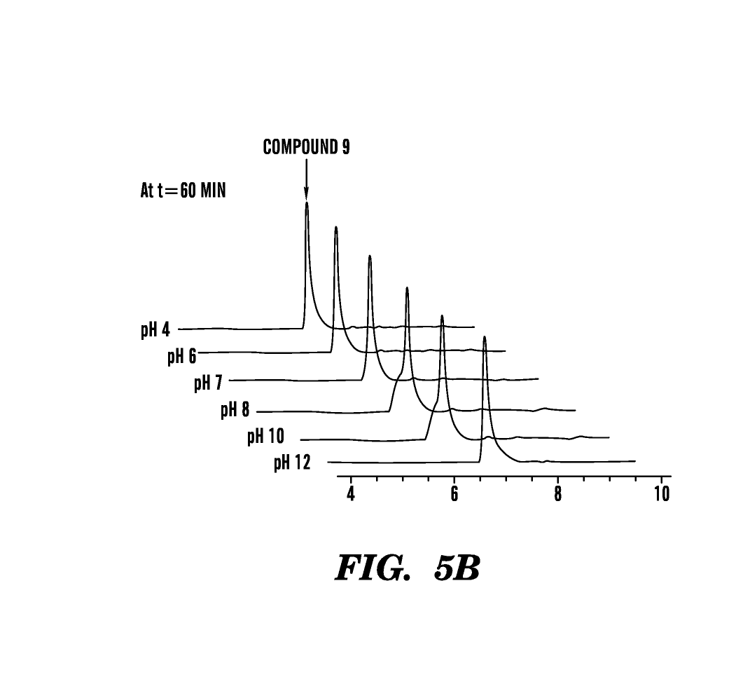

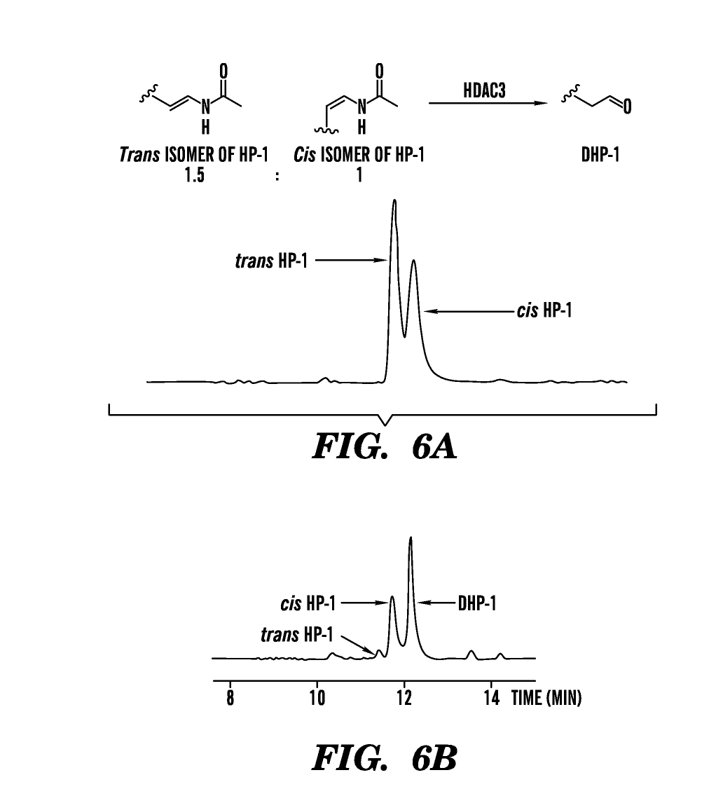

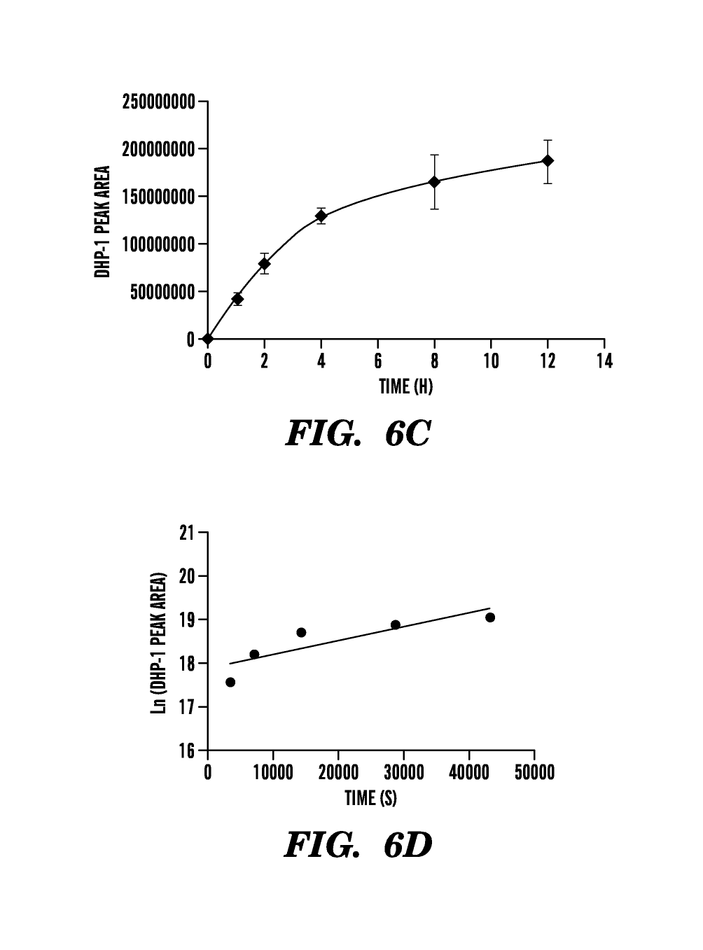

In some embodiments, the compound is a trans-isomer, a cis-isomer, or a combination thereof. A deacetylase enzyme may have isoform selectivity towards the compound. That is, the deacetylase enzyme can deacetylate a particular isomer with higher efficiency than any other isomers under the same conditions. The efficiency is at least 10% higher, at least 20% higher, at least 40% higher, at least 60% higher, at least 80% higher, at least 100% higher, at least 150% higher, at least 200% higher, at least 500% higher. Without wishing to be bound by theory, the isoform selectivity is the result of different binding affinity between the isomers and the enzyme. It would only require routine experimentation for one of ordinary skill in the art to determine the isomer choice. For example, the inventors have discovered that HDAC1 or HDAC3 selectively deacetylates the trans-isomer of HP-1 (FIG. 6).

In some embodiments, the compound is a trans-isomer.

If the deacetylase enzyme is found to not have isoform selectivity towards the compound, any isomer or a mixture of isomers can be used to detect enzyme activity.

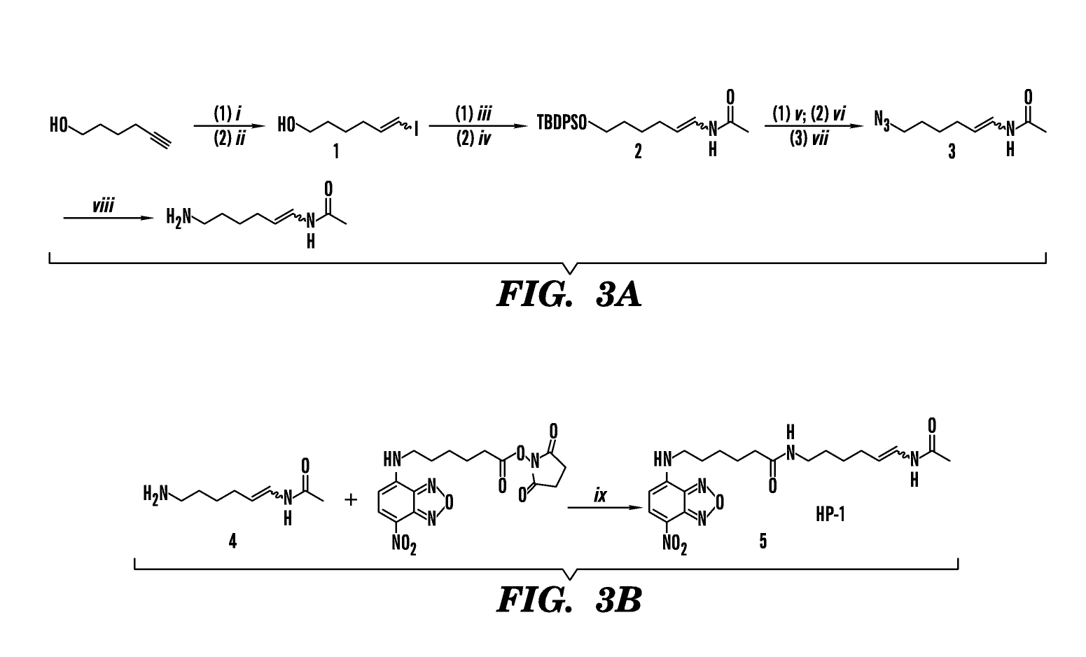

Compounds disclosed herein can be prepared beginning with commercially available starting materials and utilizing general synthetic techniques and procedures known to those skilled in the art. Chemicals may be purchased from companies such as for example Sigma-Aldrich, VWR and Alfa Aesar. Chromatography supplies and equipment may be purchased from such companies as for example Biotage AB, Charlottesville, Va.; Analytical Sales and Services, Inc., Pompton Plains, N.J.; Teledyne Isco, Lincoln, Nebr.; VWR International, Bridgeport, N.J.; Varian Inc., Palo Alto, Calif., and Mettler Toledo Instrument Newark, Del. Biotage, ISCO and Analogix columns are pre-packed silica gel columns used in standard chromatography. For example, some compounds can be synthesized using the steps or modified steps as shown in the schemes in FIGS. 3A & 3B. Exemplary synthesis of various compounds of Formula I is also described in the Examples section. Ordinarily skilled artisans can easily adapt the methods described in the Examples sections for preparing any one of the compounds of Formula I.

### Detectable Label

In some embodiments, the detectable label is an imagining agent or a contrast agent. As used herein, the term “imaging agent” refers to an element or functional group in a molecule that allows for the detection, imaging, and/or measuring enzyme activity. The imaging agent can be an echogenic substance (either liquid or gas), non-metallic isotope, an optical reporter, a boron neutron absorber, a paramagnetic metal ion, a ferromagnetic metal, a gamma-emitting radioisotope, a positron-emitting radioisotope, or an x-ray absorber. As used herein, the term “contrast agent” refers to an element or functional group in a molecule that changes the optical properties of tissue or organ containing the molecule. Optical properties that can be changed include, but are not limited to, absorbance, reflectance, fluorescence, birefringence, optical scattering and the like.

In some embodiments, the detectable label can be an optical reporter, non-metallic isotope, a paramagnetic metal ion, a ferromagnetic metal, echogenic substance (either liquid or gas), a boron neutron absorber, a gamma-emitting radioisotope, a positron-emitting radioisotope, or an x-ray absorber. Suitable non-metallic isotopes include, but are not limited to, ¹¹C, ¹⁴C, ¹³N, ¹⁸F, ¹²³I, ¹²⁴I, and ¹²⁵I. Suitable echogenic gases include, but are not limited to, a sulfur hexafluoride or perfluorocarbon gas, such as perfluoromethane, perfluoroethane, perfluoropropane, perfluorobutane, perfluorocyclobutane, perfluropentane, or perfluorohexane. Suitable paramagnetic metal ions include, but are not limited to, Gd(III), Dy(III), Fe(III), and Mn(II). Suitable X-ray absorbers include, but are not limited to, Re, Sm, Ho, Lu, Pm, Y, Bi, Pd, Gd, La, Au, Au, Yb, Dy, Cu, Rh, Ag, and Ir.

In some embodiments, the detectable label can be a fluorescent molecule, a radioisotope, a nucleotide chromophore, an enzyme, an enzyme substrate, a chemiluminescent moiety, a magnetic particle, a bioluminescent moiety, a nucleic acid, an antibody, or any combination thereof.

In some embodiments, the fluorescent molecule is a fluorophore. Typically, a fluorophore is an aromatic or heteroaromatic compound and can be a pyrene, anthracene, naphthalene, acridine, stilbene, indole, benzindole, oxazole, thiazole, benzothiazole, cyanine, carbocyanine, salicylate, anthranilate, coumarin, fluorescein, rhodamine or other like compound. Any fluorophore can be used in the compositions described in the present invention. Examples of fluorophores include, but are not limited to, fluorescein-type fluorophores, rhodamine-type fluorophores, xanthine-typefluorophores, naphthalene-type fluorophores, carbocyanine-type fluorophores, dipyrromethene boron-type fluorophores, coumarin-type fluorophores, acridine-type fluorophores, pyrene-type fluorophores, DANSYL-type fluorophores, lanthanide chelate-type fluorophores.

Exemplary fluorophores also include, but are not limited to, 1,5 IAEDANS; 1,8-ANS; 4-Methylumbelliferone; 5-carboxy-2,7-dichlorofluorescein; 5-Carboxyfluorescein (5-FAM); 5-Carboxynapthofluorescein (pH 10); 5-Carboxytetramethylrhodamine (5-TAMRA); 5-FAM (5-Carboxyfluorescein); 5-Hydroxy Tryptamine (HAT); 5-ROX (carboxy-X-rhodamine); 5-TAMRA (5-Carboxytetramethylrhodamine); 6-Carboxyrhodamine 6G; 6-CR 6G; 6-JOE; 7-Amino-4-methylcoumarin; 7-Aminoactinomycin D (7-AAD); 7-Hydroxy-4-methylcoumarin; 9-Amino-6-chloro-2-methoxyacridine; ABQ; Acid Fuchsin; ACMA (9-Amino-6-chloro-2-methoxyacridine); Acridine Orange; Acridine Red; Acridine Yellow; Acriflavin; Acriflavin Feulgen SITSA; Aequorin (Photoprotein); Alexa Fluor 350™; Alexa Fluor 430™; Alexa Fluor 488™; Alexa Fluor 532™; Alexa Fluor 546™; Alexa Fluor 568™; Alexa Fluor 594™; Alexa Fluor 633™; Alexa Fluor 647™; Alexa Fluor 660™; Alexa Fluor 680™; Alizarin Complexon; Alizarin Red; Allophycocyanin (APC); AMC, AMCA-S; AMCA (Aminomethylcoumarin); AMCA-X; Aminoactinomycin D; Aminocoumarin; Anilin Blue; Anthrocyl stearate; APC-Cy7; APTS; Astrazon Brilliant Red 4G; Astrazon Orange R; Astrazon Red 6B; Astrazon Yellow 7 GLL; Atabrine; ATTO-TAG™ CBQCA; ATTO-TAG™ FQ; Auramine; Aurophosphine G; Aurophosphine; BAO 9 (Bisaminophenyloxadiazole); BCECF (high pH); BCECF (low pH); Berberine Sulphate; Beta Lactamase; BFP blue shifted GFP (Y66H); BG-647; Bimane; Bisbenzamide; Blancophor FFG; Blancophor SV; BOBO™-1; BOBO™-3; Bodipy 492/515; Bodipy 493/503; Bodipy 500/510; Bodipy 505/515; Bodipy 530/550; Bodipy 542/563; Bodipy 558/568; Bodipy 564/570; Bodipy 576/589; Bodipy 581/591; Bodipy 630/650-X; Bodipy 650/665-X; Bodipy 665/676; Bodipy Fl; Bodipy FL ATP; Bodipy Fl-Ceramide; Bodipy R6G SE; Bodipy TMR; Bodipy TMR-X conjugate; Bodipy TMR-X, SE; Bodipy TR; Bodipy TR ATP; Bodipy TR-X SE; BO-PRO™-1; BO-PRO™-3; Brilliant Sulphoflavin FF; Calcein; Calcein Blue; Calcium Crimson™; Calcium Green; Calcium Green-1 Ca2+ Dye; Calcium Green-2 Ca2+; Calcium Green-5N Ca2+; Calcium Green-C18 Ca2+; Calcium Orange; Calcofluor White; Carboxy-X-rhodamine (5-ROX); Cascade Blue™; Cascade Yellow; Catecholamine; CFDA; CFP—Cyan Fluorescent Protein; Chlorophyll; Chromomycin A; Chromomycin A; CMFDA; Coelenterazine; Coelenterazine cp; Coelenterazine f; Coelenterazine fcp; Coelenterazine h; Coelenterazine hcp; Coelenterazine ip; Coelenterazine O; Coumarin Phalloidin; CPM Methylcoumarin; CTC; Cy2™; Cy3.1 8; Cy3.5™; Cy3™; Cy5.1 8; Cy5.5™; Cy5™; Cy7™; Cyan GFP; cyclic AMP Fluorosensor (FiCRhR); d2; Dabcyl; Dansyl; Dansyl Amine; Dansyl Cadaverine; Dansyl Chloride; Dansyl DHPE; Dansyl fluoride; DAPI; Dapoxyl; Dapoxyl 2; Dapoxyl 3; DCFDA; DCFH (Dichlorodihydrofluorescein Diacetate); DDAO; DHR (Dihydorhodamine 123); Di-4-ANEPPS; Di-8-ANEPPS (non-ratio); DiA (4-Di-16-ASP); DIDS; Dihydorhodamine 123 (DHR); DiO (DiOC18(3)); DiR; DiR (DiIC18(7)); Dopamine; DsRed; DTAF; DY-630-NHS; DY-635-NHS; EBFP; ECFP; EGFP; ELF 97; Eosin; Erythrosin; Erythrosin ITC; Ethidium homodimer-1 (EthD-1); Euchrysin; Europium (III) chloride; Europium; EYFP; Fast Blue; FDA; Feulgen (Pararosaniline); FITC; FL-645; Flazo Orange; Fluo-3; Fluo-4; Fluorescein Diacetate; Fluoro-Emerald; Fluoro-Gold (Hydroxystilbamidine); Fluor-Ruby; FluorX; FM 1-43™; FM 4-46; Fura Red™ (high pH); Fura-2, high calcium; Fura-2, low calcium; Genacryl Brilliant Red B; Genacryl Brilliant Yellow 10GF; Genacryl Pink 3G; Genacryl Yellow 5GF; GFP (S65T); GFP red shifted (rsGFP); GFP wild type, non-UV excitation (wtGFP); GFP wild type, UV excitation (wtGFP); GFPuv; Gloxalic Acid; Granular Blue; Haematoporphyrin; Hoechst 33258; Hoechst 33342; Hoechst 34580; HPTS; Hydroxycoumarin; Hydroxystilbamidine (FluoroGold); Hydroxytryptamine; Indodicarbocyanine (DiD); Indotricarbocyanine (DiR); Intrawhite Cf; JC-1; JO-JO-1; JO-PRO-1; LaserPro; Laurodan; LDS 751; Leucophor PAF; Leucophor SF; Leucophor WS; Lissamine Rhodamine; Lissamine Rhodamine B; LOLO-1; LO-PRO-1; Lucifer Yellow; Mag Green; Magdala Red (Phloxin B); Magnesium Green; Magnesium Orange; Malachite Green; Marina Blue; Maxilon Brilliant Flavin 10 GFF; Maxilon Brilliant Flavin 8 GFF; Merocyanin; Methoxycoumarin; Mitotracker Green FM; Mitotracker Orange; Mitotracker Red; Mitramycin; Monobromobimane; Monobromobimane (mBBr-GSH); Monochlorobimane; MPS (Methyl Green Pyronine Stilbene); NBD; NBD Amine; Nile Red; Nitrobenzoxadiazole (NBD); Noradrenaline; Nuclear Fast Red; Nuclear Yellow; Nylosan Brilliant Iavin E8G; Oregon Green™; Oregon Green 488-X; Oregon Green™ 488; Oregon Green™ 500; Oregon Green™ 514; Pacific Blue; Pararosaniline (Feulgen); PE-Cy5; PE-Cy7; PerCP; PerCP-Cy5.5; PE-TexasRed (Red 613); Phloxin B (Magdala Red); Phorwite AR; Phorwite BKL; Phorwite Rev; Phorwite RPA; Phosphine 3R; PhotoResist; Phycoerythrin B [PE]; Phycoerythrin R [PE]; PKH26 ; PKH67; PMIA; Pontochrome Blue Black; POPO-1; POPO-3; PO-PRO-1; PO-PRO-3; Primuline; Procion Yellow; Propidium Iodid (PI); PyMPO; Pyrene; Pyronine; Pyronine B; Pyrozal Brilliant Flavin 7GF; QSY 7; Quinacrine Mustard; Resorufin; RH 414; Rhod-2; Rhodamine; Rhodamine 110; Rhodamine 123; Rhodamine 5 GLD; Rhodamine 6G; Rhodamine B 540; Rhodamine B 200; Rhodamine B extra; Rhodamine BB; Rhodamine BG; Rhodamine Green; Rhodamine Phallicidine; Rhodamine Phalloidine; Rhodamine Red; Rhodamine WT; Rose Bengal; R-phycoerythrin (PE); red shifted GFP (rsGFP, S65T); S65A; S65C; S65L; S65T; Sapphire GFP; Serotonin; Sevron Brilliant Red 2B; Sevron Brilliant Red 4G; Sevron Brilliant Red B; Sevron Orange; Sevron Yellow L; sgBFP™; sgBFP™ (super glow BFP); sgGFP™ sgGFP™ (super glow GFP); SITS; SITS (Primuline); SITS (Stilbene Isothiosulphonic Acid); SPQ (6-methoxy-N-(3-sulfopropyl)-quinolinium); Stilbene; Sulphorhodamine B can C; Sulphorhodamine G Extra; Tetracycline; Tetramethylrhodamine; Texas Red™; Texas Red-X™ conjugate; Thiadicarbocyanine (DiSC3); Thiazine Red R; Thiazole Orange; Thioflavin 5; Thioflavin S; Thioflavin TCN; Thiolyte; Thiozole Orange; Tinopol CBS (Calcofluor White); TMR; TO-PRO-1; TO-PRO-3; TO-PRO-5; TOTO-1; TOTO-3; TriColor (PE-Cy5); TRITC (TetramethylRodamineIsoThioCyanate); True Blue; TruRed; Ultralite; Uranine B; Uvitex SFC; wt GFP; WW 781; XL665; X-Rhodamine; XRITC; Xylene Orange; Y66F; Y66H; Y66W; Yellow GFP; YFP; YO-PRO-1; YO-PRO-3; YOYO-1; and YOYO-3. Many suitable forms of these fluorescent compounds are available and can be used.

In some embodiments, the fluorophore comprises hydroxycoumarin, aminocoumarin, methoxycoumarin, cascade blue, pacific blue, pacific orange, lucifer yellow, nitrobenzoxadiazole (NBD), R-phycoerythrin, PE-Cy5 conjugates, PE-Cy7 conjugates, Red 613, PerCP, TruRed, FluorX, Fluorescein, BODIPY, Cy2, Cy3, Cy3B, Cy3.5, Cy5, Cy5.5, Cy7, SeTau-647, TRITC, rhodamine, Texas Red, allophycocyanin (APC), APC-Cy7 conjugates, or derivatives thereof.

In some embodiments, the fluorophore is NBD, and the compound corresponds to Formula III:

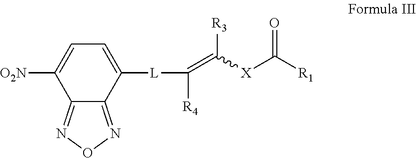

In some embodiments, the compound corresponds to Formula IV:

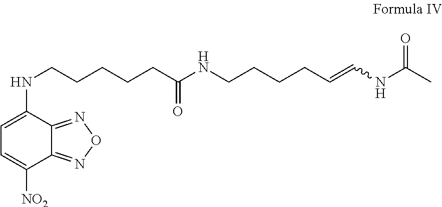 [SMILES: CC(=O)NC=CCCCCNC(=O)CCCCCNc1ccc([N+](=O)[O-])c2nonc12]

In some embodiments, the fluorescent molecule is a fluorescent nanoparticle that includes, but is not limited to, a quantum dot, a metallic nanoparticle, or a nanodiamond.

In general, a fluorescent molecule should have favorable excitation and emission wavelengths, and as a result be excitable and detectable by readily available light sources and detectors. Furthermore, the fluorescent molecule should have a high quantum yield and/or high molar absorption coefficient.

In some embodiments, the detectable label is a magnetic resonance imaging (MRI) contrast agent. Any MRI contrast agent can be used in the compositions described in the present invention. A contrast agent is often used in conjunction with MRI to improve and/or enhance the images obtained of a person's body. A contrast agent is a chemical substance that is introduced into the body to change the contrast between two tissues. Generally, MRI contrast agents comprise magnetic probes that are designed to enhance a given image by affecting the proton relaxation rate of the water molecules in proximity to the MRI contrast agent. This selective change of the T₁ (Spin-Lattice Relaxation Time) and T₂ (Spin-Spin Relaxation Time) of the tissues in the vicinity of the MRI contrast agents changes the contrast of the tissues visible via MRI.

Typical MRI contrast agents belong to one of two classes: (1) complexes of a paramagnetic metal ion, such as gadolinium (Gd), or (2) coated iron nanoparticles. As free metal ions are toxic to the body, they are typically complexed with other molecules or ions to prevent them from complexing with molecules in the body, thereby lessening their toxicity. Some typical MRI contrast agents include, but are not limited to: Gd-EDTA, Gd-DTPA, Gd-DOTA, Gd-BOPTA, Gd-DOPTA, Gd-DTPA-BMA (gadodiamide), ferumoxsil, ferumoxide and ferumoxtran. Gd chelated contrast agents approved by the U.S. Food and Drug Administration (FDA) include, but are not limited to, gadoterate (Dotarem), gadodiamide (Omniscan), gadobenate (MultiHance), gadopentetate (Magnevist), gadoteridol (ProHance), gadofosveset (Ablavar, formerly Vasovist), gadoversetamide (OptiMARK), gadoxetate (Eovist), gadobutrol (Gadavist). Protein-based MRI contrast agents are also contemplated for this invention.

Another class of MRI contrast agents—called “smart” contrast agents—includes contrast agents that are activated by the physiology of the body or a property of a tumor, i.e., agents that are activated by pH, temperature and/or the presence of certain enzymes or ions. Some examples of MRI smart contrast agents include, but are not limited to, contrast agents that are sensitive to the calcium concentration in a body, or those that are sensitive to pH.

More examples of MRI contrast agents can be found in “The Chemistry of Contrast Agents in Medical Magnetic Resonance Imaging” by Merbach et al. (Wiley; 2 edition, Apr. 15, 2013).

In some embodiments, the detectable label is a radioisotope. Radioisotopes are commonly used in medicine to provide diagnostic information about the functioning of a person's specific organs, or to treat them. Diagnostic procedures using radioisotopes are now routine. Once placed in the body, radioisotopes can emit signals in the form of gamma rays from within the body. Examples of radioisotopes can include, but are not limited to, ¹¹C, ¹³N, ¹⁵O, ¹³O, ¹²⁴I, ¹²³I, ¹⁸F, ⁶⁶Ga, ⁶⁸Ga, ⁴⁴Sc, ⁷²As, ⁶⁰Cu, ⁶¹Cu, ⁶²Cu, ⁶⁴Cu, ¹⁹⁸Pb, ¹⁹⁷Hg, ⁹⁷Ru, ⁵²Fe, ⁵⁵Co, ⁸²Rb, ⁸²Sr, ⁶⁸Ge, ⁸⁹Zr, ⁸⁶Y, ⁹⁹mTc, ¹¹¹In, ¹²⁵I, ⁴⁴Ti, ²⁰³Pb, ²⁰¹Tl, ⁶⁷Cu and ⁶⁷Ga. Such isotopes are particularly useful for PET (positron emission tomography) or SPECT (single photon emission computed tomography). Other non-limiting examples of radioisotopes include yttrium (⁹⁰Y), lutetium (¹⁷⁷Lu), actinium (²²⁵Ac), praseodymium, astatine (²¹¹At), rhenium (¹⁸⁶Re), bismuth (²¹²Bi or ²¹³Bi), holmium (¹⁶⁶Ho), samarium (¹⁵³Sm), iridium (¹⁹²Ir), rhodium ¹⁰⁵Rh), iodine (¹³¹I or ¹²⁵I), indium (¹¹¹In), technetium (⁹⁹Tc), phosphorus (³²P), sulfur (³⁵S), carbon (¹⁴C), tritium (³H), chromium (⁵¹Cr), chlorine (³¹Cl), cobalt (⁵⁷Co or ⁵⁸Co), iron (⁵⁹Fe), selenium (⁷⁵Se) and gallium (⁶⁷Ga). More examples of radioisotopes can be found in “Essentials of Nuclear Medicine Imaging: Expert Consult—Online and Print” by Mettler and Guiberteau (Saunders; 6 edition, Jan. 25, 2012).

In some embodiments, a radioisotope can be bound to a chelating agent. Suitable radioisotopes for direct conjugation include, without limitation, 18F, 124I, 125I, 131I, and mixtures thereof. Suitable radioisotopes for use with a chelating agent include, without limitation, 47Sc, 64Cu, 67Cu, 89Sr, 86Y, 87Y, 90Y, 105Rh, 111Ag, 111In, 117m5n, 149Pm, 153Sm, 166Ho, 177Lu, 186Re, 188Re, 211At, 212Bi, and mixtures thereof. Suitable chelating agents include, but are not limited to, DOTA, BAD, TETA, DTPA, EDTA, NTA, HDTA, their phosphonate analogs, and mixtures thereof.

Other exemplary detectable labels include luminescent and bioluminescent markers (e.g., biotin, luciferase (e.g., bacterial, firefly, click beetle and the like), luciferin, and aequorin), radiolabels (e.g., 3H, 125I, 35S, 14C, or 32P), enzymes (e.g., galactosidases, glucorinidases, phosphatases (e.g., alkaline phosphatase), peroxidases (e.g., horseradish peroxidase), and cholinesterases), and calorimetric labels such as colloidal gold or colored glass or plastic (e.g., polystyrene, polypropylene, and latex) beads. Patents teaching the use of such labels include U.S. Pat. Nos. 3,817,837, 3,850,752, 3,939,350, 3,996,345, 4,277,437, 4,275,149, and 4,366,241, each of which is incorporated herein by reference.

Means of detecting such labels are well known to those of skill in the art. Thus, for example, radiolabels can be detected using photographic film or scintillation counters, fluorescent markers can be detected using a photo-detector to detect emitted light. Enzymatic labels are typically detected by providing the enzyme with an enzyme substrate and detecting the reaction product produced by the action of the enzyme on the enzyme substrate, and calorimetric labels can be detected by visualizing the colored label. Exemplary methods for in vivo detection or imaging of detectable labels include, but are not limited to, radiography, magnetic resonance imaging (MRI), Positron emission tomography (PET), Single-photon emission computed tomography (SPECT, or less commonly, SPET), Scintigraphy, ultrasound, CAT scan, photoacoustic imaging, thermography, linear tomography, poly tomography, zonography, orthopantomography (OPT or OPG), and computed Tomography (CT) or Computed Axial Tomography (CAT scan).

### Enzyme Activity Detection

In one aspect, a method for detecting the enzyme activity of a deacetylase enzyme is provided. The method comprises contacting the deacetylase enzyme with a compound described herein, and determining the deacetylase activity by measuring a signal produced by a fragment of the compound.

In some embodiments, the contacting is in vivo. In some embodiments, the contacting is ex vivo.

In some embodiments of in vivo contacting, the method further comprises administering the compound to a subject. In some embodiments, the compound is administered in a pharmaceutically-acceptable carrier. In some embodiments, the subject is a mammal. In some embodiments, the subject is a human.

In some embodiments, the method further comprises incubating the compound with the deacetylase enzyme for a period of time, such as at least a minute, at least 5 minutes, at least 10 minutes, at least 20 minutes, at least 40 minutes, at least an hour, at least two hours, at least four hours, or at least six hours. The incubation period can permit the deacetylase enzyme to complete the cleaving process. Depending on the particular compound used for enzyme activity detection, the reaction rate can vary. A skilled artisan can easily determine the incubation period based on factors such as the reaction rate.

When a deacetylase enzyme cleaves a compound described herein, a nucleophilic fragment comprising the detectable label is produced thereafter. Without wishing to be bound by theory, in some embodiments of which the deacetylase enzyme is within a cell, the fragment can be localized within the cell through non-specific interaction with the nucleophilic portion of the fragment. In some embodiments of which the deacetylase enzyme is within a cell, the method further comprises, after the incubation period, washing the cell to remove any unreacted compound.

In some embodiments, the deacetylase enzyme is extracted from a cell or a population of cells. In these embodiments, the nucleophilic fragment comprising the detectable label can be separated and collected by methods such as column chromatography.

Detection and quantification of a signal produced by the nucleophilic fragment can thus be used to measure enzyme activity. The intensity of the signal measured is proportional to the degree of enzyme activity.

In some embodiments, the methods described herein can be used to locate increased deacetylase enzyme activity in vivo.

### Screening

In another aspect, provided herein are methods for screening for substances that modulate activity of deacetylase enzymes, the methods comprising (i) contacting the substance with a deacetylase enzyme; (ii) contacting the deacetylase enzyme with a compound described herein; and (iii) determining the effect of the substance on deacetylase enzyme activity by measuring and comparing a signal produced by a fragment of the compound relative to a control, wherein the control is performed in the absence of the substance. A substance is considered to enhance deacetylase enzyme activity if the detected signal is above a reference level determined from the control. Similarly, a substance is considered to reduce deacetylase enzyme activity if the detected signal is below a reference level determined from the control. In some embodiments, the methods described herein can be used to identify an agent that reduces or enhances deacetylase activity by at least about 10%, 25%, 50%, 60%, 70%, 80%, 90%, or 100%, or more, relative to the absence of the agent.

In some embodiments, the methods described herein can be used for screening HDAC inhibitors.

The substances for screening can be naturally occurring or synthesized in the laboratory. Typical substances for screening include, but are not limited to, small organic or inorganic molecules, proteins, peptides, polynucleotides, polynucleotide analogs, peptide analogs, lipids, and carbohydrates. They can be isolated from microorganisms, animals, or plants, and can be produced recombinantly, or synthesized by chemical methods known in the art. If desired, substances or test agents can be obtained using any of the numerous combinatorial library methods known in the art, including but not limited to, biological libraries, spatially addressable parallel solid phase or solution phase libraries, synthetic library methods requiring deconvolution, the “one-bead one-compound” library method, and synthetic library methods using affinity chromatography selection.

Substances can be screened for the ability to modulate deacetylase activity using high throughput screening. Using high throughput screening, many substances can be tested in parallel so that large numbers of substances can be quickly screened. The most widely established techniques utilize 96-well microtiter plates. In addition to the plates, many instruments, materials, pipettors, robotics, plate washers, and plate readers are commercially available to fit the 96-well format.

The screening can be performed either in vitro or in vivo.

In yet another aspect, the invention provides a method for targeting a cell comprising a deacetylase enzyme within a cell population, the method comprising contacting the cell population with a compound that includes an enamide group (e.g., the compounds described herein). For example, the compound can be a molecular cargo conjugated to an enamide group. The molecular cargo can be small chemical molecules, peptides, protein, DNA, RNA such as siRNA and miRNA, or nanosize particles. In some embodiments, the molecular cargo is a drug.

As described previously, the compound can be enzymatically cleaved by the deacetylase enzyme within the cell and the cargo can then be retained in the cell. For cells that do not comprise the deacetylase enzyme, little or no cargo would be retained in these cells.

It should be understood that this invention is not limited to the particular methodology, protocols, and reagents, etc., described herein and as such may vary. The terminology used herein is for the purpose of describing particular embodiments only, and is not intended to limit the scope of the present invention, which is defined solely by the claims.

As used herein and in the claims, the singular forms include the plural reference and vice versa unless the context clearly indicates otherwise. Other than in the operating examples, or where otherwise indicated, all numbers expressing quantities of ingredients or reaction conditions used herein should be understood as modified in all instances by the term “about.”

All patents and other publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

Although any known methods, devices, and materials may be used in the practice or testing of the invention, the methods, devices, and materials in this regard are described herein.

Some embodiments of the invention are listed in the following paragraphs:
1. A compound characterized in having a structure: Lab-L-Ena, wherein Lab is a detectable label, L is a linker, and Ena is an enamide group.
2. The compound of paragraph 1, corresponding to Formula I.
3. The compound of paragraph 1 or 2, wherein the detectable label is an imagining agent or a contrast agent.
4. The compound of any of paragraphs 1-3, wherein the detectable label is selected from a group consisting of an optical reporter, non-metallic isotope, a paramagnetic metal ion, a ferromagnetic metal, echogenic substance (either liquid or gas), a boron neutron absorber, a gamma-emitting radioisotope, a positron-emitting radioisotope, and an x-ray absorber.
5. The compound of any of paragraphs 1-4, wherein the detectable label is selected from a group consisting of fluorescent molecules, radioisotopes, nucleotide chromophores, enzymes, enzyme substrates, chemiluminescent moieties, magnetic particles, bioluminescent moieties, nucleic acids, antibodies, and any combinations thereof.
6. The compound of paragraph 5, corresponding to Formula II.
7. The compound of paragraph 5 or 6, wherein the fluorescent molecule comprises hydroxycoumarin, aminocoumarin, methoxycoumarin, cascade blue, pacific blue, pacific orange, lucifer yellow, nitrobenzoxadiazole (NBD), R-phycoerythrin, PE-Cy5 conjugates, PE-Cy7 conjugates, Red 613, PerCP, TruRed, FluorX, Fluorescein, BODIPY, Cy2, Cy3, Cy3B, Cy3.5, Cy5, Cy5.5, Cy7, SeTau-647, TRITC, rhodamine, Texas Red, allophycocyanin (APC), APC-Cy7 conjugates, or derivatives thereof.
8. The compound of paragraph 7, wherein the fluorescent molecule comprises NBD, and wherein the compound corresponds to Formula III.
9. The compound of any of paragraphs 1-8, wherein the linker is selected from the group consisting of: —O—, —S—, —S—S—, —NR^{a}—, —C(O)—, —C(O)O—, —C(O)NR^{a}—, —SO—, —SO₂—, —SO₂NR^{a}—, substituted or unsubstituted alkyl, substituted or unsubstituted alkenyl, substituted or unsubstituted alkynyl, arylalkyl, arylalkenyl, arylalkynyl, heteroarylalkyl, heteroarylalkenyl, heteroarylalkynyl, heterocyclylalkyl, heterocyclylalkenyl, heterocyclylalkynyl, aryl, heteroaryl, heterocyclyl, cycloalkyl, cycloalkenyl, alkylarylalkyl, alkylarylalkenyl, alkylarylalkynyl, alkenylarylalkyl, alkenylarylalkenyl, alkenylarylalkynyl, alkynylarylalkyl, alkynylarylalkenyl, alkynylarylalkynyl, alkylheteroarylalkyl, alkylheteroarylalkenyl, alkylheteroarylalkynyl, alkenylheteroarylalkyl, alkenylheteroarylalkenyl, alkenylheteroarylalkynyl, alkynylheteroarylalkyl, alkynylheteroarylalkenyl, alkynylheteroarylalkynyl, alkylheterocyclylalkyl, alkylheterocyclylalkenyl, alkylhererocyclylalkynyl, alkenylheterocyclylalkyl, alkenylheterocyclylalkenyl, alkenylheterocyclylalkynyl, alkynylheterocyclylalkyl, alkynylheterocyclylalkenyl, alkynylheterocyclylalkynyl, alkylaryl, alkenylaryl, alkynylaryl, alkylheteroaryl, alkenylheteroaryl, alkynylhereroaryl; wherein backbone of the linker can be interrupted or terminated by O, S, S(O), SO₂, N(R^{a})₂, C(O), C(O)O, C(O)NR^{a}, cleavable linking group, substituted or unsubstituted aryl, substituted or unsubstituted heteroaryl, substituted or unsubstituted heterocyclic, and wherein R^{a} is hydrogen, acyl, aliphatic or substituted aliphatic.
10. The compound of paragraph 8 or 9, corresponding to Formula IV.
11. The compound of any of paragraphs 1-10, wherein the compound is a trans-isomer.
12. A method of detecting enzyme activity of a deacetylase enzyme, the method comprising
(i) contacting the deacetylase enzyme with a compound of any of paragraphs 1-11; and
(ii) determining the deacetylase activity by measuring a signal produced by a fragment of the compound.
13. The method of paragraph 12, wherein the deacetylase enzyme is a histone deacetylase (HDAC) or a sirtuin.
14. The method of paragraph 13, wherein the deacetylase enzyme is one of Class I HDAC enzymes.
15. The method of paragraph 14, wherein the deacetylase enzyme is HDAC1, HDAC3, or a combination thereof.
16. The method of any of paragraphs 12-15, wherein the signal is a fluorescent signal, a magnetic signal, or a radioactive signal.
17. The method of any of paragraphs 12-16, wherein the fragment of the compound is produced by the deacetylase enzyme cleaving the compound.
18. The method of any of paragraphs 12-17, wherein the contacting is ex vivo.
19. The method of any of paragraphs 12-17, wherein the contacting is in vivo.
20. The method of any of paragraphs 12-19, wherein the deacetylase enzyme is within a cell, and wherein the signal is localized within the cell.
21. The method of paragraph 20, further comprising administering the compound to a subject comprising the cell.
22. The method of paragraph 21, wherein the compound is administered in a pharmaceutically-acceptable carrier.
23. The method of paragraph 21 or 22, wherein the subject is a mammal.
24. The method of paragraph 23, wherein the mammal is a human.
25. A method of screening a substance for its effect on deacetylase enzyme activity, the method comprising:
(i) contacting the substance with a deacetylase enzyme;
(ii) contacting the deacetylase enzyme with a compound of any of paragraphs 1-11; and
(iii) determining the effect of the substance on deacetylase enzyme activity by measuring and comparing a signal produced by a fragment of the compound relative to a control, wherein the control is performed in the absence of the substance.
26. The method of paragraph 25, wherein the deacetylase enzyme is a histone deacetylase (HDAC) or a sirtuin.
27. The method of paragraph 26, wherein the deacetylase enzyme is one of Class I HDAC enzymes.
28. The method of paragraph 27, wherein the deacetylase enzyme is HDAC1, HDAC3, or a combination thereof.
29. The method of any of paragraphs 25-28, wherein the signal is a fluorescent signal, a magnetic signal, or a radioactive signal.
30. The method of any of paragraphs 25-29, wherein the fragment of the compound is produced by the deacetylase enzyme cleaving the compound.
31. The method of any of paragraphs 25-30, wherein the substance enhances deacetylase enzyme activity if the signal is above a reference level determined from the control.
32. The method of any of paragraphs 25-30, wherein the substance reduces deacetylase enzyme activity if the signal is below a reference level determined from the control.
33. The method of any of paragraphs 25-32, wherein the deacetylase enzyme is within a cell.
34. Use of a compound of any of paragraphs 1-11 to detect deacetylase enzyme activity.
35. A method of targeting a cell comprising a deacetylase enzyme within a cell population, the method comprising contacting the cell population with a compound of any of paragraphs 1-11.
36. A method of delivering a drug to a cell comprising a deacetylase enzyme, the method comprising contacting the cell with a composition comprising the drug linked to an enamide group.
37. The method of paragraph 35 or 36, wherein the deacetylase enzyme is a histone deacetylase (HDAC) or a sirtuin.
38. The method of paragraph 37, wherein the deacetylase enzyme is one of Class I HDAC enzymes.
39. The method of paragraph 38, wherein the deacetylase enzyme is HDAC1, HDAC3, or a combination thereof.
40. A method of forming a nucleophile, the method comprising contacting a deacetylase enzyme with a compound of any of paragraphs 1-11, whereby the deacetylase enzyme cleaves the compound to form the nucleophile.

### Definitions

Unless stated otherwise, or implicit from context, the following terms and phrases include the meanings provided below. Unless explicitly stated otherwise, or apparent from context, the terms and phrases below do not exclude the meaning that the term or phrase has acquired in the art to which it pertains. The definitions are provided to aid in describing particular embodiments, and are not intended to limit the claimed invention, because the scope of the invention is limited only by the claims. Further, unless otherwise required by context, singular terms shall include pluralities and plural terms shall include the singular.

As used herein the term “comprising” or “comprises” is used in reference to compositions, methods, and respective component(s) thereof, that are useful to an embodiment, yet open to the inclusion of unspecified elements, whether useful or not.

As used herein the term “consisting essentially of” refers to those elements required for a given embodiment. The term permits the presence of elements that do not materially affect the basic and novel or functional characteristic(s) of that embodiment of the invention.

Certain compounds of the present invention and definitions of specific functional groups are also described in more detail below. For purposes of this invention, the chemical elements are identified in accordance with the Periodic Table of the Elements, CAS version, Handbook of Chemistry and Physics, 75th Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito: 1999, the entire contents of which are incorporated herein by reference.

As used herein, the term “aliphatic” means a moiety characterized by a straight or branched chain arrangement of constituent carbon atoms and can be saturated or partially unsaturated with one or more (e.g., one, two, three, four, five or more) double or triple bonds.

As used herein, the term “alicyclic” means a moiety comprising a nonaromatic ring structure. Alicyclic moieties can be saturated or partially unsaturated with one or more double or triple bonds. Alicyclic moieties can also optionally comprise heteroatoms such as nitrogen, oxygen and sulfur. The nitrogen atoms can be optionally quaternerized or oxidized and the sulfur atoms can be optionally oxidized. Examples of alicyclic moieties include, but are not limited to moieties with C₃-C₈ rings such as cyclopropyl, cyclohexane, cyclopentane, cyclopentene, cyclopentadiene, cyclohexane, cyclohexene, cyclohexadiene, cycloheptane, cycloheptene, cycloheptadiene, cyclooctane, cyclooctene, and cyclooctadiene.

As used herein, the term “alkyl” means a straight or branched, saturated aliphatic radical having a chain of carbon atoms. Cₓ alkyl and Cₓ-C_{y} alkyl are typically used where X and Y indicate the number of carbon atoms in the chain. For example, Cₗ-C₆ alkyl includes alkyls that have a chain of between 1 and 6 carbons (e.g., methyl, ethyl, propyl, isopropyl, butyl, sec-butyl, isobutyl, tert-butyl, pentyl, neopentyl, hexyl, and the like). Alkyl represented along with another radical (e.g., as in arylalkyl) means a straight or branched, saturated alkyl divalent radical having the number of atoms indicated or when no atoms are indicated means a bond, e.g., (C₆-C₁₀)aryl(C₀-C₃)alkyl includes phenyl, benzyl, phenethyl, 1-phenylethyl 3-phenylpropyl, and the like. Backbone of the alkyl can be optionally inserted with one or more heteroatoms, such as N, O, or S.

In preferred embodiments, a straight chain or branched chain alkyl has 30 or fewer carbon atoms in its backbone (e.g., C1-C30 for straight chains, C3-C30 for branched chains), and more preferably 20 or fewer. Likewise, preferred cycloalkyls have from 3-10 carbon atoms in their ring structure, and more preferably have 5, 6 or 7 carbons in the ring structure. The term “alkyl” (or “lower alkyl”) as used throughout the specification, examples, and claims is intended to include both “unsubstituted alkyls” and “substituted alkyls”, the latter of which refers to alkyl moieties having one or more substituents replacing a hydrogen on one or more carbons of the hydrocarbon backbone. In some embodiments, a straight chain or branched chain alkyl has 5 or fewer carbon atoms, 10 or fewer carbon atoms, or 15 or fewer carbon atoms.

Unless the number of carbons is otherwise specified, “lower alkyl” as used herein means an alkyl group, as defined above, but having from one to ten carbons, more preferably from one to six carbon atoms in its backbone structure. Likewise, “lower alkenyl” and “lower alkynyl” have similar chain lengths. Throughout the application, preferred alkyl groups are lower alkyls. In preferred embodiments, a substituent designated herein as alkyl is a lower alkyl.

Substituents of a substituted alkyl can include halogen, hydroxy, nitro, thiols, amino, azido, imino, amido, phosphoryl (including phosphonate and phosphinate), sulfonyl (including sulfate, sulfonamido, sulfamoyl and sulfonate), and silyl groups, as well as ethers, alkylthios, carbonyls (including ketones, aldehydes, carboxylates, and esters), —CF₃, —CN and the like. In some embodiments, the substituted alkyl is a perfluorinated alkyl.

As used herein, the term “alkenyl” refers to unsaturated straight-chain, branched-chain or cyclic hydrocarbon radicals having at least one carbon-carbon double bond. Cₓ alkenyl and Cₓ-C_{y}alkenyl are typically used where X and Y indicate the number of carbon atoms in the chain. For example, C₂-C₆alkenyl includes alkenyls that have a chain of between 1 and 6 carbons and at least one double bond, e.g., vinyl, allyl, propenyl, isopropenyl, 1-butenyl, 2-butenyl, 3-butenyl, 2-methylallyl, 1-hexenyl, 2-hexenyl, 3-hexenyl, and the like). Alkenyl represented along with another radical (e.g., as in arylalkenyl) means a straight or branched, alkenyl divalent radical having the number of atoms indicated. Backbone of the alkenyl can be optionally inserted with one or more heteroatoms, such as N, O, or S. In some embodiments, the substituted alkenyl is a perfluorinated alkenyl.

As used herein, the term “alkynyl” refers to unsaturated hydrocarbon radicals having at least one carbon-carbon triple bond. Cₓ alkynyl and Cₓ-C_{y} alkynyl are typically used where X and Y indicate the number of carbon atoms in the chain. For example, C₂-C₆alkynyl includes alkynls that have a chain of between 1 and 6 carbons and at least one triple bond, e.g., ethynyl, 1-propynyl, 2-propynyl, 1-butyryl, isopentynyl, 1,3-hexa-diyn-yl, n-hexynyl, 3-pentynyl, 1-hexen-3-ynyl and the like. Alkynyl represented along with another radical (e.g., as in arylalkynyl) means a straight or branched, alkynyl divalent radical having the number of atoms indicated. Backbone of the alkynyl can be optionally inserted with one or more heteroatoms, such as N, O, or S. In some embodiments, the substituted alkynyl is a perfluorinated alkynyl.

The terms “alkylene,” “alkenylene,” and “alkynylene” refer to divalent alkyl, alkelyne, and alkynylene” radicals. Prefixes Cₓ and Cₓ-C_{y} are typically used where X and Y indicate the number of carbon atoms in the chain. For example, C₁-C₆alkylene includes methylene, (—CH₂—), ethylene (—CH₂CH₂—), trimethylene (—CH₂CH₂CH₂—), tetramethylene (—CH₂CH₂CH₂CH₂—), 2-methyltetramethylene (—CH₂CH(CH₃)CH₂CH₂—), pentamethylene (—CH₂CH₂CH₂CH₂CH₂—) and the like).

As used herein, the term “alkylidene” means a straight or branched unsaturated, aliphatic, divalent radical having a general formula ═CRₐR_{b}. Cₓ alkylidene and Cₓ-C_{y}alkylidene are typically used where X and Y indicate the number of carbon atoms in the chain. For example, C₂-C₆alkylidene includes methylidene (═CH₂), ethylidene (═CHCH₃), isopropylidene (═C(CH₃)₂), propylidene (═CHCH₂CH₃), allylidene (═CH—CH═CH₂), and the like).

The term “heteroalkyl”, as used herein, refers to straight or branched chain, or cyclic carbon-containing radicals, or combinations thereof, containing at least one heteroatom. Suitable heteroatoms include, but are not limited to, O, N, Si, P, Se, B, and S, wherein the phosphorous and sulfur atoms are optionally oxidized, and the nitrogen heteroatom is optionally quaternized. Heteroalkyls can be substituted as defined above for alkyl groups. In some embodiments, the heteroalkyl has 5 or fewer carbon atoms, 10 or fewer carbon atoms, or 15 or fewer carbon atoms.

As used herein, the term “halogen” or “halo” refers to an atom selected from fluorine, chlorine, bromine and iodine.

A “halogen-substituted moiety” or “halo-substituted moiety”, as an isolated group or part of a larger group, means an aliphatic, alicyclic, or aromatic moiety, as described herein, substituted by one or more “halo” atoms, as such terms are defined in this application. For example, halo-substituted alkyl includes haloalkyl, dihaloalkyl, trihaloalkyl, perhaloalkyl and the like (e.g. halosubstituted (C₁-C₃)alkyl includes chloromethyl, dichloromethyl, difluoromethyl, trifluoromethyl (—CF₃), 2,2,2-trifluoroethyl, perfluoroethyl, 2,2,2-trifluoro-1,1-dichloroethyl, and the like).

The term “aryl” refers to monocyclic, bicyclic, or tricyclic fused aromatic ring system. Cₓ aryl and Cₓ-C_{y}aryl are typically used where X and Y indicate the number of carbon atoms in the ring system. An aryl group can comprise a 4-atom ring, a 5-atom ring, a 6-atom ring, a 7-atom ring, a 8-atom ring, a 9 atom ring, or more. Exemplary aryl groups include, but are not limited to, pyridinyl, pyrimidinyl, furanyl, thienyl, imidazolyl, thiazolyl, pyrazolyl, pyridazinyl, pyrazinyl, triazinyl, tetrazolyl, indolyl, benzyl, phenyl, naphthyl, anthracenyl, azulenyl, fluorenyl, indanyl, indenyl, naphthyl, phenyl, tetrahydronaphthyl, benzimidazolyl, benzofuranyl, benzothiofuranyl, benzothiophenyl, benzoxazolyl, benzoxazolinyl, benzthiazolyl, benztriazolyl, benztetrazolyl, benzisoxazolyl, benzisothiazolyl, benzimidazolinyl, carbazolyl, 4aH carbazolyl, carbolinyl, chromanyl, chromenyl, cinnolinyl, decahydroquinolinyl, 2H,6H-1,5,2-dithiazinyl, dihydrofuro[2,3 b]tetrahydrofuran, furanyl, furazanyl, imidazolidinyl, imidazolinyl, imidazolyl, 1H-indazolyl, indolenyl, indolinyl, indolizinyl, indolyl, 3H-indolyl, isatinoyl, isobenzofuranyl, isochromanyl, isoindazolyl, isoindolinyl, isoindolyl, isoquinolinyl, isothiazolyl, isoxazolyl, methylenedioxyphenyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxindolyl, pyrimidinyl, phenanthridinyl, phenanthrolinyl, phenazinyl, phenothiazinyl, phenoxathinyl, phenoxazinyl, phthalazinyl, piperazinyl, piperidinyl, piperidonyl, 4-piperidonyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl and xanthenyl, and the like. In some embodiments, 1, 2, 3, or 4 hydrogen atoms of each ring can be substituted by a substituent.

The term “heteroaryl” refers to an aromatic 5-8 membered monocyclic, 8-12 membered fused bicyclic, or 11-14 membered fused tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively. Cₓ heteroaryl and Cₓ-C_{y}heteroaryl are typically used where X and Y indicate the number of carbon atoms in the ring system. Heteroaryls include, but are not limited to, those derived from benzo[b]furan, benzo[b] thiophene, benzimidazole, imidazo[4,5-c]pyridine, quinazoline, thieno[2,3-c]pyridine, thieno[3,2-b]pyridine, thieno[2, 3-b]pyridine, indolizine, imidazo[1,2a]pyridine, quinoline, isoquinoline, phthalazine, quinoxaline, naphthyridine, quinolizine, indole, isoindole, indazole, indoline, benzoxazole, benzopyrazole, benzothiazole, imidazo[1,5-a]pyridine, pyrazolo[1,5-a]pyridine, imidazo[1,2-a]pyrimidine, imidazo[1,2-c]pyrimidine, imidazo[1,5-a]pyrimidine, imidazo[1,5-c]pyrimidine, pyrrolo[2,3-b]pyridine, pyrrolo[2,3c]pyridine, pyrrolo[3,2-c]pyridine, pyrrolo[3,2-b]pyridine, pyrrolo[2,3-d]pyrimidine, pyrrolo[3,2-d]pyrimidine, pyrrolo [2,3-b]pyrazine, pyrazolo[1,5-a]pyridine, pyrrolo[1,2-b]pyridazine, pyrrolo[1,2-c]pyrimidine, pyrrolo[1,2-a]pyrimidine, pyrrolo[1,2-a]pyrazine, triazo[1,5-a]pyridine, pteridine, purine, carbazole, acridine, phenazine, phenothiazene, phenoxazine,1,2-dihydropyrrolo[3,2,1-hi]indole, indolizine, pyrido[1,2-a]indole, 2(1H)-pyridinone, benzimidazolyl, benzofuranyl, benzothiofuranyl, benzothiophenyl, benzoxazolyl, benzoxazolinyl, benzthiazolyl, benztriazolyl, benztetrazolyl, benzisoxazolyl, benzisothiazolyl, benzimidazolinyl, carbazolyl, 4aH-carbazolyl, carbolinyl, chromanyl, chromenyl, cinnolinyl, decahydroquinolinyl, 2H,6H-1,5,2-dithiazinyl, dihydrofuro[2,3-b]tetrahydrofuran, furanyl, furazanyl, imidazolidinyl, imidazolinyl, imidazolyl, 1H-indazolyl, indolenyl, indolinyl, indolizinyl, indolyl, 3H-indolyl, isatinoyl, isobenzofuranyl, isochromanyl, isoindazolyl, isoindolinyl, isoindolyl, isoquinolinyl, isothiazolyl, isoxazolyl, methylenedioxyphenyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxepanyl, oxetanyl, oxindolyl, pyrimidinyl, phenanthridinyl, phenanthrolinyl, phenazinyl, phenothiazinyl, phenoxathinyl, phenoxazinyl, phthalazinyl, piperazinyl, piperidinyl, piperidonyl, 4-piperidonyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydropyranyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl and xanthenyl. Some exemplary heteroaryl groups include, but are not limited to, pyridyl, furyl or furanyl, imidazolyl, benzimidazolyl, pyrimidinyl, thiophenyl or thienyl, pyridazinyl, pyrazinyl, quinolinyl, indolyl, thiazolyl, naphthyridinyl, 2-amino-4-oxo-3,4-dihydropteridin-6-yl, tetrahydroisoquinolinyl, and the like. In some embodiments, 1, 2, 3, or 4 hydrogen atoms of each ring may be substituted by a substituent.

The term “cyclyl” or “cycloalkyl” refers to saturated and partially unsaturated cyclic hydrocarbon groups having 3 to 12 carbons, for example, 3 to 8 carbons, and, for example, 3 to 6 carbons. Cₓcyclyl and Cₓ-C_{y}cylcyl are typically used where X and Y indicate the number of carbon atoms in the ring system. The cycloalkyl group additionally can be optionally substituted, e.g., with 1, 2, 3, or 4 substituents. C₃-C₁₀cyclyl includes cyclopropyl, cyclobutyl, cyclopentyl, cyclohexyl, cyclohexenyl, 2,5-cyclohexadienyl, cycloheptyl, cyclooctyl, bicyclo[2.2.2]octyl, adamantan-1-yl, decahydronaphthyl, oxocyclohexyl, dioxocyclohexyl, thiocyclohexyl, 2-oxobicyclo [2.2.1]hept-1-yl, and the like.

Aryl and heteroaryls can be optionally substituted with one or more substituents at one or more positions, for example, halogen, alkyl, aralkyl, alkenyl, alkynyl, cycloalkyl, hydroxyl, amino, nitro, sulfhydryl, imino, amido, phosphate, phosphonate, phosphinate, carbonyl, carboxyl, silyl, ether, alkylthio, sulfonyl, ketone, aldehyde, ester, a heterocyclyl, an aromatic or heteroaromatic moiety, —CF3, —CN, or the like.

The term “heterocyclyl” refers to a nonaromatic 5-8 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively). Cₓheterocyclyl and Cₓ-C_{y}heterocyclyl are typically used where X and Y indicate the number of carbon atoms in the ring system. In some embodiments, 1, 2 or 3 hydrogen atoms of each ring can be substituted by a substituent. Exemplary heterocyclyl groups include, but are not limited to piperazinyl, pyrrolidinyl, dioxanyl, morpholinyl, tetrahydrofuranyl, piperidyl, 4-morpholyl, 4-piperazinyl, pyrrolidinyl, perhydropyrrolizinyl, 1,4-diazaperhydroepinyl, 1,3-dioxanyl, 1,4-dioxanyl and the like.

The terms “bicyclic” and “tricyclic” refers to fused, bridged, or joined by a single bond polycyclic ring assemblies.

The term “cyclylalkylene” means a divalent aryl, heteroaryl, cyclyl, or heterocyclyl.

As used herein, the term “carbonyl” means the radical —C(O)—. It is noted that the carbonyl radical can be further substituted with a variety of substituents to form different carbonyl groups including acids, acid halides, amides, esters, ketones, and the like.

The term “carboxyl” refers to a functional group with the formula —COOH.

The term “carboxy” means the radical —C(O)O—. It is noted that compounds described herein containing carboxy moieties can include protected derivatives thereof, i.e., where the oxygen is substituted with a protecting group. Suitable protecting groups for carboxy moieties include benzyl, tert-butyl, and the like.

The term “cyano” means the radical —CN.

The term “isocyano,” as used herein, refers to a group of the formula —NC.

The term “thiocyano” refers to the radical —SCN.

As used herein, the term “isothiocyanato” refers to a —NCS group.

The term, “heteroatom” refers to an atom that is not a carbon atom. Particular examples of heteroatoms include, but are not limited to nitrogen, oxygen, sulfur and halogens. A “heteroatom moiety” includes a moiety where the atom by which the moiety is attached is not a carbon. Examples of heteroatom moieties include —N═, —NR^{N}—, —N⁺(O⁻)═, —O—, —S— or —S(O)₂—, —OS(O)₂—, and —SS—, wherein R^{N} is H or a further substituent.

The term “hydroxyl” means the radical —OH.

The term “nitro” means the radical —NO₂.

The term “azide” means —N₃.

As used herein, the term, “aromatic” means a moiety wherein the constituent atoms make up an unsaturated ring system, all atoms in the ring system are sp² hybridized and the total number of pi electrons is equal to 4n+2. An aromatic ring can be such that the ring atoms are only carbon atoms (e.g., aryl) or can include carbon and non-carbon atoms (e.g., heteroaryl).

As used herein, the term “substituted” refers to independent replacement of one or more (typically 1, 2, 3, 4, or 5) of the hydrogen atoms on the substituted moiety with substituents independently selected from the group of substituents listed below in the definition for “substituents” or otherwise specified. In general, a non-hydrogen substituent can be any substituent that can be bound to an atom of the given moiety that is specified to be substituted. Examples of substituents include, but are not limited to, acyl, acylamino, acyloxy, aldehyde, alicyclic, aliphatic, alkanesulfonamido, alkanesulfonyl, alkaryl, alkenyl, alkoxy, alkoxycarbonyl, alkyl, alkylamino, alkylcarbanoyl, alkylene, alkylidene, alkylthios, alkynyl, amide, amido, amino, amino, aminoalkyl, aralkyl, aralkylsulfonamido, arenesulfonamido, arenesulfonyl, aromatic, aryl, arylamino, arylcarbanoyl, aryloxy, azido, carbamoyl, carbonyl, carbonyls (including ketones, carboxy, carboxylates, CF₃, cyano (CN), cycloalkyl, cycloalkylene, ester, ether, haloalkyl, halogen, halogen, heteroaryl, heterocyclyl, hydroxy, hydroxy, hydroxyalkyl, imino, iminoketone, ketone, mercapto, nitro, oxaalkyl, oxo, oxoalkyl, phosphoryl (including phosphonate and phosphinate), silyl groups, sulfonamido, sulfonyl (including sulfate, sulfamoyl and sulfonate), thiols, and ureido moieties, each of which may optionally also be substituted or unsubstituted. In some cases, two substituents, together with the carbon(s) to which they are attached to, can form a ring.

The terms “alkoxyl” or “alkoxy” as used herein refers to an alkyl group, as defined above, having an oxygen radical attached thereto. Representative alkoxyl groups include methoxy, ethoxy, propyloxy, tert-butoxy, n-propyloxy, iso-propyloxy, n-butyloxy, iso-butyloxy, and the like. An “ether” is two hydrocarbons covalently linked by an oxygen. Accordingly, the substituent of an alkyl that renders that alkyl an ether is or resembles an alkoxyl, such as can be represented by one of —O-alkyl, —O-alkenyl, and —O-alkynyl. Aroxy can be represented by —O-aryl or O-heteroaryl, wherein aryl and heteroaryl are as defined below. The alkoxy and aroxy groups can be substituted as described above for alkyl.

The term “aralkyl”, as used herein, refers to an alkyl group substituted with an aryl group (e.g., an aromatic or heteroaromatic group).

The term “alkylthio” refers to an alkyl group, as defined above, having a sulfur radical attached thereto. In preferred embodiments, the “alkylthio” moiety is represented by one of —S-alkyl, —S-alkenyl, and —S-alkynyl. Representative alkylthio groups include methylthio, ethylthio, and the like. The term “alkylthio” also encompasses cycloalkyl groups, alkene and cycloalkene groups, and alkyne groups. “Arylthio” refers to aryl or heteroaryl groups.

The term “sulfinyl” means the radical —SO—. It is noted that the sulfinyl radical can be further substituted with a variety of substituents to form different sulfinyl groups including sulfinic acids, sulfinamides, sulfinyl esters, sulfoxides, and the like.

The term “sulfonyl” means the radical —SO₂—. It is noted that the sulfonyl radical can be further substituted with a variety of substituents to form different sulfonyl groups including sulfonic acids (—SO₃H), sulfonamides, sulfonate esters, sulfones, and the like.

The term “sulfo” means HOSO₂—.

The term “sulfino” means HO₂S—.

As used herein, thiol means —SH.

As used herein, the term “amino” means —NH₂. The term “alkylamino” means a nitrogen moiety having at least one straight or branched unsaturated aliphatic, cyclyl, or heterocyclyl radicals attached to the nitrogen. For example, representative amino groups include —NH₂, —NHCH₃, —N(CH₃)₂, —NH(C₁-C₁₀alkyl), —N(C₁-C₁₀alkyl)₂, and the like. The term “alkylamino” includes “alkenylamino,” “alkynylamino,” “cyclylamino,” and “heterocyclylamino.” The term “arylamino” means a nitrogen moiety having at least one aryl radical attached to the nitrogen. For example —NHaryl, and —N(aryl)₂. The term “heteroarylamino” means a nitrogen moiety having at least one heteroaryl radical attached to the nitrogen. For example —NHheteroaryl, and —N(heteroaryl)₂. Optionally, two substituents together with the nitrogen can also form a ring. Unless indicated otherwise, the compounds described herein containing amino moieties can include protected derivatives thereof. Suitable protecting groups for amino moieties include acetyl, tertbutoxycarbonyl, benzyloxycarbonyl, and the like.

The term “aminoalkyl” means an alkyl, alkenyl, and alkynyl as defined above, except where one or more substituted or unsubstituted nitrogen atoms (—N—) are positioned between carbon atoms of the alkyl, alkenyl, or alkynyl. For example, an (C₂-C₆) aminoalkyl refers to a chain comprising between 2 and 6 carbons and one or more nitrogen atoms positioned between the carbon atoms.

The term “alkoxycarbonyl” means —C(O)O-(alkyl), such as —C(═O)OCH₃, —C(═O)OCH₂CH₃, and the like.

The term “aryloxy” means —O-(aryl), such as —O-phenyl, —O-pyridinyl, and the like.

The term “arylalkyl” means -(alkyl)-(aryl), such as benzyl (i.e., —CH₂phenyl), —CH₂-pyrindinyl, and the like.

The term “aminoalkoxy” means —O-(alkyl)-NH₂, such as —OCH₂NH₂, —OCH₂CH₂NH₂, and the like.

The term “mono- or di-alkylamino” means —NH(alkyl) or —N(alkyl)(alkyl), respectively, such as —NHCH₃, —N(CH₃)₂, and the like.

The term “mono- or di-alkylaminoalkoxy” means —O-(alkyl)-NH(alkyl) or —O-(alkyl)-N(alkyl)(alkyl), respectively, such as —OCH₂NHCH₃, —OCH₂CH₂N(CH₃)₂, and the like.

The term “arylamino” means —NH(aryl), such as —NH-phenyl, —NH-pyridinyl, and the like.

The term “alkylamino” means —NH(alkyl), such as —NHCH₃, —NHCH₂CH₃, and the like.

It is noted in regard to all of the definitions provided herein that the definitions should be interpreted as being open ended in the sense that further substituents beyond those specified may be included. Hence, a C₁ alkyl indicates that there is one carbon atom but does not indicate what are the substituents on the carbon atom. Hence, a C₁ alkyl comprises methyl (i.e., —CH3) as well as —CRₐR_{b}R_{c} where Rₐ, R_{b}, and R_{c} can each independently be hydrogen or any other substituent where the atom alpha to the carbon is a heteroatom or cyano. Hence, CF₃, CH₂OH and CH₂CN are all C₁ alkyls.

Unless otherwise stated, structures depicted herein are meant to include compounds which differ only in the presence of one or more isotopically enriched atoms. For example, compounds having the present structure except for the replacement of a hydrogen atom by a deuterium or tritium, or the replacement of a carbon atom by a ¹³C- or ¹⁴C-enriched carbon are within the scope of the invention.

As used here in the term “isomer” refers to compounds having the same molecular formula but differing in structure. Isomers which differ only in configuration and/or conformation are referred to as “stereoisomers.”

The term “nucleophilic” refers to a functional member that is electron rich, has an unshared pair of electrons acting as a reactive site, and reacts with a positively charged or electron-deficient site, generally present on another molecule.

The term “nucleophile” refers to a compound having a nucleophilic site.

The term “pharmaceutically acceptable carrier” as used herein means a pharmaceutically acceptable material, composition or vehicle, such as a liquid or solid filler, diluent, excipient, solvent or encapsulating material, involved in maintaining the activity of or carrying or transporting the subject agents from one organ, or portion of the body, to another organ, or portion of the body. In addition to being “pharmaceutically acceptable” as that term is defined herein, each carrier must also be “acceptable” in the sense of being compatible with the other ingredients of the formulation. The pharmaceutical formulation contains a compound of the invention in combination with one or more pharmaceutically acceptable ingredients. The carrier can be in the form of a solid, semi-solid or liquid diluent, cream or a capsule. These pharmaceutical preparations are a further object of the invention. Usually the amount of active compounds is between 0.1-95% by weight of the preparation, preferably between 0.2-20% by weight in preparations for parenteral use and preferably between 1 and 50% by weight in preparations for oral administration. For the clinical use of the methods of the present invention, targeted delivery composition of the invention is formulated into pharmaceutical compositions or pharmaceutical formulations for parenteral administration, e.g., intravenous; mucosal, e.g., intranasal; enteral, e.g., oral; topical, e.g., transdermal; ocular, e.g., via corneal scarification or other mode of administration. The pharmaceutical composition contains a compound of the invention in combination with one or more pharmaceutically acceptable ingredients. The carrier can be in the form of a solid, semi-solid or liquid diluent, cream or a capsule.

The terms “subject” and “individual” are used interchangeably herein, and refer to an animal, for example a human. The term “subject” as used herein refers to human and non-human animals. The term “non-human animals” and “non-human mammals” are used interchangeably herein includes all vertebrates, e.g., mammals, such as non-human primates, (particularly higher primates), sheep, dog, rodent (e.g. mouse or rat), guinea pig, goat, pig, cat, rabbits, cows, and non-mammals such as chickens, amphibians, reptiles etc. In one embodiment, the subject is human. In another embodiment, the subject is an experimental animal or animal substitute as a disease model. The term does not denote a particular age or sex. Thus, adult and newborn subjects, as well as fetuses, whether male or female, are intended to be covered. Examples of subjects include humans, dogs, cats, cows, goats, and mice. The term subject is further intended to include transgenic species.

The singular terms “a,” “an,” and “the” include plural referents unless context clearly indicates otherwise. Similarly, the word “or” is intended to include “and” unless the context clearly indicates otherwise.

Other than in the operating examples, or where otherwise indicated, all numbers expressing quantities of ingredients or reaction conditions used herein should be understood as modified in all instances by the term “about.” The term “about” when used in connection with percentages may mean ±1% of the value being referred to. For example, about 100 means from 99 to 101.

Although methods and materials similar or equivalent to those described herein can be used in the practice or testing of this disclosure, suitable methods and materials are described below. The term “comprises” means “includes.” The abbreviation, “e.g.” is derived from the Latin exempli gratia, and is used herein to indicate a non-limiting example. Thus, the abbreviation “e.g.” is synonymous with the term “for example.”

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow. Further, to the extent not already indicated, it will be understood by those of ordinary skill in the art that any one of the various embodiments herein described and illustrated can be further modified to incorporate features shown in any of the other embodiments disclosed herein.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

### EXAMPLES

The following examples illustrate some embodiments and aspects of the invention. It will be apparent to those skilled in the relevant art that various modifications, additions, substitutions, and the like can be performed without altering the spirit or scope of the invention, and such modifications and variations are encompassed within the scope of the invention as defined in the claims which follow. The following examples do not in any way limit the invention.

The technology described herein is further illustrated by the following examples which in no way should be construed as being further limiting.

### Example 1

### Materials and Methods for a Chemical Strategy for the Cell-Based Detection of HDAC Activity

### General Materials and Methods

All chemical reagents were of ACS grade purity or higher, purchased from commercial sources, and used as received without further purification. Reactions were performed using standard techniques, including inert atmosphere of nitrogen with standard Schlenk technique, when necessary. Glassware was oven-dried at 150° C. overnight. Analytical thin layer chromatography (TLC) was performed on SiliCycle TLC silica Gel 60-F254 plates with visualization by ultraviolet (UV) irradiation at 254 nm. Purifications were performed using HP silica chromatography column by Teledyne Isco. The elution system for each purification was determined by TLC analysis. Chromatography solvents were purchased from commercial sources and used without distillation. NMR spectra were recorded at 22° C. on a Varian 500 MHz spectrometer. Proton chemical shifts are reported as δ in units of parts per million (ppm) relative to chloroform-d (δ 7.27, singlet), methanol-d4 (δ 3.31, pentet), or dimethylsulfoxide-d6 (δ 2.50, pentet). Multiplicities are reported as follows: s (singlet), d (doublet), t (triplet), q (quartet), dd (doublet of doublets), dt (doublet of triplets), dq (doublet of quartet) or m (multiplet). Coupling constants are reported as a J value in Hertz (Hz). ¹³C NMR chemical shifts are reported as 6 in units of parts per million (ppm) relative to chloroform-d (δ 77.1, triplet), methanol-d4 (δ 49.0, septet), dimethyl sulfoxide-d6 (δ 39.5 septet), or acetonitrile-d3 (δ 1.3, singlet; 118.3 septet). HPLC-analysis of organic synthetic reactions was conducted on an Agilent 1100 series HPLC fitted with a diode-array detector, quaternary pump, vacuum degasser, and autosampler. Mass spectrometry data were recorded on an Agilent 6310 ion trap mass spectrometer (ESI source) connected to an Agilent 1200 series HPLC with quaternary pump, vacuum degasser, diode-array detector, and autosampler. Analytical separation by HPLC was achieved by a gradient of acetonitrile with 0.01% ammonium formate (10% for 0-3 minutes, 10%-95% for 3-13 minutes, 95% for 13-15 minutes; percentages are % acetonitrile, v/v).

### Synthesis Procedures and Characterization Data for HP-1

The synthetic scheme for HP-1 is outlined in FIG. 3. The steps involved in the synthesis of Compound 1 are as follows: i & ii. To an oven-dried, round-bottom flask under N₂ was added 5-hexyn-1-ol (3.0 g, 30.57 mmol, 1.0 equiv.) and degassed anhydrous THF (80 mL). Bu₃SnH (8.10 mL, 30.57 mmol, 1.0 equiv.) was added via syringe, and the solution was heated to 80° C. AIBN (1.0 g, 6.11 mmol, 0.2 equiv.) was then added in one portion under a stream of N₂. After 5 minutes, the temperature was raised to 90° C., and the solution was stirred for 14 hours before cooling to room temperature. The solvent was removed under vacuum and the intermediate (pale yellow oil) was dissolved in 15 mL of anhydrous CH₂Cl₂. Separately, I₂ (9.31 g, 36.68 mmol, 1.2 equiv.) was dissolved in 20 mL of CH₂Cl₂ and added drop-wise to the vinyl tin solution until a purple color persisted. The reaction mixture was stirred for an additional 30 minutes, then saturated Na₂SO₃ (aq) was added to quench the reaction. The organic phase was extracted, washed with brine, dried over Na₂SO₄, and concentrated under vacuum. The residue was purified via flash column chromatography (Rf=0.5 in 1:1 hexanes/EtOAc) to give a mixture (trans:cis=2.6:1) of vinyl iodide 1 (4.21 g, 18.6 mmol, 61% yield) as a pale yellow oil. Trans ¹H NMR (500 MHz, CDCl₃):1.50 (m, 2H), 1.59 (m, 2H), 2.10 (dq, 2H, J=14.5, 1.1 Hz), 3.66 (m, 2H), 6.02 (br d, 1H, J=14.3 Hz), 6.51 (dt, 1H, J=14.3, 7.1 Hz). Trans ¹³C NMR (125 MHz, CDCl₃): 24.52, 31.87, 35.70, 62.53, 74.76, 146.19. Cis ¹H NMR (500 MHz, CDCl₃): 6.20 (m, 2H), 3.66 (m, 2H), 2.19 (q, 2H, J=6.9 Hz), 1.59 (m, 2H), 1.50 (m, 2H). Cis ¹³C NMR (125 MHz, CDCl₃): 24.13, 32.03, 34.34, 62.62, 82.67, 140.92.

The subsequent steps leading to the synthesis of Compound 2 are as follows: iii. To an oven-dried, round-bottom flask under N₂ was added anhydrous CH₂Cl₂ (30 mL), vinyl iodide (5.31 g, 24.5 mmol, 1.0 equiv.), and imidazole (2.88 g, 42.3 mmol, 1.8 equiv.). The solution was stirred for 5 minutes at room temperature, then chilled to 0° C. t-Bu(Cl)Ph₂Si (6.01 mL, 24.5 mmol, 1.0 equiv) was added dropwise via syringe and a white precipitate formed immediately. The reaction mixture was warmed to room temperature, then stirred for 1 hour. The solution was diluted with CH₂Cl₂ and washed with NH₄Cl (2×25 mL), followed by brine (1×25 mL). The organic phase was dried over Na₂SO₄, concentrated under vacuum, and purified via flash column chromatography (Rf=0.8 in 8:2 hexanes/EtOAc) to afford a mixture (trans:cis=1.57:1) of t-BDPS protected vinyl iodide (8.34 g, 17.95 mmol, 73% yield) as a colorless oil. Trans ¹H NMR (500 MHz, CDCl₃): 1.05 (s, 9H), 1.47 (m, 2H), 1.60 (m, 2H), 2.04 (q, 2H, J=7.3 Hz), 3.66 (m, 2H), 5.95 (d, 1H, J=14.3 Hz), 6.48 (dt, 1H, J=14.3, 7.1 Hz), 7.40 (m, 6H), 7.66 (m, 4H). Trans ¹³C NMR (500 MHz, CDCl₃): 19.21, 24.60, 26.85, 31.71, 35.68, 63.56, 74.53, 127.60, 129.55, 133.96, 135.55, 146.40. Cis ¹H NMR (500 MHz, CDCl₃): 1.05 (s, 9H), 1.47 (m, 2H), 1.85 (m, 2H), 2.14 (q, 2H, J=7.0 Hz), 3.75 (m, 2H), 6.16 (m, 2H), 7.40 (m, 6H), 7.66 (m, 4H). Cis ¹³C NMR (125 MHz, CDCl₃): 19.21, 24.26, 26.85, 31.95, 34.39, 63.56, 82.36, 127.59, 129.51,134.02,135.56, 141.33.

iv. To an oven-dried, round-bottom flask under N₂ was added CuI (108 mg, 0.56 mmol, 0.1 equiv.), acetamide (662 mg, 11.2 mmol, 2.0 equiv.), and Cs₂CO₃ (2.737 g, 8.4 mmol, 1.5 equiv.). The solids were suspended in anhydrous THF (6 mL), and N,N-dimethylethylenediamine (122 μL, 1.12 mmol, 0.2 equiv.) was added dropwise. Separately, the tBDPS-protected vinyl iodide was dissolved in anhydrous THF (3 mL) and added dropwise to the acetamide solution via syringe. The reaction vessel was flushed with N₂, sealed, and heated to 55° C. overnight. The reaction mixture was cooled to room temperature, diluted with EtOAc (80 mL), and filtered over a pad of silica gel. After thorough washing, the combined organic solvent was concentrated under vacuum and the mixture (trans:cis=4.6:1) was purified by flash column chromatography (cis Rf=0.45, trans Rf=0.40 in 1:1 hexanes/EtOAc). The major trans isomer of the tBDPS-protected enamide (2, 1.60 g, 4.02 mmol, 72% yield) was obtained as a colorless oil. Trans ¹H NMR (500 MHz, CDCl₃): 1.04 (s, 9H), 1.43 (m, 2H), 1.55 (m, 2H), 1.99 (m, 2H), 2.02 (s, 3H), 3.65 (t, 2H, J=6.2 Hz), 5.07 Hz (dt, 1H, J=14.2, 7.1 Hz), 6.71 (dd, 1H, J=14.2, 10.2 Hz), 6.85 (br d, 1H, J=10.2 Hz), 7.39 (m, 6H), 7.66 (dd, 4H, J=7.7, 1.3 Hz). Trans ¹³C NMR (500 MHz, CDCl₃): 19.17, 23.11, 26.08, 26.83, 26.83, 26.83, 29.37, 63.64, 112.71, 122.20, 127.53, 129.46, 134.00, 135.45, 167.10.

The subsequent steps leading to the synthesis of Compound 3 are as follows: v. To an oven-dried, round-bottom flask under N₂ was added the trans tBDPS-protected enamide (2.26 g, 5.68 mmol, 1.0 equiv.) and THF (12 mL). TBAF (8.52 mL, 1.0 M in THF, 1.5 equiv.) was added dropwise via syringe under N₂, and the reaction was stirred at room temperature overnight. The solution was then filtered over a pad of silica gel that was pre-washed with 94:5:1 CH₂Cl₂/MeOH/Et₃N. After washing thoroughly, the combined organic solvent was concentrated under vacuum and purified by flash column chromatography (Rf=0.25 in 94:5:1 CH₂Cl₂/MeOH/Et₃N). The trans hydroxy enamide (0.72 g, 4.55 mmol, 80% yield) was obtained as a white solid. Trans ¹H NMR (500 MHz, CDCl₃): 1.20 (br t, 1H, J=5.1Hz), 1.37 (m, 2H), 1.50 (m, 2H), 1.51 (s, 3H), 1.95 (s, 3H), 1.99 (m, 2H), 3.58 (dt, 2H, J=6.2, 5.1 Hz), 5.04 (dt, 1H, J=14.3, 7.1 Hz), 6.68 (dd, 1H, J=14.3, 10.6 Hz), 6.85 (br s, 1H). Trans ¹³C NMR (500 MHz, CDCl₃): 23.10, 25.93, 29.36, 32.01, 62.56, 112.67, 122.73, 167.37.

vi. To an oven-dried, round-bottom flask under N₂ was added the trans hydroxy enamide (71 mg, 0.45 mmol, 1.0 equiv.) and anhydrous CH₂Cl₂ (5 mL). The solution was chilled to 0° C., and TosCl (260 mg, 1.36 mmol, 3.0 equiv.) and anhydrous pyridine (0.22 mL, 2.72 mmol, 6.0 equiv.) were added under N₂. The reaction mixture was warmed to room temperature, and stirred for 1 hour. The solvent was removed under vacuum, and the resulting yellow oil was taken up in 5 mL of CH₂Cl₂ and re-concentrated twice. The residue was purified by flash column chromatography (Rf=0.45 in hexanes) to obtain the trans Tos-protected enamide (87 mg, 0.28 mmol, 64% yield) as a white solid. Trans ¹H NMR (500 MHz, CDCl₃): 1.39 (p, 2H, J=7.6 Hz), 1.64 (m, 2H), 1.97 (dt, 2H, J=10.9, 7.4 Hz), 2.01 (s, 3H), 2.45 (s, 3H), 4.01 (t, 2H, J=6.3 Hz), 5.04 (dt, 1H, J=14.2, 7.4 Hz), 6.68 (dd, 1H, J=14.2, 10.2 Hz), 6.97 (br d, 1H, J=10.2 Hz), 7.35 (d, 2H, J=8.1 Hz), 7.78 (d, 2H, J=8.2 Hz). Trans ¹³C NMR (125 MHz, CDCl₃): 21.48, 22.88, 25.42, 27.99, 28.84, 70.38, 111.67, 123.03, 127.65, 132.72, 144.77, 167.47.

vii. To an oven-dried, round-bottom flask under N₂ was added the trans Tos-protected enamide (883 mg, 2.83 mmol, 1.0 equiv.) and DMF (10 mL). NaN₃ (386 mg, 5.67 mmol, 2.0 equiv.) was added under a stream of N₂, and the reaction vessel was sealed and heated to 80° C. for 2 hours. The solution was cooled to room temperature and concentrated under vacuum. The resulting residue was taken up in 10 mL of CH₂Cl₂ and re-concentrated twice. The residue was then diluted in 10 mL of CH₂Cl₂ and washed with brine (3×50 mL), dried over Na₂SO₄, and concentrated under vacuum overnight to afford the trans azido enamide (3, 420 mg, 2.30 mmol, 81% yield) as a pale yellow oil. Trans ¹1-1 NMR (500 MHz, CDCl₃): 1.45 (m, 2H), 1.60 (m,2H), 2.02 (s, 3H), 2.04 (m, 2H), 3.26 (t, 2H, J=7.6 Hz), 5.09 (dt, 1H, J=14.2, 7.0 Hz), 6.75 (dd, 1H, J=14.2, 10.6 Hz), 6.99 (br s, 1H). Trans ¹³C NMR (125 MHz, CDCl₃): 23.09, 26.85, 28.14, 29.11, 51.21, 111.93, 122.99, 167.24.

The subsequent steps leading to the synthesis of Compound 4 are as follows: viii. Zinc powder (8.11 mg, 0.12 mmol, 1.3 equiv.) was added to a solution of compound 3 (17.0 mg, 0.09 mmol, 1.0 equiv.) and ammonium chloride (11.6 mg, 0.22 mmol, 2.4 equiv.) in ethyl alcohol (248 μL) and water (84 μL), and the mixture was stirred vigorously at room temperature. After completion of the reaction (2 hours, monitored by TLC), ethyl acetate (200 μL) and aqueous ammonia (10 μL) were added. The mixture was filtered, and the filtrate was washed with brine and dried over anhydrous Na₂SO₄. After removal of solvent under reduced pressure, the residue was purified by recrystallization with methylene chloride to give the corresponding amine, 4 (a mixture of trans and cis isomers in 1.5:1 ratio). Trans ¹H NMR (500 MHz, CDCl₃): 1.28 (m, 2H), 1.45 (m, 2H), 1.99 (s, 3H), 2.02 (m, 2H), 2.65 (t, 2H, J=6.7 Hz), 5.08 (dt, 1H, J=14.3, 7.1Hz), 6.70 (m, 1H), 7.11 (br s, 1H). Trans ¹³C NMR (125 MHz, CDCl₃): 23.1, 26.7, 28.14, 29.0, 50.2, 110.0, 120.7, 167.24. Cis ¹H NMR (500 MHz, CDCl₃): 1.28 (m, 2H), 1.45 (m, 2H), 2.03 (s, 3H), 2.05 (m, 2H), 2.73 (t, 2H, J=6.2 Hz), 4.72 (dt, 1H, J=16.2, 7.8 Hz), 6.70 (m, 1H), 7.65 (br s, 1H). Trans ¹³C NMR (125 MHz, CDCl₃): 23.6, 25.8, 27.1, 31.0, 51.6, 112.0, 123.7, 166.2.

The subsequent steps leading to the synthesis of Compound 5 (i.e., HP-1) are as follows: ix. NBD-SE (Succinimidyl 6-(N-(7-nitrobenz-2-oxa-1,3-diazol-4-yl)amino)hexanoate) (25.0 mg, 0.064 mmol, 1.0 equiv.) and compound 4 (10 mg, 0.064 mmol, 1.0 equiv) were mixed with triethyl amine (28.0 μL, 0.2 mmol, 3.1 equiv.) in methylene chloride (3.0 mL). The mixture was stirred at room temperature overnight. Solvents were removed under vacuum, and the crude mixture was separated by flash column chromatography to obtain compound 5 (20.74 mg, 0.048 mmol, 75%) as a bright orange solid. Trans ¹H NMR (500 MHz, CDCl₃): 1.29 (m, 2H), 1.30 (m, 2H), 1.40 (m, 2H), 1.50 (m, 2H), 1.60 (m, 2H), 1.87 (s, CH₃), 2.10 (m, 2H), 2.22 (m, 2H), 3.00 (m, 2H), 3.4 (m, 2H), 5.10 (dt, 1H, J=14.3, 7.1Hz), 6.80 (m, 1H), 7.10 (br s, 1H), 7.13 (m, 1H), 8.10 (m, 1H). Cis ¹H NMR (500 MHz, CDCl₃): 1.29 (m, 2H), 1.29 (m, 2H), 1.39 (m, 2H), 1.52 (m, 2H), 1.63 (m, 2H), 1.87 (s, CH₃), 2.13 (m, 2H), 2.20 (m, 2H), 3.02 (m, 2H), 3.4 (m, 2H), 4.98 (dt, 1H, J=16.2, 7.8Hz), 7.05 (m, 1H), 7.11 (br s, 1H), 7.12 (m, 1H), 8.12 (m, 1H). ¹³C NMR (125 MHz, CDCl₃): 23.5, 24.1, 25.0, 25.3, 27.0, 31.3, 33.4, 36.7, 38.8, 50.3, 97.8, 110.5, 122.2, 126.3, 132.1, 137.4, 139.6, 144.1, 167.7, 171.1. LCMS (m/z): 433.2 (M+H)⁺

### Synthesis Procedures and Characterization Data for HP-2

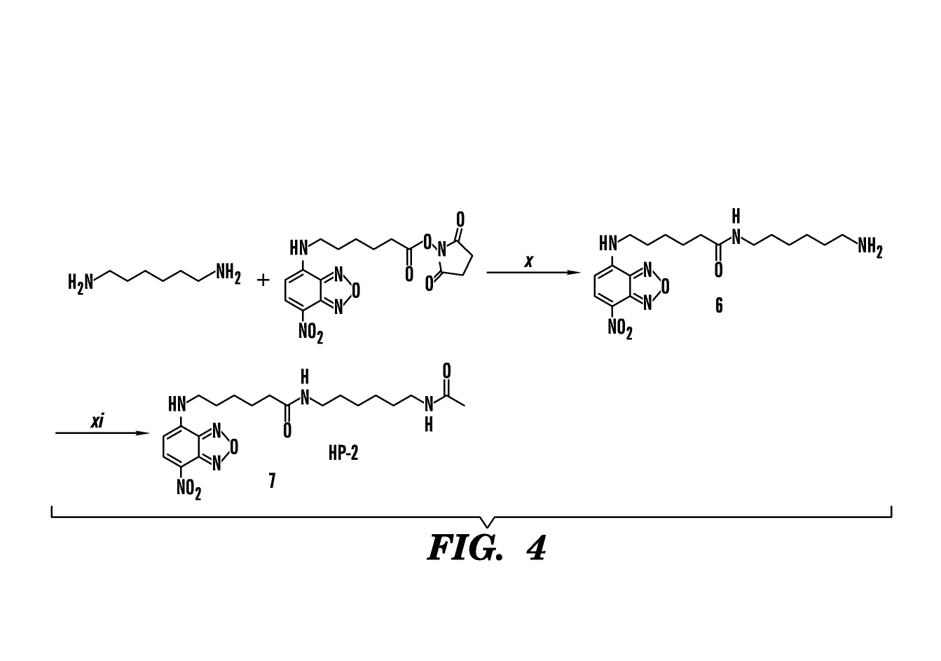

The synthetic scheme for HP-2 is outlined in FIG. 4. The steps involved in the synthesis of Compound 6 are as follows: x. Hexamethylenediamine (58.1 mg, 0.6 mmol, 1.0 equiv.) was dissolved in CH₂Cl₂ (2 mL) and cooled to 0° C. NBD-SE (25.0 mg, 0.06 mmol, 1.0 equiv.) was then dissolved in the hexamethylenediamine mixture, and the mixture was stirred at room temperature for 2 hours. Solvents were removed, and the crude mixture was purified by flash column chromatography to obtain compound 6 (15.29 mg, 0.039 mmol, 65%). ¹H NMR (500 MHz, CDCl₃): 1.29 (m, 2H), 1.29 (m, 2H), 1.30 (m, 2H), 1.30 (m, 2H), 1.51 (m, 2H), 1.52 (m, 2H), 1.60 (m, 2H), 2.11 (m, 2H), 2.23 (m, 2H), 3.00 (m, 2H), 3.4 (m, 2H), 7.13 (m, 1H), 8.10 (m, 1H). ¹³C NMR (125 MHz, CDCl₃): 24.5, 26.1, 26.4, 29.1, 31.5, 32.7, 35.5, 43.0, 50.2, 97.8, 123.4, 132.4, 136.4, 139.5, 146.0, 172.5. LCMS (m/z): 393.2 (M+H)⁺.

The subsequent steps leading to the synthesis of Compound 7 are as follows: xi. A mixture of compound 6 (30.0 mg, 0.08 mmol, 1.0 equiv.) in CH₂Cl₂ was made basic by adding 1 drop of 1 M NaOH. Acetic anhydride (72 μL, 0.80 mmol, 10 equiv.) was added, and the mixture was stirred at room temperature overnight. Solvents were removed and the residue was separated by flash column chromatography to obtain compound 7 (27.78 mg, 0.064 mmol, 80%) as a bright orange solid. ¹H NMR (500 MHz, CDCl₃): 1.29 (m, 2H), 1.29 (m, 2H), 1.30 (m, 2H), 1.30 (m, 2H), 1.51 (m, 2H), 1.52 (m, 2H), 1.60 (m, 2H), 1.80 (s, 3H), 2.11 (m, 2H), 2.23 (m, 2H), 3.00 (m, 2H), 3.4 (m, 2H), 7.13 (m, 1H), 8.10 (m, 1H). ¹³C NMR (125 MHz, CDCl₃): 23.0, 24.5, 26.1, 26.4, 29.1, 31.5, 32.7, 35.5, 43.0, 50.2, 97.8, 123.4, 132.4, 136.4, 139.5, 146.0, 170.0, 172.5. LCMS (m/z): 435.2 (M+H)⁺.

### Synthesis of Deacetylated HP-1 (DHP-1, Compound 8)

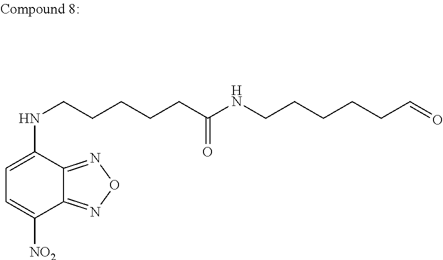 [SMILES: O=CCCCCCNC(=O)CCCCCNc1ccc([N+](=O)[O-])c2nonc12]

Compound 5 (20.0 mg, 0.05 mmol) was dissolved in 2 mL of methanol and treated with 1M HCl (5 μL) and stirred at room temperature for 1 hour. The mixture was neutralized with 1 M NaOH, and the solvents were removed under vacuum. The obtained crude product was purified by column chromatography to obtain compound 8 (18.57 mg, 0.045 mmol, 95%) as a bright yellow solid. ¹H NMR (500 MHz, CDCl₃): 1.29 (m, 2H), 1.30 (m, 2H), 1.40 (m, 2H), 1.50 (m, 2H), 1.60 (m, 2H), 1.87 (s, CH₃), 2.10 (m, 2H), 2.22 (m, 2H), 3.00 (m, 2H), 3.4 (m, 2H), 7.10 (br s, 1H), 7.13 (m, 1H), 8.10 (m, 1H), 9. 71 (s, 1H). ¹³C NMR (125 MHz, CDCl₃): 23.5, 24.1, 25.0, 25.3, 27.0, 31.3, 33.4, 36.7, 38.8, 45.3, 97.8, 110.5, 122.2, 126.3, 132.1, 137.4, 139.6, 144.1, 167.7, 171.1, 201.1. LCMS (m/z): 392.2 (M+H)⁺.

### Synthesis of Non-Fluorescent Benzoyl Analogue of HP-1 (Compound 9)

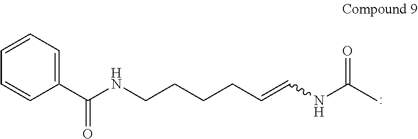 [SMILES: CC(=O)NC=CCCCCNC(=O)c1ccccc1]

Benzoyl chloride (15.0 μL, 0.128 mmol, 1.0 equiv.) and compound 4 (20 mg, 0.128 mmol, 1.0 equiv.) were mixed with triethyl amine (14.0 μL, 0.1 mmol, 0.78 equiv.) in methylene chloride (3.0 mL). The mixture was stirred at room temperature overnight. Solvents were removed under vacuum and the resultant crude mixture was separated by flash column chromatography to obtain compound 9 (21.33 mg, 0.082, 64%) as a white solid. ¹H NMR (500 MHz, CDCl₃): 1.38 (m, 2H), 1.63 (m, 2H), 1.91 (s, 3H), 2.13 (m, 2H), 3.45 (m, 2H), 5.10 (dt, 1H, J=15, 7.5Hz), 7.01 (m, 1H), 7.45 (m, 2H), 7.56 (m, 1H), 7.75 (m, 2H). ¹³C NMR (125 MHz, CDCl₃): 23.4, 24.5, 26.7, 31.0, 38.9, 110.5, 126, 127.2, 128.3, 129.0, 130.1, 132.4, 168.0, 170.1. LCMS (m/z): 261.2 (M+H)⁺.

### HDAC Buffer Preparation for Biological Assays

100 mL 1M KCl, 50 mL 1M HEPES, pH 7.4 (Gibco, 15630-114), 5 mL 10% BSA (Invitrogen, P2489), and 20 uL 50% Tween-20 (Zymed, 00-3005) were added to Milli-Q water to a final volume of 1 L, and the pH was adjusted to 7.4 (final concentrations: HEPES=50 mM, KCl=100 mM, BSA=0.05%, and Tween-20=0.001%). The buffer was stored in 45 mL aliquots at −80° C.

### Evaluation of the Stability of the Enamide (Compound 9)

HDAC buffer was adjusted to pH 2, 4, 6, 7, 8, 10 or 12 with 1 M HCl. Compound 9 was dissolved in HDAC buffer at each pH to obtain 200 μL of a 20 μM solution. The solutions were kept at room temperature and 10 μL aliquots were analyzed at selected time points (t=0, 5, 10, 15, 30 and 60 minutes). Experiments were carried out in duplicate and the hydrolysis was monitored by HPLC and LCMS. HPLC measurements were made using a gradient elution system composed of water and acetonitrile with 0.01% ammonium formate at a flow rate of 1 mL/min on an Agilent Eclipse XDB-C18 chromatographic column (4.6 mm×150 mm). LCMS was performed with a gradient elution system composed of water and acetonitrile with 0.1% ammonium formate at a flow rate of 0.1 mL/min using an Agilent Eclipse XDB-C8 chromatographic column (5 μm particle size, 2.1 mm×150 mm).

### LCMS Characterization of HDAC Enzymatic Action on HP-1 and HP-2

The enzymatic cleavage of HP-1 and HP-2 was analyzed by performing LCMS assays with HeLa nuclear extract (AnaSpec), HeLa whole cell lysate (Santa Cruz Inc.), and the following purified HDAC isoforms: 1, 2, 3, 6 and 8 (HDAC1, 3 and 8 from Cayman Chemicals and HDAC2 and 6 provided by Dr. Stephen Haggarty). Solutions containing (i) vehicle with DMSO in HDAC buffer, (ii) 5 μM probe in HDAC buffer, (iii) 5 μM probe and HDAC enzyme/HeLa nuclear extract/HeLa whole cell lysate in HDAC buffer, or (iv) 5 μM probe and HDAC enzyme/HeLa nuclear extract/HeLa whole cell lysate pre-incubated with 10 μM SAHA (Provided by Dr. Stephen Haggarty) in HDAC buffer were incubated for 12 hours at 37° C. Following incubation, a 20 μL aliquot of supernatant from each sample was analyzed by LCMS. Deacetylation of HP-1 and 2 was confirmed by detection of the (M+H)⁺.ion following positive electrospray ionization. For HP-1, the (M+H)⁺ ion for the corresponding aldehyde (8, DHP-1) was detected, which formed following reaction of the initial imine deacetylation product with water. For HP-2, the (M+H)⁻. ion for the corresponding amine (6, DHP-2) was identified. The peak area for each detected compound was measured to determine the % deacetylation of each probe. All assay samples were analyzed in triplicate. LCMS measurements were performed with a gradient elution system (5-95%) composed of water and acetonitrile with 0.1% ammonium formate at a flow rate of 0.1 mL/min using an Agilent Eclipse XDB-C8 chromatographic column (5 μm particle size, 2.1 mm×150 mm).

LCMS Analysis of HP-1 Cleavage by HDAC3 Enzyme Over Time and Determination of the Observed Rate Constant (k_{obs}) and the Half Life (T_{1/2})

The rate of cleavage of HP-1 by purified HDAC3 was analyzed by performing a LCMS assay. Solutions containing 5 μM HP-1 and HDAC3 in HDAC buffer were incubated for 12 hours at 37° C. Following incubation, a 20 μL aliquot of supernatant from each sample was analyzed by LCMS at t=0, 1, 2, 4, 8, and 12 hours. Deacetylation of HP-1 was confirmed by detection of the (M+H)⁺ ion following positive electrospray ionization. For HP-1, the (M+H)⁺ ion for the corresponding aldehyde (8, DHP-1) was detected, which formed following reaction of the initial imine deacetylation product with water. The peak area for each detected compound was measured to determine the % deacetylation of each probe. The observed rate constant was determined using Graphpad by plotting Ln(DHP-1 peak area) versus time. Assuming that this is a first order reaction, T_{1/2} (where, T_{1/2}=ln 2/k_{obs}) was estimated. All assay samples were analyzed in triplicate. LCMS measurements were performed with a gradient elution system (5-95%) composed of water and acetonitrile with 0.1% ammonium formate at a flow rate of 0.1 mL/min using an Agilent Eclipse XDB-C8 chromatographic column (5 μm particle size, 2.1 mm×150 mm).

### Characterization of Sirtuin (HDAC Class III) and Protease Enzymatic Action on HP-1

The enzymatic cleavage of HP-1 was analyzed by performing LCMS assays with sirtuin 1 and 3 (Cayman Chemicals) and the proteases chymotrypsin (Sigma-Aldrich), cathepsin (EMD Millipore), and pepsin* (Sigma-Aldrich). Solutions containing (i) vehicle with DMSO in HDAC buffer, (ii) 5 μM HP-1 in HDAC buffer, and (iii) 5 μM HP-1 and the enzyme in HDAC buffer were incubated for 12 hours at 37° C. Following incubation, a 20 μL aliquot of supernatant from each sample was analyzed by LCMS. Deacetylation of HP-1 was investigated by screening for the DHP-1 (M+H)⁺ ion following positive electrospray ionization. All assay samples were analyzed in triplicate. LCMS measurements were performed with a gradient elution system (5-95%) composed of water and acetonitrile with 0.1% ammonium formate at a flow rate of 0.1 mL/min using an Agilent Eclipse XDB-C8 chromatographic column (5 μm particle size, 2.1 mm×150 mm).

*Analysis for pepsin enzyme with HP-1 was carried out in HDAC buffer at pH 4.

### Results

HP-1 was not altered by sirtuins 1 and 3 or the proteases chymotrypsin, cathepsin and pepsin, as determined by LCMS. Positive controls were completed to verify that sirtuin 1 and 3, chymotrypsin, pepsin, and cathepsin B were active in the conditions used for the HP-1 cleavage assays. For sirtuins 1 and 3, the SIRTainty kit (EMD Millipore, Billerica, Mass.) was used, and the Pierce Protease Assay kit (Thermo Scientific, Rockford, Ill.) was used for chymotrypsin. For Cathepsin B, the cleavage of the enzyme substrate, z-RR-pNA (Santa Cruz Biotechnology, Dallas, Tex.) was monitored using a Wallac EnVision 2103 Multilabel fluorescence plate reader (PerkinElmer, Waltham, Mass.) with a 405 nm excitation filter, a 406 nm emission filter, and a gain of 150. The cleavage of the pepsin substrate, Ac-Phe-Tyr-OH (Chem-Impex Inc.), was monitored by LCMS at pH 4.

### IC₅₀ Measurements

HP-1 and HP-2 IC₅₀ values for HDAC1 were determined using the Trypsin-coupled assay as well as the Caliper endpoint assay. HP-1 and HP-2 IC₅₀ values for HDAC2, HDAC3, HDAC6 and HDAC8 were determined with the Caliper endpoint assay.

### Trypsin-Coupled HDAC1 Inhibition Assay

In vitro HDAC 1 end point enzymatic assays were performed in optimized 96-well format as previously described^{3,4} with the following modifications. Reactions were performed in volume of 120 uL with 30 ng of full-length, recombinant HDAC1 (BPS Biosciences). TCEP was excluded from the assay buffer. HDAC1 was pre-incubated with varying concentrations of HP-1 or HP-2, or DMSO vehicle for 30 minutes. Fluorophore-conjugated acetyl-lysine tripeptide substrate was added at a concentration equivalent to the substrate Kₘ, 11 uM, and the deacetylation reaction was allowed to run for 45 minutes at RT. Reactions were terminated by 10 μM pan-HDAC inhibitor LBH-589 (Panobinostat) and 150 nM Trypsin (Worthington Biochemical). Fluorescence intensity of the aminomethylcoumarin liberated by deacetylase and trypsin enzymatic activity was monitored at 460 nm using a multilabel plate reader (EnVision, Perkin-Elmer) every 5 minutes until stable, about 25 minutes. Dose response curves were fitted from this end point signal in GraphPad Prism 5 (GraphPad Software Inc.) The background fluorescence intensity of HP-1 or HP-2 was found to be negligible relative to that of aminomethylcoumarin.

### Caliper Microfluidic Endpoint IC₅₀ Assay

The Caliper microfluidic assay was performed at the Broad Institute exactly as previously described.⁵

### HP-1 Deacetylation and Protein Binding Assay

Solutions containing HP-1 (20 μM) in 30 μL HDAC buffer with 5% DMSO were incubated at 37° C. for four hours in the presence or absence of HDAC3 (3.6 μM). After incubation with HDAC3, NaCNBH₃ (1.4 mM) or vehicle (H₂O) and BSA (6 mg/mL) or vehicle (HDAC buffer), were added to the solutions prior to an additional incubation at 37° C. for two hours (all reactions were run in triplicate). Following the second incubation, the final solutions (50 μL) were added to G-25 columns (GE Healthcare, Buckinghamshire, UK) pre-equilibrated with milliQ water. The columns were then centrifuged at 700×g for 60 seconds before addition of milliQ water (50 μL). The columns were again centrifuged at 700×g for 60 seconds, and the eluent was combined with the eluent from the first spin to make fraction 1. Following this, 11 samples of water (100 μL each) were added to the columns, the columns were centrifuged as above, and the eluent was collected separately to obtain fractions 2-12. 75 μL of each fraction was transferred to a well in a 96-well, black, clear-bottom plate (Corning Incorporated, Corning, N.Y.) and the fluorescence was detected using an IVIS Spectrum (Caliper, Hopkinton, Mass.). To obtain the fluorescence signals, the 465 nm excitation filter, 530 nm emission filter, and a 5 second exposure were used. For analysis, the total photon flux over the area of each well was determined.

Imaging HDAC Activity in HeLa Cells with HP-1

HeLa cell culture and treatment with HP-1 and HP-2. HeLa cells (ATCC) were grown as a monolayer in Eagles Minimum Essential Medium (EMEM, GIBCO, BRL, Gaithersburg, Md.) with 10% Fetal Bovine Serum (FBS, GIBCO, BRL, Gaithersburg, Md.) and 1% penicillin/streptomycin (100 mg/mL). All cell culture dishes were maintained in a humidified atmosphere with 5% CO₂ at 37° C.

Determination of HDAC activity in HeLa cells by LC-MS. HDAC activity in HeLa cells was analyzed by performing an LCMS assay. Solutions of HP-1 and HP-2 (200 mM each) were prepared in DMSO and diluted in HDAC buffer to a final concentration of 100 μM. SAHA solutions (100 mM) were prepared in DMSO and diluted in HDAC buffer to make a 100 μM solution. All solutions were prepared immediately prior to application to the cells. HeLa cells plated in 600 mL cell culture flasks, were treated with HP-1 or 2 with ±SAHA so that the final concentrations of HP-1 and HP-2 were 5 μM in HDAC buffer (with 0.01% DMSO)±10 μM SAHA. Cells were then incubated at 37° C. for t=1, 2, 4, 8, 12 and 24 h. Following incubation, the medium was removed and the cells were washed three times with DPBS buffer. Cells were scraped off of the flask, lysed in milipore water (90 μL) using a mechanical homogenizer, centrifuged (at 14,000×g for 2 min) and 20 μL aliquot of supernatant from each sample was analyzed by LCMS. Cleavage and accumulation was confirmed by detection of the (M+H)' ion following positive electrospray ionization. The peak area for each detected compound was measured to determine the % conversion. For HP-1, the corresponding (M+H)⁺ ion was detected. For HP-2, the (M+H)⁺ ions for HP-2 and its corresponding amine (6, DHP-2) were identified. The peak area for each detected compound was measured to determine the % conversion of each probe. LCMS measurements were performed with a gradient elution system (5-95%) composed of water and acetonitrile with 0.1% ammonium formate at a flow rate of 0.1 mL/min using an Agilent Eclipse XDB-C8 chromatographic column (5 μm particle size, 2.1 mm×150 mm).

Determination of the fraction of protein-bound probe in HeLa cells by fluorescence. HDAC activity in HeLa cells was analyzed by performing IVIS analysis. Solutions of HP-1 and HP-2 (200 mM each) were prepared in DMSO and diluted in HDAC buffer to a final concentration of 100 μM. SAHA solutions (100 mM) were prepared in DMSO and diluted in HDAC buffer to make a 100 μM solution. All solutions were prepared immediately prior to application to the cells. HeLa cells plated in 600 mL cell culture flasks were treated with HP-1 with±SAHA so that the final concentration of HP-1 was 5 μM in HDAC buffer (with 0.01% DMSO)±10 μM SAHA. The cells were incubated at 37° C. for 24 h. Following incubation, the medium was removed and the cells were washed three times with DPBS buffer. Cells were scraped off of the flask and lysed in milipore water (90 μL) using a mechanical homogenizer. 90 μL from each lysed sample was added to a Micron centrifugal 30K filter device (Millipore Ireland Ltd. Tullagree Carrigtwohill Co. Cork, Ireland). Each device was centrifuged at 14000×g for 10 minutes. 75 μL aliquots of each concentrate fraction and corresponding original sample (before concentrating) were transferred to a 96-well, black, clear-bottom plate (Corning Incorporated, Corning, N.Y.) and the fluorescence was detected using an IVIS Spectrum (Caliper, Hopkinton, Mass.). To obtain the fluorescence signals, the 465 nm excitation filter, 530 nm emission filter, and a 1 second exposure were used. For analysis, the total photon flux over the area of each well was determined.

Imaging HDAC activity in HeLa cells with HP-1 and 2. An acid-washed, poly-lysine-treated sterile glass cover slip was added to each well of 6-well plate, and HeLa cells were plated at a seeding density of ˜2.5×10⁵ cells/mL in 2 mL of growth medium. After 24 hours, the cells reached 80-85% confluence. For all experiments, solutions of HP-1 and HP-2 (200 mM each) were prepared in DMSO and diluted in HDAC buffer to a final concentration of 100 μM. SAHA solutions (100 mM) were prepared in DMSO and diluted in HDAC buffer to make a 100 μM solution. All solutions were prepared immediately prior to application to the cells. Cells were washed with HDAC buffer and treated with HP-1, HP-1 with SAHA, HP-2 or HP-2 with SAHA so that the final concentrations of HP-1 and HP-2 were 5 μM in HDAC buffer (with 0.01% DMSO). Cells were treated with SAHA (10 μM in HDAC buffer with 0.01% DMSO) or vehicle (HDAC buffer with 0.01% DMS0) 15 min prior to treatment with HP-1 or HP-2. After HP-1 or HP-2 was added, the cells were incubated at 37° C. for 2 hours. Following incubation, the medium was removed and the cells were washed three times with 2 mL HDAC buffer per well. The buffer was removed and 2 mL 4% paraformaldehyde in PBS was added to each well and incubated for 20 minutes at 4° C. to fix the cells. The fixative was removed and cells were gently washed twice with 2 mL DPBS and twice with 2 mL deionized water. A drop of Gel Mount (anti-fade with DAPI nuclear stain) was added to microscope slides, and the cover glasses containing HeLa cells were carefully transferred to the microscope slides. After the slides dried overnight in a dark drawer, they were imaged as described below.

Confocal fluorescence image acquisition and analysis. Confocal fluorescence imaging was performed with a Zeiss laser scanning microscope 710 with a 63× objective lens and Zen 2009 software (Carl Zeiss). HP-1 and HP-2 were excited using a 488 nm Ar laser, and emission was collected using a META detector between 500 and 650 nm. DAPI was excited with a 405 nm diode laser, and emission was collected using a META detector between 450 and 500 nm. One representative image from each coverslip was collected. Each experimental condition was run in triplicate in each of three independent experiments, for a total n of 9 per treatment. The mean fluorescence intensity of 10 cells per coverslip was measured using ImageJ software. Cells were defined using a free-form selection tool using the brightfield image as a guide. The mean background signal was also measured and subtracted from the mean fluorescence signal within the cells. Mean fluorescence intensities and standard deviations were plotted in Microsoft Excel.

References for Example 1:

1. Huang, X.; Shao, N.; Palani, A.; Aslanian, R., Tetrahedron letters 2007, 48 (11), 1967-1971.

2. Jiang, L.; Job, G. E.; Klapars, A.; Buchwald, S. L., Copper-catalyzed coupling of amides and carbamates with vinyl halides. Organic letters 2003, 5 (20), 3667-3669.

3. Bradner, J. E.; West, N.; Grachan, M. L.; Greenberg, E. F.; Haggarty, S. J.; Warnow, T.; Mazitschek, R., Chemical phylogenetics of histone deacetylases. Nature chemical biology 2010, 6 (3), 238-243.

4. Fass, D. M.; Shah, R.; Ghosh, B.; Hennig, K.; Norton, S.; Zhao, W.-N.; Reis, S. A.; Klein, P. S.; Mazitschek, R.; Maglathlin, R. L., Short-chain HDAC inhibitors differentially affect vertebrate development and neuronal chromatin. ACS Medicinal Chemistry Letters 2010, 2 (1), 39-42.

5. Wagner, F. F.; Olson, D. E.; Gale, J. P.; Kaya, T.; Weïwer, M.; Aidoud, N.; Thomas, M.; Davoine, E. L.; Lemercier, B. C.; Zhang, Y.-L., Potent and selective inhibition of Histone Deacetylase 6 (HDAC6) does not require a surface-binding motif. Journal of medicinal chemistry 2013.

### Example 2

### A Chemical Strategy for the Cell-Based Detection of HDAC Activity

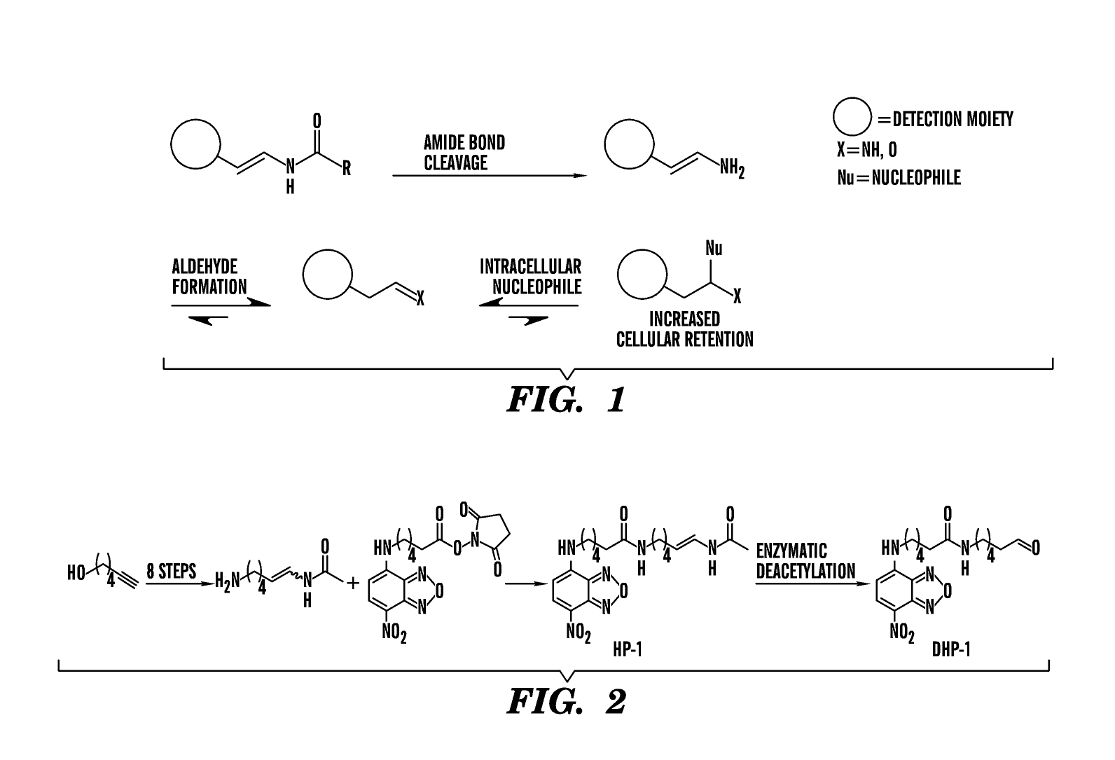

The inventors have demonstrated support for the enamide strategy through reaction with and detection of the activity of a specific class of enzymes, the histone deacetylases (HDACs).¹⁶ HDACs regulate the level of 8-amino acetylation of histone lysine residues, thereby controlling transcriptional regulation via chromatin remodeling.¹⁶⁻²⁰ Published reports indicate that irregular transcription resulting from altered expression levels of HDACs is associated with cancer, neurodegenerative diseases, and psychiatric conditions, making HDACs important drug targets for these diseases.²¹⁻³⁰ To detect HDAC deacetylation, an enamide bearing an N-acetyl group was used, which forms an aldehyde following deacetylation, thus leading to intracellular accumulation (FIG. 1). Existing HDAC activity-based probes require UV light-induced photocrosslinking for enzyme localization, which is incompatible for in vivo studies.^{31,32} A fluorescent probe, HDAC Probe-1 (HP-1), was designed for proof of concept studies aimed at demonstrating HDAC-specific intracellular accumulation. HP-1 is a derivative of 7-nitrobenzo-2-oxa-1,3-diazole (NBD) that bears an aliphatic linker, akin to a lysine side-chain, with a terminal enamide (FIGS. 2 and 3). During the synthesis of HP-1, it was found that the trans and cis isomers were non-isolable, with the isomers consistently obtained in a 1.5:1 trans:cis mixture. Therefore, all experiments were completed using this isomeric mixture. In addition to HP-1, HDAC probe-2 (HP-2, FIG. 4) was synthesized, which lacks the double bond present in HP-1 and therefore cannot tautomerize to an aldehyde. Comparison of HP-1 and 2 was used to determine the impact of the double bond on deacetylation selectivity among HDAC isoforms and the necessity of aldehyde formation for intracellular retention of deacetylated HP-1 (DHP-1).

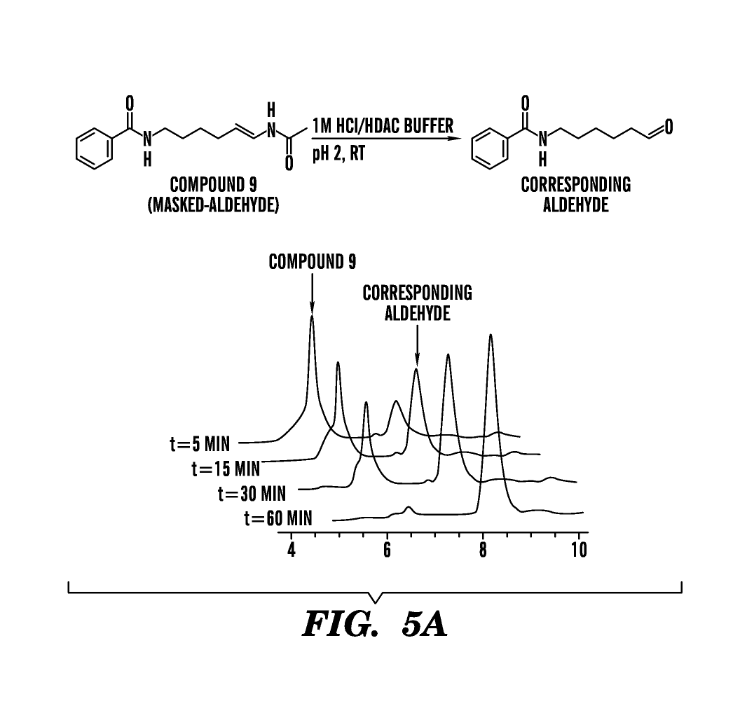

The first studies involved analyzing the stability of the enamide functionality by incubating model compound 9 (FIG. 5) in HDAC assay buffer at pH 2-12 for 60 min. The solutions were analyzed by HPLC at various time points during the incubation to determine the amount of conversion to the corresponding aldehyde (FIG. 5). At pH 4-12, there was no detectable conversion to the aldehyde after 60 min, indicating that the enamide is stable under physiological conditions. However, at pH 2, full conversion to the aldehyde was seen, verifying conversion of the enamide to the aldehyde following deacetylation, and highlighting the potential for use of enamides as an aldehyde protecting group in chemical synthesis.

Next, it was determined whether deacetylation, the first step of activity-based HDAC detection by HP-1, could be effected by recombinant HDAC enzymes. Incubation of HP-1 with HDAC isoforms was performed with or without the potent HDAC inhibitor suberoylanilide hydroxamic acid (SAHA) to verify that any detected deacetylation was a result of enzymatic activity.^{33,34} LCMS analysis indicated good conversion of HP-1 to DHP-1 in the presence of both HDAC1 and 3 isoforms with a k_{obs} with HDAC3 of 3.2×10⁻⁵±6×10⁻⁶ s⁻¹ and conversion t_{1/2} of ˜6 h. (Table 1, FIG. 6). By comparison, recombinant HDAC2, 6, and 8 as well as sirtuins 1 and 3 (HDAC Class III) did not deacetylate HP-1, giving HP-1 a distinct selectivity profile compared to activity-based probes designed around SAHA, a general Class I/II HDAC inhibitor.^{31,32} The enamide was assessed in more biologically relevant contexts. Before proceeding, the ‘off-target’ selectivity was assessed. To further test the selectivity of HP-1 deacetylation, HP-1 was incubated with enzymes from three different protease classes (serine, cysteine, and aspartate). These proteases, which were confirmed as being active with positive control substrates, were also unable to convert HP-1 to DHP-1, further indicating selectivity of HP-1 for a subset of Class I HDAC enzymes (Table 1). Analyses of LCMS traces from HP-1 deacetylation indicate that the cis-isomer of HP-1 is not deacetylated by HDAC1 or 3, suggesting that cis-HP-1 does not bind to these isoforms or that the isoforms are unable to deacetylate cis-HP-1 (FIG. 6). Deacetylation of HP-2 was also afforded by HDAC 1 and 3 but not by HDAC2, 6, or 8 (Table 2), indicating that the presence of the double bond in trans-HP-1 does not significantly alter the HDAC isoform selectivity.

TABLE 1
Unmasked aldehyde DHP-1 is produced by enzymatic deacetylation
Enzyme-catalyzed
aldehyde unmasking

% HP-1 to DHP-1

| Enzyme | −SAHA | +SAHA |
|---|---|---|
| HDAC1 | 15 | 0 |
| HDAC2 | 0 | 0 |
| HDAC3 | 93 | 0 |
| HDAC6 | 0 | 0 |
| HDAC8 | 0 | 0 |
| Sirtuin 1 | 0 | N/A |
| Sirtuin 3 | 0 | N/A |
| Chymotrypsin | 0 | N/A |
| Pepsin | 0 | N/A |
| Cathepsin B | 0 | N/A |

*Percentages indicate maximum detected conversion to DHP-1

In Table 1, percent conversion of HP-1 to DHP-1 by HDAC and Sirtuin enzymes and various proteases. A mixture of trans and cis isomers (1.5:1) of HP-1 was used for all assays; HDAC enzymes cleave only the trans isomer.

TABLE 2
Percent cleavage of HP-2 in vitro by
HDAC enzymes as determined by LCMS

Maximum % HP-2 to DHP-2

| Enzyme | −SAHA | +SAHA |
|---|---|---|
| HDAC1 | 6 | 0 |
| HDAC2* | 0 | 0 |
| HDAC3 | 87 | 0 |
| HDAC6 | 0 | 0 |
| HDAC8 | 0 | 0 |

*HP-2 and DHP-2 were not detected

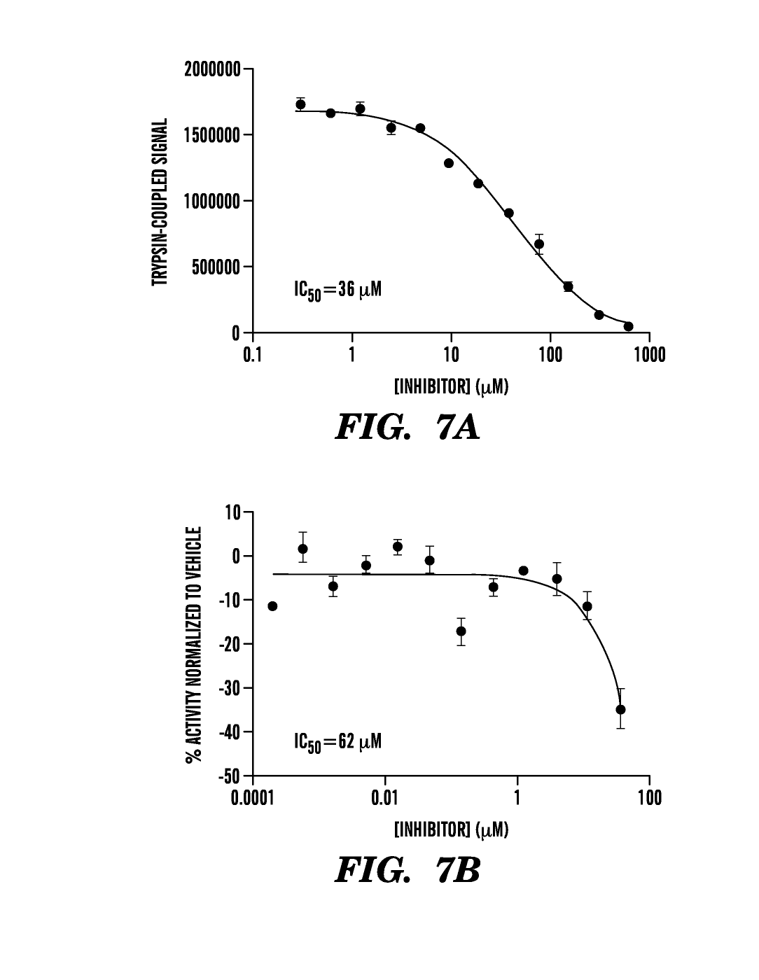

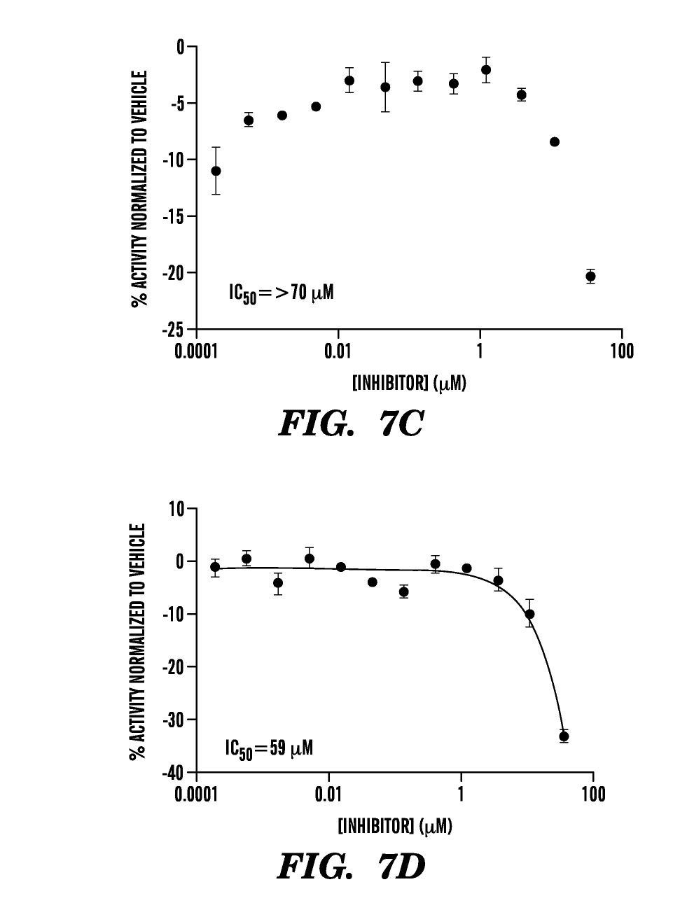

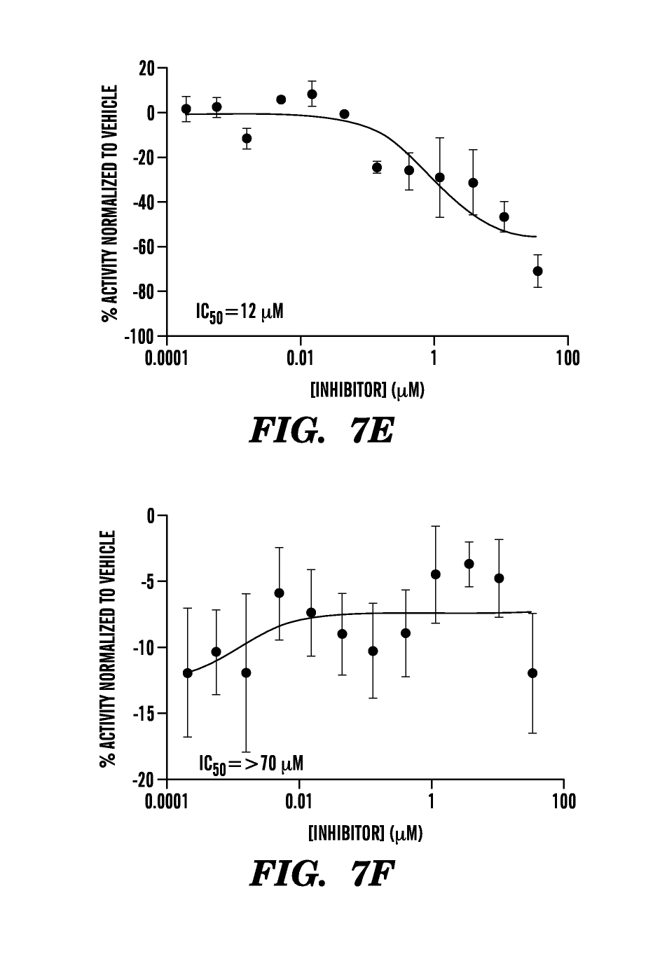

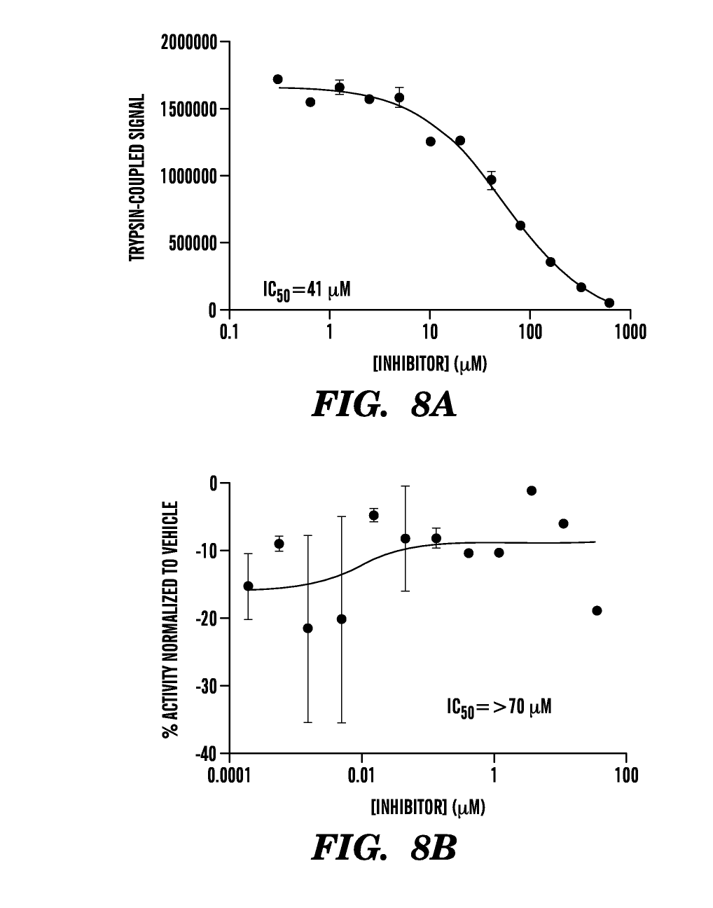

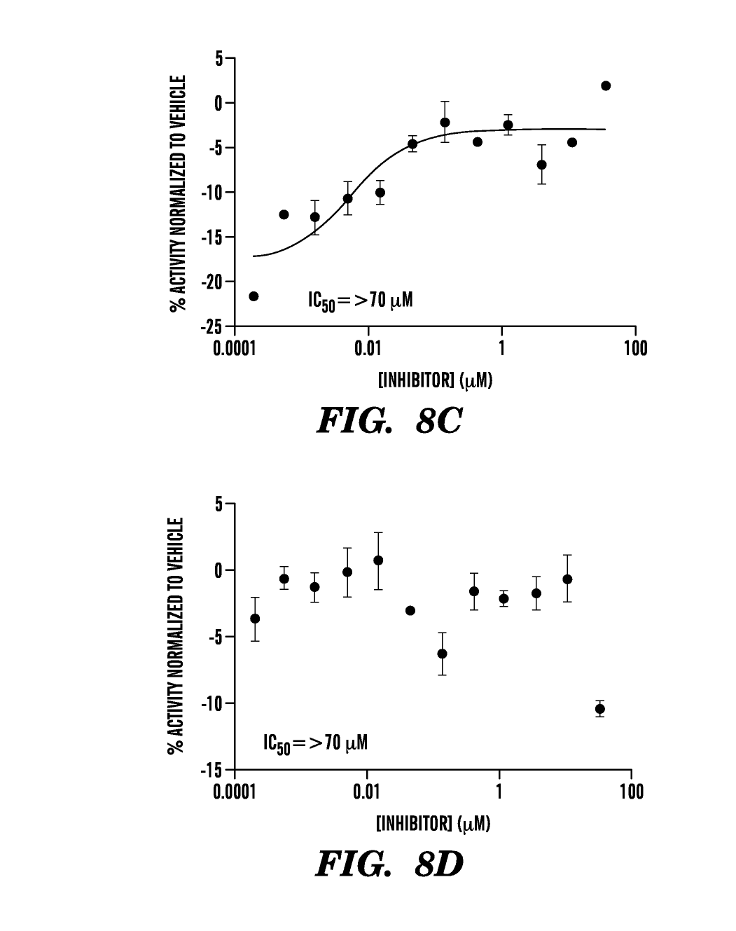

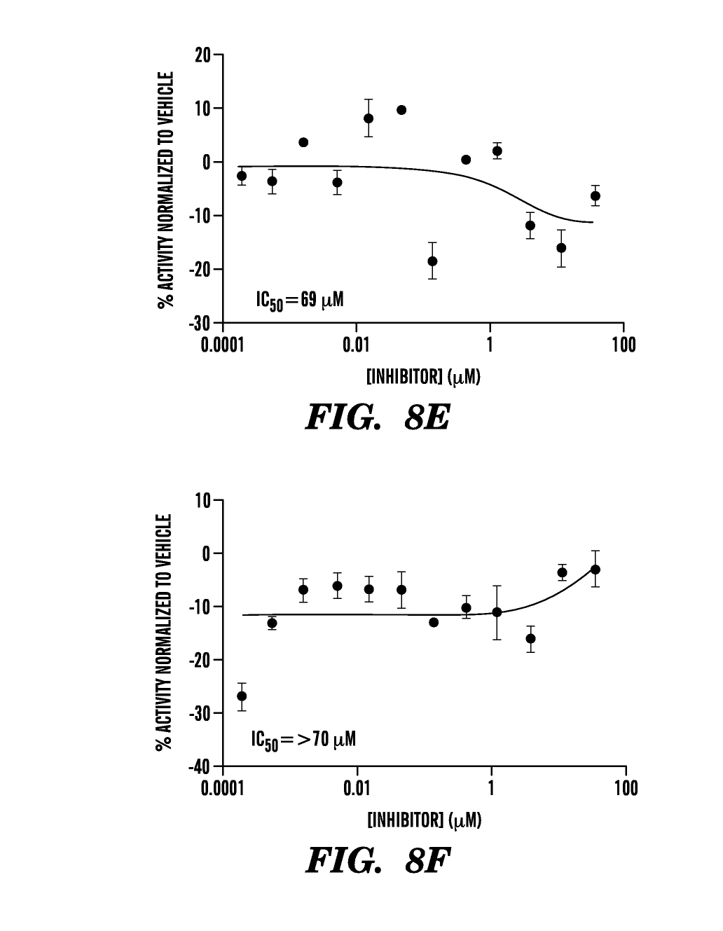

Competitive inhibition of HP-1 and 2 with a peptide substrate for HDAC isoforms was also examined to explore HDAC isoform selectivity. The measured IC₅₀ values indicate that some HDAC isoform selectivity may be related to binding affinity, as both HP-1 and 2 are deacetylated by and weakly inhibit HDAC1 (Table 3, FIGS. 7 and 8). However, both HP-1 and 2 are deacetylated by HDAC3, but only HP-1 has a detectable IC₅₀ for HDAC3. Furthermore, both HP-1 and 2 bind HDAC6, but neither were deacetylated by this isoform. Taken together, these data indicate that the selectivity of deacetylation of HP-1 and 2 is not dependent on binding affinity alone.

TABLE 3
HP-1 binds to three HDAC isoforms
HDAC Isoform
IC₅₀ Values

| Isoform | IC₅₀(μM) |
|---|---|
| HDAC1 | 35.8 |
| HDAC2 | >70 |
| HDAC3 | 59 |
| HDAC6 | 12 |
| HDAC8 | >70 |

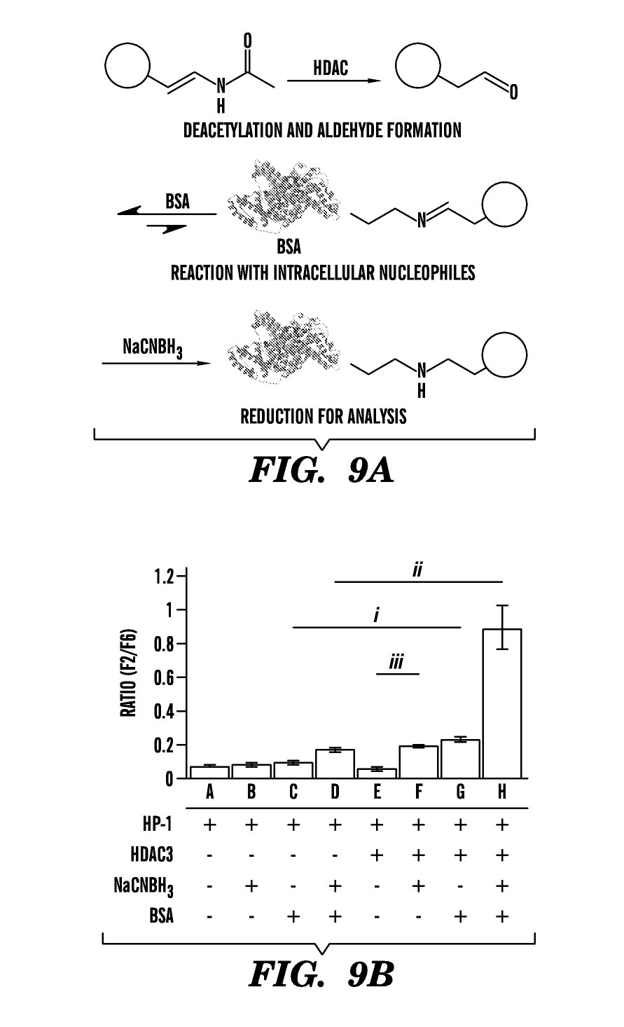

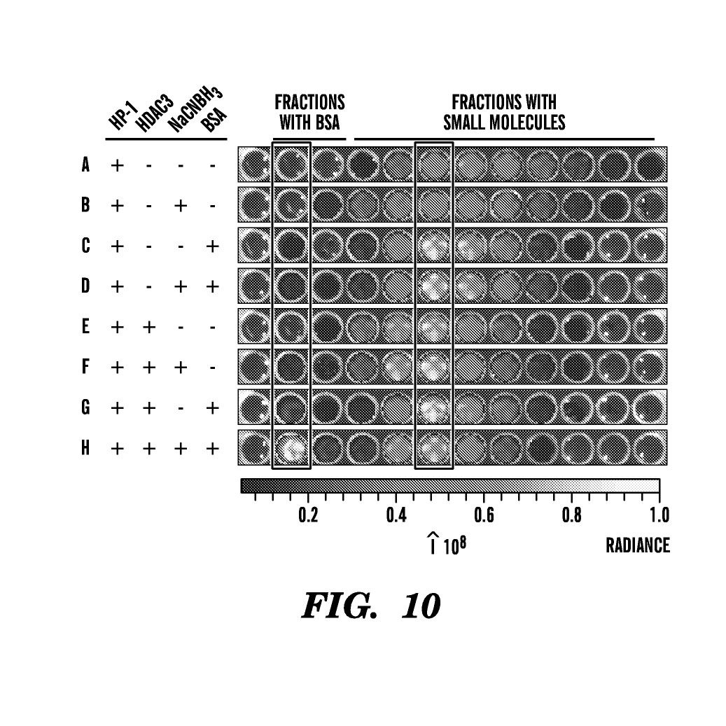

Following confirmation of HDAC-selective deacetylation of HP-1, it was verified that deacetylated HP-1 could covalently interact with proteins (FIG. 9A). Initially, HP-1 was incubated with HDAC3 to form DHP-1. Bovine serum albumin was then added to induce formation of covalent protein-DHP-1 bonds (i.e. imines), which resulted in a 2-fold increase in detected protein-DHP-1 binding relative to controls (FIG. 9B, lanes E and G, i; FIG. 10). Conditions were also tested with sodium cyanoborohydride (NaCNBH₃) in order to accumulate the protein-DHP-1 conjugates via imine reduction (FIG. 9A). These conditions showed a greater level of protein-DHP-1 binding, with a 5-fold increase compared to controls (FIG. 9B, Lanes F and H ii; FIG. 10). This detection of the covalent interactions between DHP-1 and adventitious nucleophiles of proteins demonstrates the potential for the DHP-1 aldehyde functionality to retain the deacetylated probe within cells.

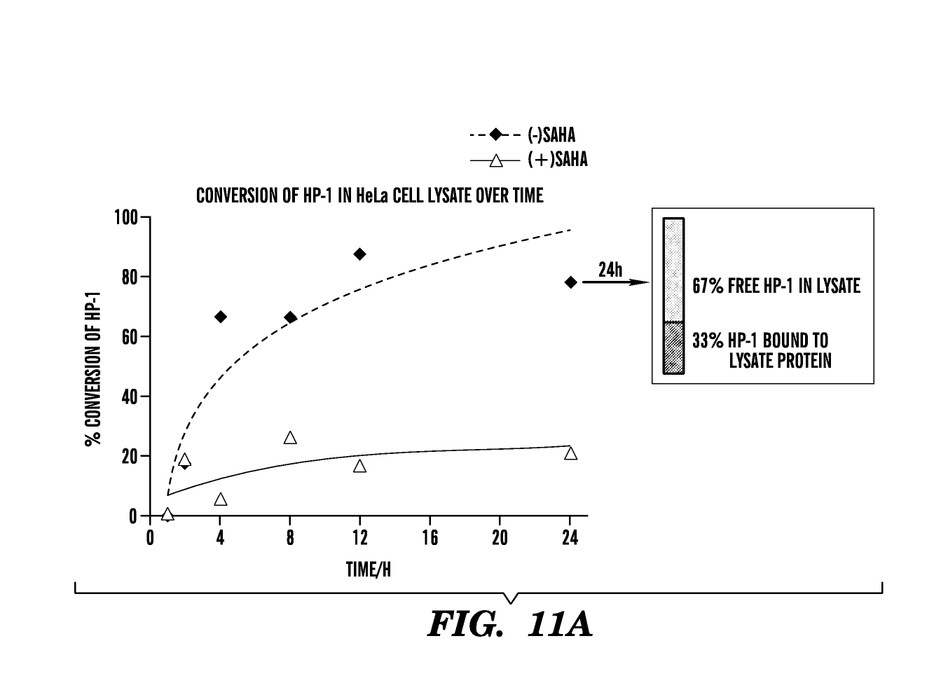

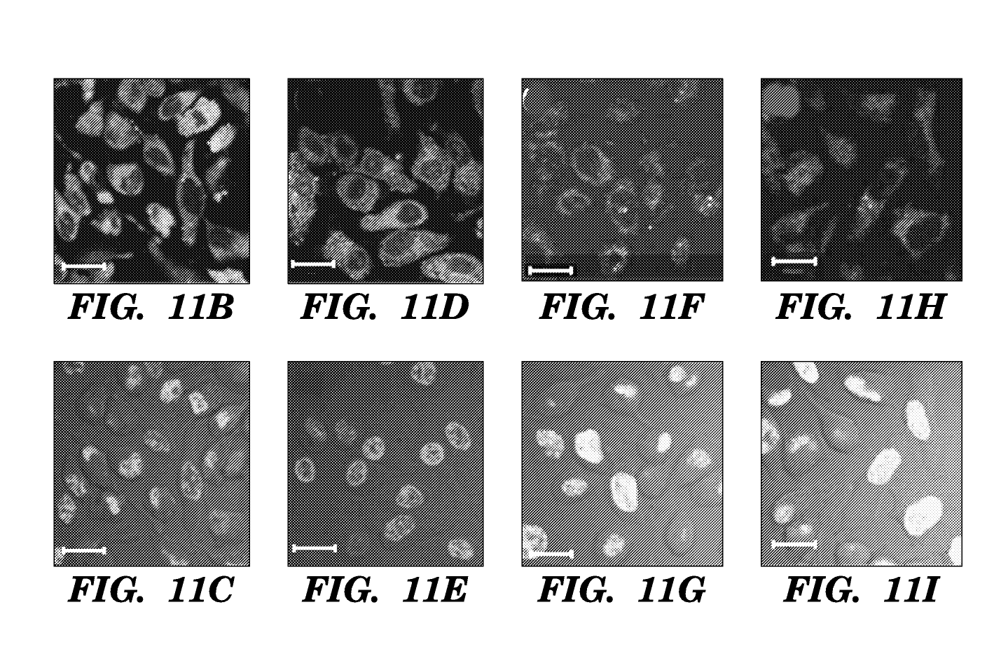

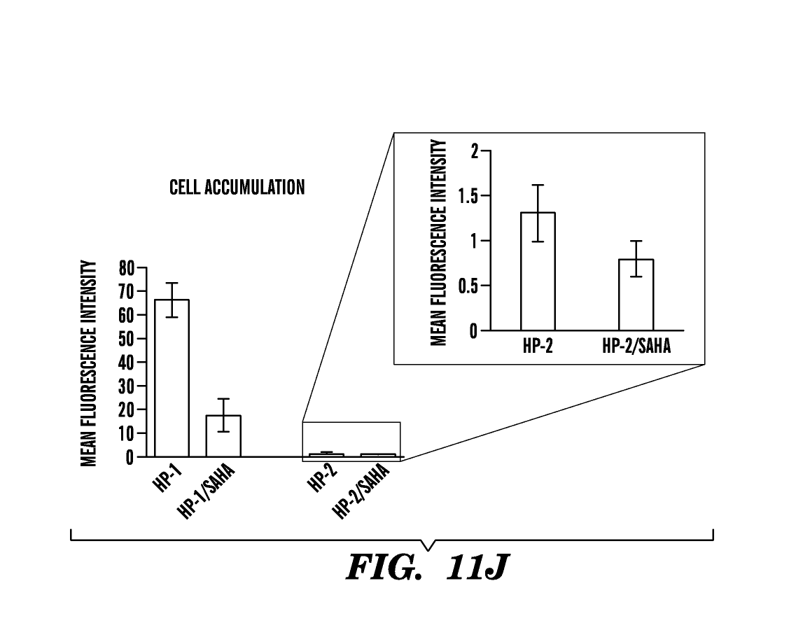

Initial examination of the HDAC-dependent deacetylation of HP-1 in a cellular context was carried out using HeLa whole-cell lysate and nuclear extract, as HeLa cells are known to have a high expression of HDACs.³⁵ HP-1 was converted to DHP-1 by both the whole-cell lysate and nuclear extract, and the production of DHP-1 was not detected following addition of the HDAC inhibitor SAHA (Table 4). Following this, the deacetylation of HP-1 and 2 was analyzed in live HeLa cells via incubation with the probes over 24 h in the absence or presence of SAHA. Analysis of cell supernatants and lysates by LCMS demonstrates that in the absence of SAHA 80% of HP-1 is cleaved over 24 h, forming a UV-active peak that is expected to be DHP-1 bound to one or several cellular nucleophiles. However, through separation of the lysate into protein-bound and unbound fractions it was determined that 33% of the intracellular fluorescent signal is attributable to the protein-bound probe (FIG. 11A). Additionally, the percent of HP-1 found in the lysate (2%) does not change after incubation of the cells with SAHA for 24 hours, indicating that HP-1 has reached and maintained an equilibrium between the intra- and extracellular space that is not altered by SAHA. Cleavage of HP-1 incubated with HeLa cells could be reduced to 20% over 24 h through addition of SAHA (FIG. 11B). By comparison, HP-2 incubated for 24 hours with HeLa cells has a 7.8% conversion to DHP-2 in the absence of SAHA and no detectable conversion to DHP-2 when SAHA is added.

TABLE 4
Hela cell nuclear and whole cell lysate-
induced HP-1 and HP-2 deacetylation

| | |
|---|---|
| HP-1 to DHP-1 | HP-2 to DHP-2 |

| Lysate | −SAHA | +SAHA | −SAHA | +SAHA |
|---|---|---|---|---|
| HeLa nuclear | + | − | + | − |
| extract | | | | |
| HeLa whole | + | − | + | − |
| cell | | | | |

To examine the activity-based cellular retention of HP-1 and 2, the probes were incubated with HeLa cells for 2 hours prior to confocal fluorescence imaging. Incubation was performed in the absence or presence of SAHA to probe the specificity of HP-1 retention for HDAC activity. As anticipated, HP-1 incubation in HeLa cells resulted in a robust intracellular fluorescent signal, while addition of SAHA reduces the level of fluorescence (FIGS. 11B-E, 11J), indicating that HP-1 deacetylation and cellular accumulation is sensitive to changes in HDAC activity. Interestingly, the HP-1 signal was localized to the cytoplasm, suggesting that HP-1 deacetylation occurred outside the nucleus or that DHP-1 diffused out of the nucleus and accumulated in the cytoplasm via interaction with intracellular nucleophiles. Given that HDAC3 showed the most activity with HP-1 in isolated enzyme experiments (Table 1), it is worth noting that HDAC3 is known to shuttle between the nucleus and cytoplasm.^{36,37} When HP-2 is utilized for HeLa cell imaging, fluorescence was not detected within the HeLa cells either in the absence or presence of SAHA (FIGS. 11F-11J), indicating that the trappable aldehyde released by HP-1 is essential for intracellular accumulation of the fluorescent NBD moiety and the detection of alterations in HDAC activity.

Taken together, the data indicate that HP-1 is a HDAC-selective fluorescent probe that contains a chemical moiety that confers increased intracellular retention following deacetylation by HDAC enzymes. To improve the performance of the probe, one skilled in the art can increase the rate of deacetylation and improve selectivity for a single HDAC isoform, which may be accomplished through structural modifications. It will also be critical to reduce the level of non-specific accumulation, while increasing the overall uptake.

In summary, a novel probe is developed for detection of HDAC activity that utilizes a unique aldehyde-trapping strategy for the accumulation of deacetylated HP-1 within cells. This accumulation results in increased fluorescence in cells with greater HDAC activity, thus affording a probe suitable for detection of HDAC activity via an activity-based cellular retention mechanism. When extrapolated to cells within an organism, this enamide-unmasking accumulation approach offers a mechanism for increased accumulation of the unmasked aldehyde and its attached cargo in cells and tissues with increased HDAC activity. Importantly, the cargo of the unmasked aldehyde can be easily adapted to contain tracers for positron emission tomography or contrast agents for magnetic resonance imaging, thus making the described enamide-accumulation approach a potential strategy for locating increased HDAC activity in vivo. Further, the aldehyde accumulation strategy could be modified to detect activity from other enzymes provided substrate catalysis can drive the unmasking of an aldehyde functional group.

### Methods

General methods. All chemical reagents were of ACS grade purity or higher, and used as received without further purification. Reactions were performed using standard techniques, including inert atmosphere of nitrogen with standard Schlenk technique, when necessary. Glassware was oven-dried at 150° C. overnight. Analytical thin layer chromatography (TLC) was performed on SiliCycle TLC silica Gel 60-F254 plates with visualization by ultraviolet (UV) irradiation at 254 nm. Purifications were performed using HP silica chromatography column by Teledyne Isco. The elution system for each purification was determined by TLC analysis. Chromatography solvents were purchased from commercial sources and used without distillation. NMR spectra were recorded at 22° C. on a Varian 500 MHz spectrometer (¹H, 500.16 MHz and ¹³C, 125.784 MHz). ¹H and ¹³C NMR chemical shifts are reported as δ in units of parts per million (ppm) utilizing residual solvent signals for referencing. HPLC-analysis of organic synthetic reactions was conducted on an Agilent 1100 series HPLC and mass spectrometry data were recorded on an Agilent 6310 ion trap mass spectrometer (ESI source).

Synthesis of HP-1. HP-1 and 2 were synthesized in 7 and 2 synthetic steps respectively. Detailed syntheses of HP-1 and 2 are reported in Example 1.

LCMS characterization of HDAC enzymatic action on HP-1 and HP-2. The enzymatic cleavage of HP-1 and HP-2 was analyzed by LCMS assays with HeLa nuclear extract (AnaSpec), HeLa whole cell lysate (Santa Cruz Inc.), and the purified HDAC isoforms: 1, 2, 3, 6 and 8 (HDAC1, 3 and 8 from Cayman Chemicals and HDAC2 and 6 provided by Dr. Stephen Haggarty). Each sample in HDAC buffer was incubated for 12 hours at 37° C. Following incubation, an aliquot of supernatant from each sample was analyzed by LCMS. Deacetylation of HP-1 and 2 was confirmed by detection of the (M+H)⁺ ion following positive electrospray ionization. The peak area for each detected compound was measured to determine the % deacetylation of each probe (full experimental details in Example 1).

LCMS analysis of HP-1 cleavage by HDAC3 enzyme over time and determination of the observed rate constant (k_{obs}). The rate of cleavage of HP-1 by purified HDAC3 was analyzed by performing a LCMS assay. Solutions containing HP-1 and HDAC3 in HDAC buffer were incubated for 12 hours at 37° C. and aliquots of supernatant from each sample was analyzed by LCMS at t=0, 1, 2, 4, 8, and 12 h. Deacetylation of HP-1 was confirmed by detection of the (M+H)⁺ ion following positive electrospray ionization. The peak area for each detected compound was measured to determine the % deacetylation. The observed rate constant was determined using Graphpad by plotting Ln(DHP-1 peak area) versus time (full experimental details in Example 1).

Characterization of sirtuin (HDAC Class III) and protease enzymatic action on HP-1. The enzymatic cleavage of HP-1 was analyzed by performing LCMS assays with sirtuin 1 and 3 (Cayman Chemicals) and the proteases chymotrypsin (Sigma-Aldrich), cathepsin (EMD Millipore), and pepsin (Sigma-Aldrich) (full experimental details in Example 1).

IC₅₀ measurements. HP-1 and HP-2 IC₅₀ values for HDAC1 were determined using the Trypsin-coupled assay as well as the Caliper endpoint assay. HP-1 and HP-2 IC₅₀ values for HDAC2, HDAC3, HDAC6 and HDAC8 were determined with the Caliper endpoint assay (full experimental details in Example 1).

HP-1 deacetylation and protein binding assay. Solutions containing HP-1 (20 μM) in 30 μL HDAC buffer with 5% DMSO were incubated at 37° C. for four hours in the presence or absence of HDAC3 (3.6 μM). After incubation with HDAC3, NaCNBH₃ (1.4 mM) or vehicle (H₂O) and BSA (6 mg/mL) or vehicle (HDAC buffer), were added to the solutions prior to an additional incubation at 37° C. for two hours. Following the second incubation, the final samples were separated by G-25 columns (GE Healthcare, Buckinghamshire, UK) and the eluent was collected separately to obtain fractions 2-12. The fractions were transferred to a well in a 96-well, black, clear-bottom plate (Corning Incorporated, Corning, N.Y.) and the fluorescence was detected using an IVIS Spectrum (Caliper, Hopkinton, Mass.). To obtain the fluorescence signals, the 465 nm excitation filter, 530 nm emission filter, and a 5 second exposure were used. For analysis, the total photon flux over the area of each well was determined (full experimental details in Example 1).

HDAC Activity in HeLa Cells with HP-1

HeLa cell culture and treatment with HP-1 and HP-2. HeLa cells (ATCC) were grown as a monolayer in Eagles Minimum Essential Medium (EMEM, GIBCO, BRL, Gaithersburg, Md.) with 10% Fetal Bovine Serum (FBS, GIBCO, BRL, Gaithersburg, Md.) and 1% penicillin/streptomycin (100 mg/mL). All cell culture dishes were maintained in a humidified atmosphere with 5% CO₂ at 37° C.

Determination of HDAC activity in HeLa cells by LC-MS. HeLa cells grown in 600 mL cell culture flasks were treated with HP-1 or 2±SAHA so that the final concentrations were 5 μM for HP-1 and 2 and 10 μM for SAHA. Incubations were in HDAC buffer with 0.01% DMSO) at 37° C. for t=1, 2, 4, 8, and 12 h and 24 h. Following incubation, the medium was removed and the cells were washed three times with DPBS buffer. Cells were scraped off of the flask, lysed in Millipore water using a mechanical homogenizer, and supernatant of the lysed samples were analyzed by LC-MS. Cleavage and accumulation was confirmed by detection of the (M+H)⁺ ion following positive electrospray ionization. The peak area for each detected compound was measured to determine the % conversion versus time (full experimental details in Example 1).

Determination of HDAC activity in HeLa cells by fluorescence. HeLa cells grown in 600 mL cell culture flasks were treated with HP-1 or 2±SAHA so that the final concentrations were 5 μM for HP-1 and 2 and 10 μM for SAHA. Incubations were in HDAC buffer with 0.01% DMSO) at 37° C. for t=1, 2, 4, 8, and 12 h and 24 h. Following incubation, the medium was removed and the cells were washed three times with DPBS buffer. Cells were scraped off of the flask and lysed in Millipore water using a mechanical homogenizer and protein bound probe fraction of cell lysate was separated by Micron centrifugal filter device. Each cell lysate sample (before and after separation) was transferred to a well in a 96-well, black, clear-bottom plate (Corning Incorporated, Corning, N.Y.) and the fluorescence was detected using an IVIS Spectrum (Caliper, Hopkinton, Mass.). To obtain the fluorescence signals, the 465 nm excitation filter, 530 nm emission filter, and a 1 second exposure were used. For analysis, the total photon flux over the area of each well was determined (full experimental details in Example 1).

Imaging HDAC activity in HeLa cells with HP-1 and 2. An acid-washed, poly-lysine-treated sterile glass cover slip was added to each well of 6-well plate, and HeLa cells were plated at a seeding density of ˜2.5×10⁵ cells/mL in 2 mL of growth medium. After 24 hours, the cells reached 80-85% confluence. Cells were treated with HP-1, HP-1 with SAHA, HP-2, or HP-2 with SAHA so that the final concentrations of HP-1 and HP-2 were 5 μM in HDAC buffer (with 0.01% DMSO) and incubated at 37° C. for 2 hours. Following incubation, the medium was removed, the cells were washed three times with HDAC buffer, and fixed with 4% paraformaldehyde in PBS. Cover slips were mounted on a drop of Gel Mount (anti-fade with DAPI nuclear stain) and analyzed by confocal imaging.

Confocal fluorescence imaging. Confocal fluorescence imaging was performed with a Zeiss laser scanning microscope 710 with a 63× objective lens and Zen 2009 software (Carl Zeiss). HP-1 and HP-2 were excited using a 488 nm Ar laser, and emission was collected using a META detector between 500 and 650 nm (full experimental details in Example 1).

### References for Example 2:

1. Johnsson, N.; Johnsson, K., (2007) Chemical tools for biomolecular imaging. ACS Che.Biol. 2 (1), 31-38.

2. Prescher, J. A.; Bertozzi, C. R.,(2005) Chemistry in living systems. Nat.Chem.Biol. 1 (1), 13-21.

3. Kobayashi, H.; Ogawa, M.; Alford, R.; Choyke, P. L.; Urano, Y., (2010) New strategies for fluorescent probe design in medical diagnostic imaging. Chem. Rev. 110 (5), 2620-2640.

4. Baruch, A.; Jeffery, D. A.; Bogyo, M., (2004) Enzyme activity—it's all about image. Trends Cell Bio1.14 (1), 29-35.

5. Blum, G.; Mullins, S. R.; Keren, K.; Fonovi\check{c}, M.; Jedeszko, C.; Rice, M. J.; Sloane, B. F.; Bogyo, M., (2005) Dynamic imaging of protease activity with fluorescently quenched activity-based probes. Nat.Chem.Biol. 1 (4), 203-209.

6. Weissleder, R.; Tung, C.-H.; Mahmood, U.; Bogdanov, A., (1999) In vivo imaging of tumors with protease-activated near-infrared fluorescent probes. Nat. Biotech. 17 (4), 375-378.

7. Wysocki, L. M.; Lavis, L. D., (2011) Advances in the chemistry of small molecule fluorescent probes. Curr. Opin. Chem. Biol. 15 (6), 752-759.

8. Tian, L.; Yang, Y.; Wysocki, L. M.; Arnold, A. C.; Hu, A.; Ravichandran, B.; Sternson, S. M.; Looger, L. L.; Lavis, L. D., (2012) Selective esterase-ester pair for targeting small molecules with cellular specificity. P. Natl. Acad. Sci. USA. 109 (13), 4756-4761.

9. Yeh, H.-H.; Tian, M.; Hinz, R.; Young, D.; Shavrin, A.; Mukhapadhyay, U.; Flores, L. G.; Balatoni, J.; Soghomonyan, S.; Jeong, H. J., (2012) Imaging epigenetic regulation by histone deacetylases in the brain using PET/MRI with 18 F-FAHA. NeuroImage 64, 630-639.

10. Cheng, T.-C.; Roffler, S. R.; Tzou, S.-C.; Chuang, K.-H.; Su, Y.-C.; Chuang, C.-H.; Kao, C.-H.; Chen, C.-S.; Harn, I.-H.; Liu, K.-Y., (2012) An activity-based near-infrared glucuronide trapping probe for imaging β-glucuronidase expression in deep tissues. J. Am.Chem. Soc. 134 (6), 3103-3110.

11. Baba, R.; Hori, Y.; Mizukami, S.; Kikuchi, K., (2012) Development of a Fluorogenic Probe with a Transesterification Switch for Detection of Histone Deacetylase Activity. J. Am.Chem. Soc. 134, 14310-14313.

12. Sasaki, K.; Ito, A.; Yoshida, M., (2012) Development of live-cell imaging probes for monitoring histone modifications. Bioorga.Med. Chem. 20 (6), 1887-1892.

13. Coleman, R. E., (2000) FDG imaging. Nucl.Med.Biol. 27 (7), 689-690.

14. Ido, T.; Wan, C.; Fowler, J.; Wolf, A., (1977) Fluorination with molecular fluorine. A convenient synthesis of 2-deoxy-2-fluoro-D-glucose. The Journal of Organic Chemistry 42 (13), 2341-2342.

15. Hooker, J. M., (2010) Modular strategies for PET imaging agents. Curr. Opin. Chem.Biol. 14 (1), 105-111.

16. Roth, S. Y.; Denu, J. M.; Allis, C. D., (2001) Histone acetyltransferases. Annu Rev.Biochem. 70 (1), 81-120.

17. Allfrey, V.; Faulkner, R.; Mirsky, A., (1964) Acetylation and methylation of histones and their possible role in the regulation of RNA synthesis. P. Natl. Acad. Sci. USA. 51 (5), 786.

18. Balch, C.; Fang, F.; Matei, D. E.; Huang, T. H.-M.; Nephew, K. P., (2009) Minireview: epigenetic changes in ovarian cancer. Endocrinology 150 (9), 4003-4011.

19. Strahl, B. D.; Allis, C. D., (2000) The language of covalent histone modifications. Nature 403 (6765), 41-45.

20. Agalioti, T.; Chen, G.; Thanos, D., (2002) Deciphering the transcriptional histone acetylation code for a human gene. Cell 111 (3), 381-392.

21. Ellis, L.; Atadja, P. W.; Johnstone, R. W., (2009) Epigenetics in cancer: targeting chromatin modifications. Mol. Can. Ther. 8 (6), 1409-1420.

22. Hendrich, B.; Bickmore, W., (2001) Human diseases with underlying defects in chromatin structure and modification. Hum.Mol.Genet. 10 (20), 2233-2242.

23. Kuo, M.-H.; Allis, C. D., (1998) Roles of histone acetyltransferases and deacetylases in gene regulation. Bioessays 20 (8), 615-626.

24. Minucci, S.; Pelicci, P. G., (2006) Histone deacetylase inhibitors and the promise of epigenetic (and more) treatments for cancer. Nat. Rev. Cancer 6 (1), 38-51.

25. Rodenhiser, D.; Mann, M., (2006) Epigenetics and human disease: translating basic biology into clinical applications. Can. Med.Assoc. J. 174 (3), 341-348.

26. Grayson, D. R.; Kundakovic, M.; Sharma, R. P., (2010) Is there a future for histone deacetylase inhibitors in the pharmacotherapy of psychiatric disorders? Mol.Ppharmacol. 77 (2), 126-135.

27. Gray, S. G., (2011) Epigenetic treatment of neurological disease. Epigenomics 3 (4), 431-450.

28. Butler, R.; Bates, G. P., (2006) Histone deacetylase inhibitors as therapeutics for polyglutamine disorders. Nat. Rev. Neurosci. 7 (10), 784-796.

29. Dietz, K. C.; Casaccia, P., (2010) HDAC inhibitors and neurodegeneration: at the edge between protection and damage. Pharmacol. Res. 62 (1), 11-17.

30. Sadri-Vakili, G.; Cha, J.-H. J., (2006) Histone Deacetylase Inhibitors: A Novel Therapeutic Approach to Huntingtons Disease (Complex Mechanism of Neuronal Death). Curr. Alzheimer Res. 3 (4), 403-408.

31. Salisbury, C. M.; Cravatt, B. F., (2007) Activity-based probes for proteomic profiling of histone deacetylase complexes. P.Natl Acad. Sci. USA. 104 (4), 1171-1176.

32. Salisbury, C. M.; Cravatt, B. F., (2008) Optimization of activity-based probes for proteomic profiling of histone deacetylase complexes. J.Am. Chem. Soc. 130 (7), 2184-2194.

33. Grant, S.; Easley, C.; Kirkpatrick, P., Vorinostat. (2007) Nat. Rev. Drug Discov. 6 (1), 21-22.

34. Mann, B. S.; Johnson, J. R.; Cohen, M. H.; Justice, R.; Pazdur, R., (2007) FDA approval summary: vorinostat for treatment of advanced primary cutaneous T-cell lymphoma. Oncologist 12 (10), 1247-1252.

35. Dueilas-Gonzalez, A.; Lizano, M.; Candelaria, M.; Cetina, L.; Arce, C.; Cervera, E., (2005) Epigenetics of cervical cancer. An overview and therapeutic perspectives. Mol.Cancer 4 (1), 38.

36. Khochbin, S.; Verdel, A.; Lemercier, C.; Seigneurin-Berny, D., (2001)Functional significance of histone deacetylase diversity. Curr.Opin.Genet.Dev. 11 (2), 162-166.

37. Yang, W.-M.; Tsai, S.-C.; Wen, Y.-D.; Fejer, G.; Seto, E., (2002) Functional domains of histone deacetylase-3. J. Biol. Chem. 277 (11), 9447-9454.

1. A compound characterized in having a structure: Lab-L-Ena, wherein Lab is a detectable label, L is a linker, and Ena is an enamide group.

2. The compound of claim 1, corresponding to Formula I: 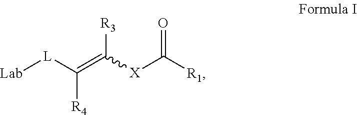
wherein:
X is O, S, or NR₂; and
R₁, R₂, R₃, and R₄ are each independently hydrogen, deuterium, halogen, hydroxyl, nitro, cyano, isocyano, thiocyano, isothiocyano, aryl, alkyl, perfluorinated alkyl, alkenyl, perfluorinated alkenyl, alkynyl, perfluorinated alkynyl, alkoxy, alkylthioxy, amino, monoalkylamino, dialkylamino, acyl, carbonyl, carboxyl, azide, sulfinyl, sulfonyl, sulfino, sulfo, or thiol, each of which can be optionally substituted and each of which can optionally comprise a stable isotope.

3. The compound of claim 1, wherein the detectable label is an imagining agent or a contrast agent.

4. The compound of claim 1 wherein the detectable label is selected from a group consisting of an optical reporter, non-metallic isotope, a paramagnetic metal ion, a ferromagnetic metal, echogenic substance (either liquid or gas), a boron neutron absorber, a gamma-emitting radioisotope, a positron-emitting radioisotope, an x-ray absorber, fluorescent molecules, radioisotopes, nucleotide chromophores, enzymes, enzyme substrates, chemiluminescent moieties, magnetic particles, bioluminescent moieties, nucleic acids, antibodies, and any combinations thereof.

5. (canceled)

6. The compound of claim 5, corresponding to Formula II: 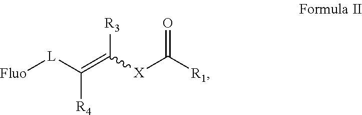
wherein:
X is O, S, or NR₂;
R₁, R₂, R₃, and R₄ are each independently hydrogen, deuterium, halogen, hydroxyl, nitro, cyano, isocyano, thiocyano, isothiocyano, aryl, alkyl, perfluorinated alkyl, alkenyl, perfluorinated alkenyl, alkynyl, perfluorinated alkynyl, alkoxy, alkylthioxy, amino, monoalkylamino, dialkylamino, acyl, carbonyl, carboxyl, azide, sulfinyl, sulfonyl, sulfino, sulfo, or thiol, each of which can be optionally substituted and each of which can optionally comprise a stable isotope; and
Fluo is a fluorescent molecule.

7. The compound of claim 5, wherein the fluorescent molecule comprises hydroxycoumarin, aminocoumarin, methoxycoumarin, cascade blue, pacific blue, pacific orange, lucifer yellow, nitrobenzoxadiazole (NBD), R-phycoerythrin, PE-Cy5 conjugates, PE-Cy7 conjugates, Red 613, PerCP, TruRed, FluorX, Fluorescein, BODIPY, Cy2, Cy3, Cy3B, Cy3.5, Cy5, Cy5.5, Cy7, SeTau-647, TRITC, rhodamine, Texas Red, allophycocyanin (APC), APC-Cy7 conjugates, or derivatives thereof.

8. The compound of claim 7, wherein the fluorescent molecule comprises NBD, and wherein the compound corresponds to Formula III: 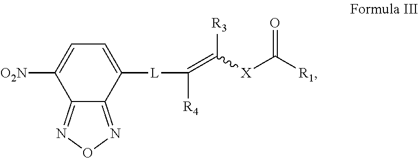
wherein:
X is O, S, or NR₂; and
R₁, R₂, R₃, and R₄ are each independently hydrogen, deuterium, halogen, hydroxyl, nitro, cyano, isocyano, thiocyano, isothiocyano, aryl, alkyl, perfluorinated alkyl, alkenyl, perfluorinated alkenyl, alkynyl, perfluorinated alkynyl, alkoxy, alkylthioxy, amino, monoalkylamino, dialkylamino, acyl, carbonyl, carboxyl, azide, sulfinyl, sulfonyl, sulfino, sulfo, or thiol, each of which can be optionally substituted and each of which can optionally comprise a stable isotope.

9. The compound of claim 1, wherein the linker is selected from the group consisting of: —O—, —S—, —S—S—, —C(O)—, —C(O)O—, —C(O)NR^{a}—, —SO—, —SO₂—, —SO₂NR^{a}—, substituted or unsubstituted alkyl, substituted or unsubstituted alkenyl, substituted or unsubstituted alkynyl, arylalkyl, arylalkenyl, arylalkynyl, heteroarylalkyl, heteroarylalkenyl, heteroarylalkynyl, heterocyclylalkyl, heterocyclylalkenyl, heterocyclylalkynyl, aryl, heteroaryl, heterocyclyl, cycloalkyl, cycloalkenyl, alkylarylalkyl, alkylarylalkenyl, alkylarylalkynyl, alkenylarylalkyl, alkenylarylalkenyl, alkenylarylalkynyl, alkynylarylalkyl, alkynylarylalkenyl, alkynylarylalkynyl, alkylheteroarylalkyl, alkylheteroarylalkenyl, alkylheteroarylalkynyl, alkenylheteroarylalkyl, alkenylheteroarylalkenyl, alkenylheteroarylalkynyl, alkynylheteroarylalkyl, alkynylheteroarylalkenyl, alkynylheteroarylalkynyl, alkylheterocyclylalkyl, alkylheterocyclylalkenyl, alkylhererocyclylalkynyl, alkenylheterocyclylalkyl, alkenylheterocyclylalkenyl, alkenylheterocyclylalkynyl, alkynylheterocyclylalkyl, alkynylheterocyclylalkenyl, alkynylheterocyclylalkynyl, alkylaryl, alkenylaryl, alkynylaryl, alkylheteroaryl, alkenylheteroaryl, alkynylhereroaryl; wherein backbone of the linker can be interrupted or terminated by O, S, S(O), SO₂, N(R^{a})₂, C(O), C(O)O, C(O)NR^{a}, cleavable linking group, substituted or unsubstituted aryl, substituted or unsubstituted heteroaryl, substituted or unsubstituted heterocyclic, and wherein R^{a} is hydrogen, acyl, aliphatic or substituted aliphatic.

10. The compound of claim 8, corresponding to Formula IV:

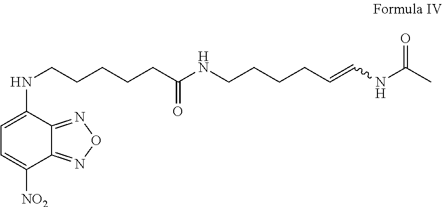 [SMILES: CC(=O)NC=CCCCCNC(=O)CCCCCNc1ccc([N+](=O)[O-])c2nonc12]

11. The compound of claim 1, wherein the compound is a trans-isomer.

12. A method of detecting enzyme activity of a deacetylase enzyme, the method comprising
(i) contacting the deacetylase enzyme with a compound of claim 1; and
(ii) determining the deacetylase activity by measuring a signal produced by a fragment of the compound.

13. The method of claim 12, wherein the deacetylase enzyme is a histone deacetylase (HDAC) or a sirtuin.

14. The method of claim 13, wherein the deacetylase enzyme is one of Class I HDAC enzymes.

15. The method of claim 14, wherein the deacetylase enzyme is HDAC1, HDAC3, or a combination thereof.

16. (canceled)

17. The method of claim 12, wherein the fragment of the compound is produced by the deacetylase enzyme cleaving the compound.

18. The method of claim 12, wherein the contacting is ex vivo or in vivo.

19. (canceled)

20. (canceled)

21. (canceled)

22. (canceled)

23. (canceled)

24. (canceled)

25. A method of screening a substance for its effect on deacetylase enzyme activity, the method comprising:
contacting the substance with a deacetylase enzyme;
(ii) contacting the deacetylase enzyme with a compound of claim 1; and
(iii) determining the effect of the substance on deacetylase enzyme activity by measuring and comparing a signal produced by a fragment of the compound relative to a control, wherein the control is performed in the absence of the substance.

26. (canceled)

27. (canceled)

28. (canceled)

29. (canceled)

30. (canceled)

31. (canceled)

32. (canceled)

33. (canceled)

34. (canceled)

35. A method of targeting a cell comprising a deacetylase enzyme within a cell population, the method comprising contacting the cell population with a compound of claim 1.

36. A method of delivering a drug to a cell comprising a deacetylase enzyme, the method comprising contacting the cell with a composition comprising the drug linked to an enamide group.

37. (canceled)

38. (canceled)

39. (canceled)

40. (canceled)
